SEQUENCE LISTING (79 sequences)

SEQ ID NO: 1 - DNA, 1840 bp, Homo sapiens
  misc_feature 1..1840: note = lncFIB #11: ENSG00000249406
  source 1..1840: mol_type = other DNA
atagagagttggtgactgcctttgagaggagaatggttgtgatccttttgccgagaactcacccagaggacttataccttggtgacagcagaaggactcaagttagaaggccagggcccttaaagggaggggacgattcgaggaaagtatagattagaaaactgaggcccagagaaggaaagtagctgccagaggtcacacagctaggattgagggatcagaggttgcacccttctaggaacttccgcctgagggagactggtcagagctcaggagggactgttccaggggatgagatatctgctggccaggaaaggcatgggaactgggcagcggagcacaccctccggacctggcccaacttcagaataacaacaaatgattgtgccaggctctgccagttcctcctcccccacaagctggcacatgtggatattggctctgttttgcctttgacccctagtgaaagaaaatactgagacggagtctcgctctgtcgcccaggttggagtgaagcggctagatctcagctcactgcaacctctgcctcccaggtttaagcaattctcctgtctcagcctccagagtagctgggactacaggcgcatgccaccatgccctactaattttttgtatttttaggagagacaggccttcaccatgttggtcaagctggtctcgaactcctgacctcaggtgacccaccggcttcagcctcccaaagtgctgggattacaggcatgagccaccgcgcccagcctcatatctatctttatcactccatttcagatcttcttccatatatggctctgtgtgtctcctgtccttccctgtctcctcactgcttttcagctggtagacccctgtggggacaacagatcagagcttggtcaccaatatggacatctaggaccacaggtcatcctcaagccttcctcccaggctatccttgtccctgaatactaatgtctaaaggtcagcccagctaccagcagtgtgcatgtgatgtgtcccatctccccttcggattggaggaagaagctgtgtccctcccatcagacagacggtctccttaaagattctcctggtctccagcttgggctctgctggaaagaagaggaagtatttagcaagggtgaagatgggttcttacttccctgaggcaaccagagataagaaaagaccagaatatgaatatccagcattccacaaagaatcctacaggcagggatattgcccaatcaccagtgcctcgcatcccagttctatccaagatccaagcttgatatcctcctgaaagacttcctgaatcaggtcaccaacatggacatctgaacttccagaatcaggtcaccgtctttcttctcatctctggatgtatttggagctcatttgtctgacggtttttgaatgctatcttttaaaaataaacttttacaatttgggtttaataattctagatttatagaaaagttgaaaagaaagtacagaaagttcccatatatcccttatccagttctcttaatgttaacctcctacatcatcatggaacatttgccaaaatgaagatgtcaacactggtgctttgctgtgaactaaactccagactttattcggatttcatgaatttttctcctaaggtcccttttccgttccggcattccacccaggatatcacattgcatttggtgtttgtgtctccttggtctcctctggttcttgagagtttctcagtctctccttgtttttcgtgactgaagtttcaaggagtactggtcaggtattccatagaatgtcctactgttagctttcagcagtaaaagcctttccacaaacaaaa

SEQ ID NO: 2 - DNA, 7060 bp, Homo sapiens
  misc_feature 1..7060: note = lncFIB #10: ENSG00000260604
  source 1..7060: mol_type = other DNA
agaggagtgtgcggaaagatggagttgagacagaaacttgttagcataacaccgaaggagttttgctgtgagtagcatcagcatagcaggagaacatccctagctctcctgccgctgctctgccacaaggagagaaagggaaaagcaaattcagcaggacctctaggactttgctgtagatctccaggtggagccaggaccctccaggagaactgatggctagaaaagactgcaagtaatgtggaggaaggaagaagagagagagcagagagagatgattcttgcaaacattgatctgtgtagagcggatgtcaacttgatgggataagacctgggttgacttggtatctgggttcaggtcagcaaactctaacggcatttccttctactagcagacagatctgctcatcctgcgacattgtggacagtgctgaatggcttccagaacattcctcttatgtctactggttaaactttgaagcatctgaaccatttgggagagcagggacccccatgaacaggagatactggaggtgtctgaggccacttcatgccctttctcaagagtggcctgcaggtctttcagccactggccactgaagtccagctcctgccagccatggatttgggcactgggctccagtgttgagaaagttcttgacttccagcgcttgttttcctagtggacaataggagtcagagacagagagggagagtgaagatgcagaagacatatcctttccattgtgacccagataagaatgtagccggtgagcttgcttcctgtcactcatttcccagctatttgttgtagtccaagttgtgcatattctttctgttaggttcttatgtttgtcttaagctcacccatcttttcttcttccctcattttttctttataaatcaagaaaaattctgccactttctttccctggctcactgcacagccaatcaataattttgaatcctttgtgtatcaatttctactggctattcattatttgtggaaaccatattaacgggtccataaaaatcacagcaatccatttgttaaattcatcataagaacagattacatctttctggccaagtttcatggtttggtaatatcatattttggttttagaatttataaggtgcattctcccatacttttcaagtatattttttattcattgccctctctgaattaggcatttttggggatgcacgttttacagatatggagacaatggttcagagaaatcagcttgttcacagtggggccagagctgggacctagggcttctgactctatgtctcaggtcattcctatcacacacacactgaaggagaaaagagttcccttgtctggagaagacatatcaaattcatgcgggttaaacacagaaattccaaatagctgtagtttttgtaaaaatggttgagctatggacttttaaagtagttggccacctgtggtggtgggtggaagggagttagggcaggattgttgtgcttcagggaggattcagtgtctgcacgaccacggaggggggtgcagggcaccaggtatccaggactgttgtcaagagttatgctttgaatttcccagagaagacacactgctgcgaaaggaagaaatcgttggtttttctccaactgcttagctgttaagaagcaagaagcctatctccaaggcagcagcaggcctcagagaaaacagtaggacccttccttcaaacaaaggagaaaactcccacaactcaaagcaagtgccccctggtcattctctcaaaaagctacaggctccaaaaaaagacagacttcacaggctagagacgggctctgcactcagggttggtggtcgggggcacagagctggctgggcagggccagaggggaccacattctgccacatgcagagcaaagcagtatctttctattactggtcaatgtaatatgagctgggaatgagcaatttcatacctttttctgagatattaaaaagaaatacgctgatgtggctggaggtaatttcccaaagcctgtggatggagttgatgtcagtaactggctatgaggttgttatttacaggactgtttgatcccactgaagtagtggtgcttaatatgtagctcaatgtggctcaggagcgaggcccaatactgcatataaaccaccatcctgcaatttatccagagagcatgtgggaggtgaaccttgaatcatttgcttgatggatttttttctttccttggaaacagaaaaacgaatctcagttttctccctaagccttaggcaacagggctccttgcaagtcaccttcccttccttgtccccaaaacaaagatgcatacatgttccccgagcactgcaaacacagcctttctttcaggggcagctgggtccctggatccgctttccctcccctgggctggtcccagtgtaaatcatcagatctccaggtccactcgtctgcacacaggctgctttgtaagtgggagaaagaatccacagaactttacactaaatccccatgggaaccaggtgaataggtgtgtgtgtttgcacacaagcccgtatacatggcaaaccaatccaagagatggtgaccgatgggtccataaagactctggctggcaatgctgtcagcttccttcagctgagcgtcagttagttaccctggtagtctcaaatgtctccttgtgtctggagacaacatagggcctggccctgaggactgccaggctgggtcgcccggcagctgcacctgttgtaggctgggtaggccccctaaaatgctttgggcagcccaagagtgtcttcctgagtcatgtggagctgcctcaccagcagcccagtgagagattcacagccctagtggagagtggaagccctgggctatcccaagcaatccttgcgtctgtggggctggtgccgcccctttgcgtctcttcttgcagctaagcagagctcagaggccaggaaaaaagccactgggtgtctttggaaacagctgacctgattttcaacattccctttgagcatttgctaaaggggcaccacagaagggcattaaggatgccaccgtggaaggaagctgcagatgacagaagcgtgagcactgctggaaaatgagaaagctgagttgggactgcgtgccatggagaacctgtactgtcaccctccccagccatctgactaccagcagcttcagcagcatcatcatttgcattcattcatctcttttcctcactccctccctcgatgaatctctttgggaatgatcttcaaaaactttgcttaggtttcaaggtgcttttgattcactgtatttttctttaaataagccttttaattgagcacaacgttcatagagagaagcgcacacatcctgaggggtcagcccaaagaattttcagaaagtgaacacgtgagggtggcaagcacctggactaggaaccagaacaccgccagcgccccagatgtgcccttttagtccacttctagtcactgtgctcccatgagggtgctcaccctaccctctagtactgacattgatgttgcttatttgtgaacttcacttgaatgaactcatgcaacagcgtgtctgtgaggttcatgcacgtttctgaacgtgggatcgctgaagtgtggggttgtattcgaaactgtagtttgcactcaaagaacagcaagtggaagaaaattactttggaaaggaattttcaaatcgagtgtgaatgagagggtctttgggactttggcctttggagttgagctgacctaaaggtagaaggtgaactaggatcctcttagctccccactccactctgaactgtgctcccagggcaatccagcttctgctctcttttactactacttcatgtaatgataaaaattatgtcaggttaaatatcagtaattgcttaagtgagtttgtatatcaagctgtgtagtaacgcctcactgaaacactgctcagctgagactgagattcagtgtgttgacgaaaagagttaaactctaacatatttgaagagatttattctgagccaaatatgagagaccatggcctgtgacacagccctcaggagtcctgaggacatgtgcccaaggtggttgaggtacagcttggttttatgtattttaggaagggatgagacatcaatcaaatacatttaagaaacacattggtttggttcagaaaaggcggaacaactcaaagtggggaggcttccaggctataggtaaatttaaacattttctggttgataattggtagagtttatttgaaggtctggaattaatggaaaggaatgttcaggttaagataaaggactgtggagaccaagttttattgttcagaggaatctctcagatagcagacttcagagagagagcaggttgtaaaatgtttcttataagacctaaaagggtgcctggctcttagttggttatctcctgggtctggaaagaaaggaaggaaaacaaacggggaaggagattctctgtagattgtggatttttcccacagagactttgcaggacaatttcaaggcatggcaaggaaatgtattttggggtaaaacatttcgattttcttccttgttatgccagagtcagattggaaagtaagtcactatatacagggtcaaataaaacccatctgatgagaatttatggtttgtagggtatgactccctagaccccttagataggaatttgggcaagataaaaaaaatcagatcttagtcctcaagtgtaaagcagatcaattgagaatccaaatctgcagggtataaggtcagacgcagtcaaccgatgcatgaaccaagaagcatttatagaagcctctggcgcttttcctttgctcacctctgtccacaggctctgaactaattccttcatttttttttaaatttgcaaataagattactgtattatagatttgaagatgcacccaattattttataagcttttttttatgtaacactaagaaattaaaaaaatgctgaaggtgcagtctagtaggagaccctgcagctgaaatgctgaagtcattctttgtgtgcaggaaacaagaaaaaagcctgccatgcagaaagtcactgagaaacctgctggcctccatggtggcacgtgggggagggaggagaagaaaactcctgaaagaatgtggaccacacagcatgctctggtgggtatgtgggctgacaaagaacagggagcgctaacagtgaaaatgaagagcctgccccgcctcccagcgtggaccttgacctggggccagaccaaggccactttatagaaacgtccctggagcgtgtctcaggtggccctgcctgcatcctggcctacctctggggcccttcttacccacagtcctactttccacccaccttgaccccaagaggtgttcatttataacgcccctgcatcttctcaacttcaaagtcagctcggacctcgttcacccagacaagaccttggctgagtcttgacaaactggcacagggtcccccgacagaagacagctttcctttctctttgctttcctttatcttagccgagtccggcctccccagcactcttttccatctggggacagttcgtgctggtgggaattggcaggggcacacggtccagacagagacaactgaaacacaggtgtcccgctggtgtgccagcagggcggccgtcctaccaggagcaggacactttgttggccactgcccatgcaataatcagcgtgggacaaacagtcccatggggagtgaggtgatgtgaagaaaaggaagccgaagaagagaagaagctgaagggccagtaaacacacacatagacgctcaccctccccttcgttggaaaaatgaacggctgctgttgccgagggctggtgaggatgaggccccaggcctgcaggagggcacatccggttggccgcctcgacggggccatctgggtagctgctgccctttgcatgtaaataaagctcctcaaagccccatttcacaactgctgggagaaacagcatccttccctccttccctatatttccagggaggaggtggctggttttttttgttgttgtgttttttttgtttgtttgttttgttttgttttgttttttccagatttctcccagccacatccagcttcttgttctgtaatttttttttccaaattactgactgccttctttcaaggggaaatggaactcagaaagatgggaagatacatgataagagcacttaagagagaagcctgtccatttccactaaggttgaagtgtgcatactccaggatccagtccttcatgtctacacaaggagtcccaggaaaaggtctgtgcaaaacgtttgtaattgcaacggagcagccacgacctagccctccttcagcagaggaatgaataaactggtttattcagtcaacgcagcagttaaaatgagagaattagtgatgttgaaaggaaaaatgaagagcaaacagatacagtaggaagttatcacgtacatttcaaaaacattcaaaacaaccgtacatatttatggattcatgtaataatgcagtaaaaatacaaaatcagagccaggactgtggcacactgaattcagcacaatggtcgtctctaaatgtgggttcatgtgggaggaggtgaccccggggtgggggcttctgctgtatttgtcatgtttgggttatttgaaagggagagagagcactgtagttatacagtaaaatgcaaacatattttttattttagtggtgagtatacaggtgtctgttgtgctgttttctgtgtgtttgaatggtttcataatttttagaaaattaaaccatgaggtcggatatggtggctcacccctgtaattccagcactttggggggtcagagcagaaggattgcttgaagaccagcctgggcaacatagcaagattctatctttacaatttctaaaacatttttcaatttaaaaatgataaacaatgaaaaactgcaaagaaatagattgaaaagttcacagttgcttttgccacattttgtgaatatgttatttttataatgaaaaa

SEQ ID NO: 3 - DNA, 425 bp, Homo sapiens
  misc_feature 1..425: note = lncFIB #4: ENSG00000251281
  source 1..425: mol_type = other DNA
ttagcgcctcttcaaagtctctgtggcactgcatttggaagcttctaaaggctgcctgcattccttggctcatggcctgcatcattccaactttgcttctgtcatcacatctcttctaactctgacgctcctgcatccctctataacaacgcttgtgattacgttaggtccacctcataatccaaggtcattgttgagagacaaaatgagatcatgtctgggaaaatacttctaaattatcaactccctttctatggaagaggatgaccaaagaaggatgcactccttgtctggaattatgccagaggtcttaaaatgctcagaaatgaaatcagccttcttccctgtcctaagaggctcaggttttcggcaagtaactcataaaattaatgctccactgaaaataaaatctgatttgagtagta

SEQ ID NO: 4 - DNA, 967 bp, Homo sapiens
  misc_feature 1..967: note = lncFIB #5: ENSG00000268941
  source 1..967: mol_type = other DNA
tacaggaagtcaaggtcaggccatacccaccacagttcgggcagccatagaggagagctttcttaacggtccgctgggacccaagtgggcctcccctgggactgcgtgctcccctacagaacctgatggctggaaacactcacactactgcctgtcccggcatctctccctccgccgcaggggctgatgctgccacaggacacagagcagcaggccccagaggcttctctgcacctctgctgcctttctgagtaatcagaggaggccacacccactccaggacacccacacgggccttctgggcgatgacgctcctggcccaggagtggggggctgcctcacacctgctgctgtggggcagagtctctcctgggtcggggcctcgctggcggatctgctccatgtcgtgtgatgtgctgcagccccgcgtcaccgagagcctcttatgggccaccaagtccacacagccactggccatggggcaccctgcagggcctctggacacggagacctgtggttctcagccagtggtgattttgtgtccagggaacacctggccaagtctgggttcaattttgtttgtcacagtttaaggttggggtgtcactagacatcctacagtgcacaggacggccccacagcagaccagcccgactgtcagcagcaccgagccgggaaagcctggtctggagccgtttctctctagggtccctgctagggaacccctcactgccatggagaacccctctcctcctgtctctcaggactctccgagggtcaaggatggatgcctccccatcgagggccaaacaaactgacaaatcgagtgggcaaacggcttactcaaaggccaggagcagctacccgatgactgctccctcgacctctctcttctcactttcttccctattccatcctttgatgggtggcctgttacacgtttggaaataaaaacaagtgggaaaga

SEQ ID NO: 5 - DNA, 1014 bp, Homo sapiens
  misc_feature 1..1014: note = lncFIB #6: ENSG00000262061 - variant 1
  source 1..1014: mol_type = other DNA
ctacatgtccccacccctctcactgctgtagcttcagggcagaagagccacagagccaaatgctgcagagatgaagggagacggaaagagccaaatgctgcagagatcaagggagacggaaggatgtcgcctccagagctggggccacgggcagcgccagggcaggttcatcactgatggcaagtctgtggctgaccacgttcacaggggtggccctgagcaaaggtcggacatgaggacccaggagaattcaggccccggctctgggacggctgtgcacccccaccctggccgtggctggcacctcagaaggcacagctgtccttcactcgcacggagcaacctcgggacagatggtgggagaggaagggcaactcgaggaggcacattttaggaaactgcccacggagccagtttcgattctccaaacctcgtcagcatgcagaggctccctgactcggcagcaaggagggcctcatcagccgacccccatgcccggcacccccttcaccagtggcagggtcctgaaaactccagagagcagtgccctgtgcattaaacacagactgcgcccttgtactcaggaggatgcaattctctctctaaagtcgagagctcaccaccccgggcgttcgtcactgcagacatcactgcagacacagagacggccattagagctcaccaccccgggcgttcgtcactgcagacatcactgcagacacagagaaggccattagagctcaccaccccgggcgttcgtcactgcagacatcactgcagacacagagacggccattatgcagcctcacgtggtcactcctgggggcggtaggatattttatcctaaagagaaaacaccttaaattctcacatcagccacccccatggcgactcttaacaccttaatgtagcaccttccaacttttcctgggcatttatgtacaaattgtaataaaattgggttcttactgcatacgctgcttgctggtttctaaataaaattgggttcttact

SEQ ID NO: 6 - DNA, 1242 bp, Homo sapiens
  misc_feature 1..1242: note = lncFIB #6: ENSG00000262061 - variant 2
  source 1..1242: mol_type = other DNA
tctcactgctgtagcttcagggcagaagagccacagagccaaatgctgcagagatgaagggagacggaaagagccaaatgctgcagagatcaagggagacggaaggatgtcgcctccagagctggggccacgggcagcgccagggcaggttcatcactgatggcaagtctgtggctgaccacgttcacaggggtggccctgagcaaaggtcggacatgaggacccaggagaattcaggccccggctctgggacggctgtgcacccccaccctggccgtggctggcacctcagaaggcacagctgtccttcactcgcacggagcaacctcgggacagatggtgggagaggaagggcaactcgaggaggcacattttaggaaactgcccacggagccagtttcgattctccaaacctcgtcagcatgcagaggctccctgactcggcagcaaggagggcctcatcagccgacccccatgcccggcacccccttcaccagtgggtaaactatgctcgccaccactcagcactcacctcttgggctcagagggcagaaagtctgacccttctctcttaaaagcagggtcctgaaaactccagagagcagtgccctgtgcattaaacacagactgcgcccttgtactcagagagatgtggctttcccagacaaggaagaattcagaaaacagcccagacatgctcagagcagacagctggactctaaccaggctctgcgacttgctgactcacaggccgtttgtccctcaagattccccacggggtagtcggcctcaaggcagctccacctgacggctgagctcgctccttgctgaggggctggtgcccccaggagcctcctctgggaagctgagacctcctggtgccacctcgaccccgccgggcgacacacgctgagcagaggcagcactcgggctggccggcccagcaggcagatgttccggaacccttgtaaaaccccgcagtacagggactaggaggacccgaggggtacagggaacggaacaggagttgccggtgataatttcccgaaaaattcctaggggacagaggctcatcagccccaggcaacttcctgagacagatcggtaaaaacaaccccttcttccaggaggatgcaattctctctctaaagtcgagagctcaccaccccgggcgttcgtcactgcagacatcactgcagacacagagacggccattagagctcaccaccccgggcgttcgtc

SEQ ID NO: 7 - DNA, 870 bp, Homo sapiens
  misc_feature 1..870: note = lncFIB #7: ENSG00000250697
  source 1..870: mol_type = other DNA
gcagggtgcgccagcagggtgcgccagcagggtgcgccagcagggtgcgccagcagggtgcgcgatagcagaagcaggaagagagccgggggaagacacctaccctggccggaagacacgaagatccagaaaaaggccatccgggtacaacgtagcagttacatcggactgtgacacttactgtttacaggagactataaaactttgcccagtcgtcacttggggcccgcctgcacccaggccgtcattaaaacagcatgttgctccacaccgcctcgtgttgtctgttggtgtgctctcagggtggaaacgatacaagaacgttacacttacaggctttagtttatattattctcatagaagacttcctcataatccaatttaaaaaatgagagtttccctgcacaagctcttgctttgcctgatgccatccatgtaagacgtgacttgctcctccttgccttctaccatgattgtgagacttccccagccacgtggaactattatgctccaagacactgaatctgaaaggatgcatccagtcttgccaagtggtttcactaccaaggccagtctccatctatgatagacccaaggaagatcatttacaactagaatctagtcatttaggagattgtgtgctccctggatgggccatctgcactggtgtgaggtttatctcattgaagggaaaaccaaaggaatcttcttgaatatcagcaccaggaagagcagtacaaactggagaagctggggtatgtaggcaatggcttcaacccacgtattatatgtattcaattactcagaagaccctgtactccacttattttggttgaataagcagactgtaaaaccaacaaaaaatagaaa

SEQ ID NO: 8 - DNA, 2568 bp, Homo sapiens
  misc_feature 1..2568: note = lncFIB #8: ENSG00000259518
  source 1..2568: mol_type = other DNA
aaagaatgaaacttccttactgttcactaacctacgtctcaattacctcccaacatgtttgcagcaagtaccctgtcgatggtaggcaagaggaaggtgtgacccacaaagatcaaggaatctaagccggccaaggttaagcatgaatcgaggaaaggaaacagaaatcctcttcctagtcctctttcctctgttttctcctctgggtaccagaattgcgacagagtgggacagctcaggcagctgcaggtcttggccagtgctcttcctcacccaggccatgcagcatcaacacagtcaccgtagaactcctcttaagacaagcaaaggaactttccaacccagttgagtgcacttttcccaagtgtgtggtaaaaagttaaatgacactttgtgaaagggatccttaagctacaaaaacattttcccccatcacactaagcacttggttttgtccttgtagttgcaaaaggggctggagtcatttgatgaattctgaatgttgtaaggctgcttttctggaagggtaaacagggaaccttttagatgccaagaccttttagaataacgacccttcagaggaaactgaagtgcaaaggattgaagtaaccttccagaaggtacacagctggtgagacaaagcctgtactgggtcccaagcctgcctgactccggactcccacaccccacagtgataccagaatcaccctgaaaatgggaaacatgctgcagctgctgtcctgattccagcctctgaccaggagagctggattgaccaggaagccaatgaagctaagtttcagggccccctgtctgcctaaggcctctcccaaggccctgcaccaaagtttctatttgtgactttgttttcttagaaaaaactcctcaattatgtgaattcacctctgctgacaagtaactccttagaggacgtgagttttagaaggacaagctccaaaagcatctctagatagcctccagaaccctgctggtgtgactctttttggaactagttctaaaggctggaaagtccaagaccaagacaccagcagattcagtgtctggtgagagcccactctctgattcacagaaggcttcttactgtgttgaaaagacatgactcaacccacccatcacttagcagccgtctgggttatcagattgactgtcatagcggcacagtgcttgtgttcaagcaacccttattttacttcccgatggccccaaagtgcaagagtagcgacgctggtgattccgacatgccagaaagaggctgcaaagtgcttcctgtaagcgaaaagctgaaagttctcaatgtaataaggaaagaaaaagaaatcatatgctgaggttgctaagatctatggtactcagggagttttgagattttccaagatgccatcgaggaactgaaaagtgctgcccagctgcagtgagggctttttaaaggcagtttcatgtctacaccccactgagttcagactgtccaatgaagcactgacattgttacacatgcgtgttgtggacaacttgagaaaacacttgtctgcaggaagatgaaattaaaatcaagtgtgatcctaccaccgaaagataagtgttactgatattttttgttaataacattccagttttttatgtagttttggggtagcttattattttcatgtgatttcaaacttggagaaagtttccaagatgagtccaaggagctctcatatgttcctcacccagatccaccaactggccacattttgttccatttgctttattattccttctacctctctctctctaaagtattattatatgttaaatatatcttaatataattatagttatgttaaatatatcttaatatagttacaaataattatgttaaatatatcttaatataattatgttaaatgatgttatgaatattaaaaattataatttaaataatattctaatataggcagacatttagtttctgaaccatttgggaccatgctgaaaacatcattttcctttacaccattttccttcagtgccagaggggaacagagcagtgagaggttgtggggggatcctgggccaggagcatccccatttcctggaataagatagttctcttaaataactacagtaccaacatagaaatcaggaaatttagcattgacactattaactaatgcacagcccataaacaaatcatatcaattgttccaataatttgtgtttgtttgtttgttttgtttttttagacggagtttcactcttgctgcccaggctggagtgcaatgactcaatctcagctcactgcaacttccacctcccagagtgtctcccaggctagagtgaagatttcagctcaatgcagcctggacctcctgggctcaagcgaaccttctggcctcaggccccctgagtagctgggactacagacgtatgccaccatgccctgctaattttttgtatagacagggtttcaccatgttactcaggctgtagaaacattttaatagctgcataatttccatgatgcacaccgtgtttactaattataactcctatggttttgtcaaa

SEQ ID NO: 9 - DNA, 2347 bp, Homo sapiens
  misc_feature 1..2347: note = lncFIB #9: ENSG00000234840
  source 1..2347: mol_type = other DNA
cttttattttctgttcctctaagggaagagagaccaaagagaattctactttgaccctggcttaagagtggaacatagatgtctataaaactatgtggtaccagcatgttatgaagaataagcaagaagaaagagaaggagaagaagcaggaggaggaggagagaaggaaagggaggaggaggaggagaaaaagatggtggtagaggaggaggagaagaagaagaaggaggagaaggagaaggagaagaaggagcaggaggaggaggaagaagaagaagaggagaagggaactcacatgctacctgtgtgagtcacatgtcagtgcaatttctggaggtcaattgtagccctgaagtctcagaacaatggcaaaaagaaatacggagggcatgatacatttggcagactaaaattctcaagattcctatgcagtcgagttttaccatatgactgagttccaacaaagaaatatgagtagaagattgtataagcatcatttttgacccagggctttaagaagcagactatggaaatgatgttagacaaaaagcaaatttgagcaattttgttattcgggttcaaaatgggtcgtaaagcagcagagacaactctcaaaacatcagcaacacatttggcgcgggaactgctaatgaacgtacagtggagtggtggttcaagaagttttgcgaaggaaacgagaggcttgaagacgaagagtgtagtggccagccattggaagtagacaacgacaaattgagagcaatcattgaagctgatcctcttacaactacaggagaagttgccaaggaactcaccatcaatcattctatagtagtttggcatttgaagcagatcggaaaggtgaaaaagcttgataagtgaatgcctcatgaacagagcaaaaattttaaaacattgttgttttgaagtgacgtcttctcttattttacacaacaatgaaccatttctggatcgaattatgacatgtgatgaaaagtggattttatatgacaagctgcaaaaaccagctcagtggttggatggagaagaagcttcaaagcacttcccaaagctgaacttgcaccaacaaaagctcatggtcacaatttggtggtctgccgccagcctgatctactacagctttcggaatcccagcgaaaccattatgtctgagaaatatgctcagcaaatcgatgagatgtgccaaaaacttcaaggcctgcagccggcattggtcaaccaaaaggatccaattcttctctaccacaacgcctgaccacatgtcacataaccaacccttcaaaagttgaataaattggtctacaaaattgccttatctgccatattcacctgacctctcaccaaccaactaccacttcttcagtttgtctcttccagaatcaaagctataaaactacaaatggttctccaaatggagccccagatgcagtccatgactaagatctactgcggacctctggaccggcctgctagcccagctcccacattaatgacatcaaaggcacccctcccgaggaaatctcaactgcatgacacctactacgccccaattcagcaggaagcagttagagtggtcgttggccaacctccccaacagcacttgggttttctgttgagatgggggactgagagacaggactagctggatttcctaggccaactaagaatccctaagcctagctgggaaggtgactacacccacctttaaccactaggcttgcaacttagctcacacccgaccaatcaggtagtaaagagagcttgctaaaatgctaattaggcaaaaacaggaggtaaagaaatagccagtcatctatcgcctgacagcacaaggggcgggacaatgatcaggatataaactcaggcattcaagccagcaatggctacccactttgggtcccctcccattttatgggagctctgttttcactctattaaatctttcaactaccaaaaaaaaaaaaaaagaaaagaaaaacaattaacttttttatatatcagttaaattcatccatgatgttttttatgttttctgtattcttactggtcatttttctatttgttctatcaagtatccaggaatatgtgttaaagatgtccacttcaattgtgaatttcttgagttgttctttcagttttgttctttttttaccttatgcattttgaagttgtattatttcctgcatacaaatgtagaattgtggtgtcattcttttggagtaaaccatgatcattactggcatgacttacttacttactatctatctagacatgttgattgcttcaatttctactttaatcttaataaaaatacaccaacttttt

SEQ ID NO: 10 - DNA, 2229 bp, Homo sapiens
  misc_feature 1..2229: note = lncFIB #12: ENSG00000233901
  source 1..2229: mol_type = other DNA
gtcacacgcggcgccccattaaagcggccgtcccgggcgcgcgcctcattcgaggcgacgggctccgggccctgatagatggggtgcggggacggagacaaatgacggcctttgggctcggaatacccacctttctggtaatgcagcccagcgggtcccagcctcgttttccagccctcactcaaaatggagtcgctctggttcgaacgcctctgacaagtgtgtacctacgtgtcaggcccatccttcctgcaggcctttgtcttggtgtcaccctgcctgagctggccccaaccacagtagcaacagtgatgctgatgtgtgactgctgccaggagcctgctcacatgaccgtgtggagaaagttctttccctgaggaccatctggagtggacgcgtgcactacccctctctgaatacaccctccccacgaggccctctggagcatcctgtagagccagcccctacccagagaccctgagcccagagaatgcgctgggtggtgagcctggggacgaatgcagagccctaggtctcctctccagaaagaccggattttgccatgttggccaggctggtcttgaactcctgacctcaagtgatccacctgcctcggcctcccaaagtgcagggattacaggtgtgagccactgcacccggcctcaggcaggtatttttaccagccgaatactcttgcactgtgcactcttcgtgttcatcatcagtcccccggatgggtgtttaaggcatttccaaagctctgttattaaaaacacgatgcaatgagtttctttcagcatttccccttgttgccgtgtgaatgcgtccgtggtataacttcctaaaagtggaatcgcttggtcaaagggtgggtgcatttagcaacctgatggctttccaggagacaagtgctgcctcccggccacgagcttttaagaagaactgttgatggaacccgagctggagtcccagggaccggggcgagttctccatcccaggggattttcagatgggacactggggagaagctgccctgcccatctgaactctatgatcccagtactaacactggtagctgtgccatccccagtatagagacagcaccgtgctcttgggagggtgagtcattggcccccggctgcccagcaaacagcatgtgacccccacttcccagccaggcctgggtcctcttgggttccgagatcccagccccctgctgtgatcctcagggatcccttcttgactggcgtctcccagaggcacttgtgttcccccagagcttgctctccacaagtttctgaaactcattgctgctgctggagactccattgggtctagctcttggctgctctggttgtgcaatcacccgtctgcacgtttttctttcgtgcagagaaagatctgcctcatctttctgcaagagcatcttggtatttatacactttttgtcttgattttacaatttttggctgggcacggtggcttacgcctgtaatcccaacactttgggaggctgaggagggtggatcatgaggtcaggagttcaagaccagcctggccaagatggtgaaaccctgtctctactaaaaatacaaaaattagcctggcgtggtggcgggcacctgtaatcccagctactcgggaggctgagacagagaattgcttgaacccgggaggcggaggttgcagtgagctgagatcacaccactgtgctccagcctgggcaacagagtgagactccggctcgagaaaaaaaaattatttcttggttgggcaatgtcactgttgtttctgaaaaaatacacccttattgtaccataaactgatgagttgaagcaacactggaaactacaagaaagaaggtaaaagtcatcaacacgcttcacctggagatgactctggttaatgtctaggtggacgtccttctgcagagtgtacacatttcgccccatcccctctaatgacaatgaaaacctctctgtttcttgctgaaggtggggtgtgtaagtcactggagtggatctgagggcctgaaaatctccatcccctcccctcccacagcgtaaacaaacagcctcccagcacccctcccgcactgccaggttggaagccaccgctccagctccagggagcaaggcttggtgcccactaagcaggtgctaatgattcccccactctcaaagaaagttccattcactccccaaggttccactcccctaccttccagac

SEQ ID NO: 11 - DNA, 1623 bp, Homo sapiens
  misc_feature 1..1623: note = lncFIB #13: ENSG00000248187
  source 1..1623: mol_type = other DNA
ggcatgggaggctcagcaggtctgttcccttggcatgtgggacatcttgcagtggatgcagggagggaccatgcctctagcattctctgggaagaataaaggagaagtttatcttcccacctacctcccattgcctgtttttcattggtcaaaacttgtcccactaggaattaactccttcacatttccaagtttatatcatatgactgctccaactacatgggaccccaaaccccaaacctgatgtatgtatagtgttcctgtttcatccaagcctggaaatgaggggaagagccagaacctctaggcttggtgctctggaaaaagaattagaattgcattcaaatagtcaggcttcaagttgcttctgcctctgtgtgaagcaaatagtaacaactcacttcgaaactcaggctcctggactgtaaatgaggataatgatactcaccctaccaacctcagggactgttctaagagcaaaatgaaatgagactgagctctgaaaatgcttaaatgcttttcaaagtagagtgatcgtgctttctgtagcatcaaaacagtcctctataaatgggcaaagtcaaggcatggactggatctgacaacagagaatggcttgagggctggaaaaagaaaaaaatgcaaatgccccaaatctctgcttcccttacagtgtggaaatcgccccataacacactgcaccaggtagggttaaattgatcccaaatgtttcaggaatttttaatgcccttcgaagagacatttccaattcttcatctgcccaaagtaatgctgctaacctactatctgtcagtttcctcagtgctttgaagatttttctaaagtcatctgacttggtttccctttgatttcaaattcctgcttattatatgtatgaccttgagcctttttaactcatctgagcctctctgtttcctcatctataaattgtgaataagaagacgttactgtaggattattagaaactttagaaataatacttttaataccagcatagtgcctgatccataggcatccaggacatgccagctaagatcattgattattgttgtcgtttttagtatcatacagtagaaagaacaacatctttgaaaccagacaatcccgcatttaaatcagctctgtcacttacgaaacgtgtaatcctgaataactgaagaaaactttttgaagttcaattttgagataatgatggatgatttcagactttgtgagaatttcagaggtatcggcagtaggttgaatggtgaaccctccaaaagatatatctatgttccaaactcagaatctgtgaatgtgacattatttgggaaaagggggttttgcagatataattaagttaagcatcctgagatgagatcatcctggattttctagatagggcctaaagccaataatgagtgtctaagacagaagaagacacaaagaagaagagacgggaatgtggtcacagaggcagagactggagcaatgcagccacaagccaaggaacacctggacccaccagaagctggaagaggaaagaagggatttctttttcttctagagccttgggagggagcatggccctgctgacaacttattttcagacttctagattct

SEQ ID NO: 12 - DNA, 3249 bp, Homo sapiens
  misc_feature 1..3249: note = lncFIB #14: ENSG00000251615
  source 1..3249: mol_type = other DNA
tatgttgatgaagcctccggatggcaggcttcagagagaatagatgtgcatgtctcttataggcccttaaaaggtgtcagagtctccagaaaaagcctaataagggaagcagattctctgcagaatgcacatttcccccatgagagacagctttgcaagggccatttcaacatatgtcaaggaaatatattttgaggtaaaatactttgattccttcggggctactatgtgtcatgtgatgctataccggagtcccgttgaattgggtatcttattgctacagagagtctgttttgtcagtctaggatctctgtttttattttaatgttggtcagttgtgtctaaattccagagcggggcaggtataatggggcatcccgagccctccttcccattcatggcctgaactggtttttcaggtttctttgggtcccggtggctgcaaggggatgcactcagttggctgggggacttagaattttagttttggtttacagcaggatttgatgggcatgcatatttatcaatgtgaaagtgttgtctgtgggcggcggagggcttgggccgtgtccaggccacgcactgcaagggtgcagcccccacacacggtccctgctcctaaagctgttgcccccactgcaagagtggagtaggttgaggtgtagccatgacattctggaaactacattttttgtaatagtcattttgaaaataaaacatttgaaaatactgtgcagcaaaaacttgaataattggaataaaaatgttcagtttaatgtcattgcttgctgccaccgttttctgattgtaaaactaacgtgattattaggtactattttggtaatgtagaaaactttaaagatgaaaatagatatcacttataactccacatggagaaatagttgcccctccaatttaattgatttacttttgggccacaggggcccagaatttgattcattgtactccttggttcatgccagtgttcctgcacccagcactgaagctgaacattgacagtgatttgcccctgccccagagtcctccctgcattcccctcgagggcccccgtctcctggccctggtgtccacttttctctcagctgctgggaacgtgttagtgtgcactgttctcctttccctcacctgctggcttcatgttagcatgcaccgttctgctcactttcttcccctcacctgccgggatcatcttagcatgcaccattctgctcactttcttcctctcacctgccaggatcatgtaagcgtgcaccgttctccacactttcttcctctcacctgccgggatcacgttagcatgcaccattctgcacacttccttcccctcatctgccagggtcatcttagcatgcaccattctgctcacttcaccttcttcttctcgcctgctgggatcatgttagtgtgcactgttctgcacactttcttcctctcccctgctgggatcatgttagtgtgcaccgttctgcacactttcttcctctcacctgctgggatcatcttagcatgcaccattctgctcactttcttcctgtcacctgctgggatcatgttggcgtgcaccattctgcacactttcttcctctcgcctgctgggatcatgttagcgtgcacagttctgcatacttgcttcccctcacctgccaggaacaggttagtgttccccattctgcacactttcttcctctcacctgccgggatcatgttagcgtacaccattctgcacactttcttcctctgtgtacgtgtgtgtggttttcctttcagaaatgggtcatactctgtggacatctttttccacttaaagtgatggcacagccattttcccagtcaagtaacatgaatgtacaagtttagcaaacttcctcctgagagatggagagatcaaggcttctgctccgtggcagccatccgtgcacatgcatcactgtgcgtgagttggtcccagtggcttgctgagacatgctctgcacaggggagatttcttataccagcagtgtgggaggcagtggggcaggtagagccacaagtggggatgagggagcagagtctagggcaggctcctggatggacaccagctacactgtctattggatgtgggacttccctcactgatgaaatgaataagagaatcttcctcatcctgctgttgttggcgttaaaacgggttaatgcatgaaagcaacaaagcccggcctgcagacagcacatggtaagtctcctcttcccagcatgttcaacaggaagccctcattccctctagaatgtaggagtcttgccagcagtggcctgtttgttctctgcctgttgccgacctgatgagaacataaaattggtgtcttctgtatagatttccttcttattgcccctactgtggctgaacataccttcccccgtgcttgaagtctgttcatatatgttctgctgtgataactgcccattcttgttctctatctaagaaggcatttagttttgaaaatattttgttggcttgtaagatatttgtacatattaagcacagcaaacctgtaacatattcactgtaaatcattattcctcatgcatcatttgccatttaactgtatttttttcctttttgatgtaacctgctatagtttacatttttaaatcagaaatgcctgcacattttaaaacgaagactcttatgctctagcctttgaggtcatgcttggaaagtcctttctcacacaaagattatttacactttcatctctttttctctgagtacgtttcagtgtttaattgaccaggaatgtatttcgaggtgtgatatgagacaaggatctgaaatgctttgcccactgaatcatcatgcactgaccgaaatgccacctgtatgagtgctaaaatcattgtcgctggacctgtgtctgcactgtttaattttagtgttgtttctcacatcagcttctgttgttttcatggttgtggttctcatacttttaatatctgttggagcaaataaagcctcgtttctcatcttttccaaaaatgtcttatgtatgtgaactttggtgttatttgtcaagtctgggcattctcccacccccatattaaagaatgctctctggagtttgtctggaggttttgtttaacttttagattcacatcaggagaatctttgtcttcatggtaagtggtcttcccatctaagactatttcttcaagctgtttgtaataaaaagctgtgtgatttttgtatgttta

SEQ ID NO: 13 - DNA, 8398 bp, Homo sapiens
  misc_feature 1..8398: note = lncFIB #15: ENSG00000249669 - variant 1
  source 1..8398: mol_type = other DNA
tggccagggagccgcaggcaagggactaaggggaggggggctcagtgccagctgcttaaaaatgcccctgtggcagcgaggggcaccagaggctgggtctaattagttgagaagcagtgacacccccaaccactccccaaacaggctggctcccgtctccaggccccaaggagccacacctggaccagaccccaggaaagacatgtggtggaaaccctacggccctggagcccaaggagagcaacggctgtaacaagtgcacaggtttgagtgcgctgagacaggggaaccgtgggcaaagaggtccctccagaaagagctgggtgtgagcatgggttcgtgagcttgcctggagccctggcctcagccctctctcactgaggtgccatctggctcgcagatgtggcccattcccagcccctcgtgtttcttggaaatgagaaagtccaggaaagggcacaggggctttcgcaacgaccgtggcgatgatagctttgacgtgtggtcatccagccggccactgcccacacccaagagaaagctggattgagcatcaggagcctgggttcaagtctgcctctcaagctgttccacaggtgaccctgggcaaatcgcacaggctcctggaacttcagaaaggggaagaattattcctgcctctggcgacatcagcgaatgtgccttgagttacgggagtgttttgaaggctgaaagctttgtgtacacggaaggggtcaaagacagccacaagcctgtccctttggtcttggtcatcatgttaaccccaggtttcgatttaatcctacatgctcctttcttatgtgtgaggctgaggtgggggcattggtcagaggagcctctgggaaatttcctttcaggacagttcttttgttaaggggcctgggtgtgctgagaagggaaggtggctcctcctgctcttggagcttggagtttctcacaagctctcccttaaaagccaggcaggccaagaagggataatggctgctcagggcccaaggggcaggatctctgaggcccggaagatctcagtaggtttagcccaagagtatccagaagtggcccaggcccattctccatccctcagagcctggcagcctggtcaggaccctcaaaggccatgaccgtacccgtagggcaccctggtgtccttctgacaggtgggatctttgtttcttacaggcaagggcgctagctggtgggagccaccccgccatgctgatgtcagagaagcaagaactctggagaagcagcctcctgggaccagaggagggccagcagcaggcagcccggagacagaactgtgagcccaccccacctccacccttccggcacccacatccgctccccaccccagcccacctgcaggtggctcctggagtatcctgggtccctcaggataaactccccaccctaacctcagaatgaccaagaaactgggcctgaggatgacaagaagcagaatgggccttggtaaaggaagaccccccactgattggtggcagtgcgggcagttcagggctgtcagcgtctgggttgtaggtaaaaagaaatggaacccaagaagggaaatccaactaggagccaagaatccggtctaccccatcctccttacgaaatagcaatgcaaagcccgtgtgatttggacataaagcctctgttactcaacttgattcggtgaataacagacttgaaagaaatgtggaaaataatagtaatcttccctagaaaggagtaaacagggttgagctcccaaagcaggaagacctggggctgtgtgatcttggacgagtcactcaacctctctgagcctagtgataaagagccagctcatagggctttgagaggatttgtgaggttgggctggtgtccccaggcacagcccttggcacatagtgaaactcaagaaaacagagcagtttttaccattcatcacattattcccacacttgccaattttttaagttcatgcacatggtggaacaggtagggtgaggtgccagctccataagatgagtggagaaactgaggcctggagaggtgaaagcattcactcagagttgcatgtagagtcagttgcaaaggagaattctggcaccagttctcatgtggatggaaaaagtgcaggaagatttggaggtgagcagaggagaagggaggaggaggccgggttccaggaaaggagctgacatccagggatgtgggacaaggcccaggcctgtcttacataacatggtccacctgttcacccctgcaggccccacaaatgtgtgcaaacgtgcaccccggcacagcgcacacccagtcctccctcactggcaaataagccacagagactgggtttaaacaaattggattaggagggctacaggcgcctcctccccccgactgtccggctccgacactaaacagacctgcagaactgccagctgtggtctctcggacacactgacagtcggtccttggctccctcaccccacccgtgggcctcaggaagggggttggggtctgagaggatgccacagtgatggatggggtgagggaggttccagggctagctcaacagcgcacaggatgttagtgcttttgcgaggcacatttccgagctgttcccagacccgcccccacttcccccctcaccctcccctccggcagcctctcccatggccactgctgcgggctgtgagcagagccccgtccttgagcagattgacctcatctttcagtcctgcagctgggctgagcggggagtcagctcagaagacactggcctggggatgagactcggggcatgcttcagagggaagaaagagcttctgaacagacaatgctgtcttaaaggtggcatcttggtgctttctcatgccttgttttctttcatcctcagaatgtgtctgggggtgagaggacgggtgtgactgctgaaacttcctttcttggtgattccacatcactcctttctgatccctgagcctgtgccacgccctgtgtgatgtgccggggacaccagggtgaatacttacaggccctgttccccttccttcatgaaacagcctccttcgggctgggtcactgggcactggggcctccctggtcttccaggattgcaacttcacagaatcccttttccataaggcacatgaaagtgactgtgacccgcaacgggagccttgacaaccagccaattcagaacacagaccttgacatcggaaggccccgagttcaaatcctgccttgctccctttcagctggaaagctgtggtcaaattacttagcctctccaagccagttttcccttctgcaaaatgaggttacacaagctgccacctcacagaatgagtgtgagaattaattcttattttccacaccctcatttgacagggggagaaacagaagcccagagagggaaagggcttggttaggatcacaccatgagttgggggttaagtcaggatggggtaggtcccctctttctctgggtgttcctctcccccatcccttatctcaacataaatcctccccagttgcccaggttgtccaggcgattcctggcacacgtcctggggcctgccctccacctttagccctgctctggcttctgcagcagcagagtcctgggtgaccagcagtggcccctggccctctgagtggtggtggagggtgggggtgcactgcagcaagctcccagcccagagcagcctggccatataagggaaggagctcagggaggcctcctggctcaggcctggcgagaaaacccagacagccactgtttattttctcctccccagctcacccacgcctctccaagcctcccaacagaagacagaggtcccccacagccagagacatttcctgaagacatggggaacacagaggcagaaacagcccatccacccaggagctgtcccccacactgccgggagccggcacccagagccgccaggtaaaactgaggccacctggttcaacatcacctttcacagaaggggaagcagccacagaaagaagggcctcgttaagaagtggaacctgggacccccaagcggtgtctctcatcctgactggggatccagagtaggagggagcctttggtggggtaagtggaatggggcggggtggggggtggccatagacccctcttctcagtaaggccctcatgtgaaggaggcaggggttgggacaagtgctaagtatgcaagactcaagggaagagctgctggagccaggagaagcacctccctcccggcccctctgcccctcctcatagcccagctgcactgactcctcctccaggaagccttctcagcttccccaggggtgggaacctttttgtcctccaggtgtgcttggctgtcctttcttgggctctctctctctctctctcctcatcccacttgagtctgccccctattcaccttgtgaggggaattttccttctactcaatctgaccgaggtcctccaggtcaaggacagcgaggctctcagtcccacttccccttggcacatagaagaggcagtgcgctgaagggacaggtgaaatgattagaccctgcccccaaaccaaggcctggccaattggacagggcatgagacattcagcgtagaggttaaaacgagggccctgggttaggaaccccagctcagttctcagctctgtacccttggaaaattcccttcccatggagctttgtggatgcacaaggacttgcacaaagaaaacattcaatatccaggactataaaattccacaaatgattgtgcttattacattcattatcacgatgattattccagacacaaaggaacagaacgaggcaccaacagcaaggggcaagcagattcaagggccacagaggagatggaggcaaacaccttcccctggtcagaggctgtgcctcagcccttctccctgcatcagtttctccttcagaagcatgggactacctcccatctagttctcgtttctaaacctaggggagatgctatctttgctgcaataatcttagcctacatcttggaatggaaatggccttggtggaaatggtcttcaactcctctggtccaagctcaggccctgtgaccctggaacaatccccttcctggtcctccatgtaggagcaataacattcccttgccagcagcaccagccattctgatgattaaatggtatcggactctgttttccaaactcagtcattcagatgccccctattttatttcttccatgtctgcaaatgattataatatttttaaatgtaggatgagtcctttttattacacatagaaatagctactgtaaatagcaaactctaacactgtgcctaattaggaaataaaggtaaccataaatacagtaaaaatgaaacaatgttattatggtttaacctgatagtgtggcttgcaaggccctgggcctgaagcctgggcaataagtgagagttagaaaggtgtcaaagacatgatagcagcaaactgaggctttgtaccccacggtaaataggactgaaagcaaattcacagggagcaactgatccattccacaacagaatgctccctgtcaattcgctttccattctgttgtgtcctgtctcccagcagagactacaaactccccaaaaccacttacccaccagctgcacgtgagaagccaaaggtagtttatgtgaaagggctttggaaataatcacgcaccaagtgaaggcagaggacacaccttgtcagcttagttctcagcagcaaatcatctcttttccaggataaccctccctgattcttattgaaatctctttgctgaccacactaagctcttctctctcaggggcagtgggagccgtggagagtggaatagaccagctgtctgtgacctgcgagggagtccaatgtcggaatcactccccagccaaatgcacggttttaaaaaatctatttatttatttatgtagagaccaggctatgagactggctaatttttcgtatttttggatagagacaggatttcatcgtgttgcccaggctggtcttgaactcctgggctcaagcgatccacctgcctcggcttcccaaagtgctcaggattacaggcgtgagccactgtgcccagccaccaaatgcagttgaaaagagtttctgcaagataattccacagaagaggaaagcagttatctggctgggaataccttagacagaggctgtcctctacacaggctgtcagaactgacctactgacctgcctgctggcatgatagaccagagggaaacactctttccactcttcccagactgagtgtaagagacagacttttttttttttttttgagacggagtctcactctgtcacccaggctggagtgcaatggcaagatctcggctcactgcaacctccacctccccgggttcaaaccattctcctgcctcagcctcccaggtagctgggattacaggctcccgccaccacgcctggctaatttttgtatttttagtagagacagggtttcaccatgttggccgggctggtctcgaactcccaacctcaggtgatccgcctaccttggcctcccaaagggttgggattacaggtgtaagccactgctcccagcctttttttttttttttattagagacaagggctccccatgttgcccaggctgatcttgaactctaggctcaagcgatcctcctgcctgagcctcccaaagtgctaggattacaggcatgagcactgctcccagctctgtttttgtttttttaaggaggtatattttttctgttctctctcagccctgaagaaaggctacccatccccttcagacattcctgaaaatatatgtcatccaacatcaaggccaccacagtgaccactaagcaatgcaagaaggtcctctggggtgccctgagatcttggcagggcaggaagagtgagaaggggctttgcctgctcacctgaaagttcccacccaagccgggcccagaaaacaaaatgaattatctacatggtcatccaatcagccaccaaatggcattacctaggtccatcatcatgtaccaagccacaggtcagacccagggccccccaaattaacaagacacaggcactgtcctcaggaagtggagatctagccaggtcagacccacacacaaataacactgatactagagataatgagattgggaattatgattcagccattcaacctgggcttatgtggcatctacaccacctggccctgatttcagtagtgagcacagttgacagggtcccttcccttcacaggtgagccatgtagtccacaagtggaggtggtcgtaagtgaaaaccacattaccagcatggtaaagggttagaaagagggcaccgggtgggcgagaacatgaagcagggggtgctgatggctaaagaagtaaaggaggggctgggcactgtggttcatgcctataatcccagcactttgggagaccaaggcgggtggatcacctgaggtcaggagttctagaccagcctgggcaacgtggtgaaaccctgtctctactaaaaatacaaaaattagccaggcatggtggtgcacgcctgtagtcccagctacttgggaggctggggcatgagaattgctttaacccggaaggtggagtttgcagtgagccgagatcgtgtcactgcactccagcctgggcgacagagggagactctgtctcaaaaaaaaaaaaaagtcaaggagggtttcccagagtggccacttgattagagacctagcacaggaggaagagatgggcagggagagtgacggggagcagcacagtccctgggagcccgaagtgggtgggcacagggctccctaggagaatggaaggacatctatgagctgtagcccaagaggaagaggtcactggggctagatgcggcagaccctcgcaggctttgggaagggcttcagaattcagcctgagggcaatggggagcccttttgggatattaaacttgagtaagatatgagcatatttgcatcttgaaaaatcattatgggaagatggctgggaagagaggaggagtggcagaagaaagataggttggagacaattgattgctcgatgatataaaatgttaagtaccatgaatgatgctgttaggctggaatgcgccaagcataaaggtggggcatggcatcaaaaggtaggtcaacatattaaataattccatgtattgaaatatccagaaaatatatagacagatctatagagatagaaactggtctgcccaggactaggggttgtctaaggataaggagcttcttttttggatggtgaaataacctaaaatatattgtgccattgtttgcacaactttgtgaatatattaaaaacctgttaattgtactcactaaa

SEQ ID NO: 14 - DNA, 5524 bp, Homo sapiens
  misc_feature 1..5524: note = lncFIB #15: ENSG00000249669 - variant 2
  source 1..5524: mol_type = other DNA
atgatctctaagcatccatccagctgatcggctctagttctatggtcctgttggcttctaggattccttgttgttgtagtcaattgggggaagaaggtgcagagggagtgcacagagttaacatcctatcagcccaagcttcacctcggcacccgagtctcaggcagtctccctggcttctacataggcagtgcttcttcctcattgtgtggggctttgattttgtaattccaagagcctggggctcctggcaaggaaaatggttttcaaataatggtttcgagaaacaaagctggggaagaggcaatgtaagctcaggctctggcaggcaggcagagatcctgggaaggctgggtgctgactgcacatggagcaatgggaagggatgctggtgagaggagacgggggcacttaagctccggccccagctctgctctcagtgcccggctctgtggtcttgggctggccccctcccttctctgggccatagttttcccatctgtatagcaaggccattggacaaaatggtccctctgcagatgtggcttctgagttgtttgtgcctgagggacagccagtgttgggaagttcccccaggaggtccctgagccgagtctgaactttgaccacaagcttggagtccaagcagatgaagtcctgtgggagcttttggaggttgagcctgagtgagggagagtagctgaaggttctgtgactgaaggcttggccagaggggtgccccgagccctccagatgaacttggctgcaaccagcctctggtggggaaaggactgatctctggattcaaccacacaggaatgtgggacatggaagtaggtaagggatggaaaagatggcagagggcttcgcgggatgaagcagtggggccaggggacttagaggaatgcaggaggcttgtgatgggaggcagggctgggtagaggcaggggcttaggattggaacttgaagatgtacagacagcatggagtcgggctcctctgaaaacactctggccacatccggagcccagaacagaacagtcctctagcaccggcctctgtcttgtaccctccaccttcccgcttcttgtcacacaagacccaaggccatcatggttcagaaggaggctctgaattcaactgcctgggtccaattctggcttgtttacttactggacaagtgaccctgggcaagttgcttgctgtttgagcctcagcttcctcctctgtaaaatgggtacaattctgagcttgcatggttgtcatgaggagtgagggatgtaggcacatagagcaggatgaatggggctgatgttacatcgcagtcagagcccacacctcctgcgggcaagataccctgagctatgttgagggagaagtgggaatgaaacccggccagggaatgcccagagttgctgaagagctctggaacaggctctggaaagaggcaggaggaatcaaaagtcagaggctgtgggacacaggaaagtgatcagcttgagatgcctgaaggactgggggggatctcctttcctgcctttctagggcattgtgtgggcaatgtatctgaaccactgtgcactcacccactgacgggggaccccaagtgaggcctaggaatctgcattacaagcaccccatgaattcccatgcatgtggaagtttgcgaaatgccaggctgtagggcggcctaggactctcacaaactgccgaggcaacggaatccacagagagaaagcactgctttaggttatttagcgagctgatggcagaggtggaacagaacctgcctctctgcccagccagggattccataaggtggtgcaaatcaggagaaatagatgacactatttgtggagttcttatgaggtccaggcactacctcagatcttcacatgaactaattcatttaatcctcacaagagccagtgaggaaggggcaattattatccccactccacagatgaggtacctgaggcaaagagagtttaggtggcttgcctgaggtcacacagctcatgagttgttaagttgtgtgtgccagctgcccctggggctgctaactcccccaggagtctcccacctcctgccctgcctcttagctacctcaaaacttcctggagaccctccaacagacctcatggaagggggcagaatatgtatgggagacttctgggagtcagacactgtgctgaacagcttgcattatcatttaatcctcccaggattcctgtgaggcaggaatcagcatcattccatcaccctcactttctagagaaggaaaccactgcagattacccaatgtcacgcaattaaaaagtggtgaaggggatttgaacctagtctatgcatctgcagaacgcacactcttgggctgcccaccccgacacctctgagggcagtgatgaagaatcccacctcacagaggagacggaggccaggagtgaggccctgccggagcctgagcccaagccttctagctctgaggccactgctctcccttcaaccctgttgctgccccgcaacagaaagtttgtcattggtccctcacagccacaccacagccctttgggcaaaatcagccccttcccagcctggccagttctgggggaaaatgacacctgacacctgacacctatccatttttttttttttttttgaaatgaggtctccctctgtcaaccaggctggagtgcagtgactcttctcaattgactgcaacctctgcttcccaggctcaagtgatccttccacctcagcctcccaagtagctgggattacagatgtgtgccacatctggctaattttttgtgtttttttgtagagacagggtttcgccatgttatccaggctggcctcaaactcctgggctcaagtgatcccccagcctcagcctcccaaagtgctaggattacaggcatgggccactgcactcagccaacacctatccttgaggaatagaaagatccaggctccacaccacacaccatcactgactcaagtggctgttctgattcccagctgagcctgaggggttcggggaggtaatctctgaggtcctcactgctgggccgtgcctgggcatggcctcttcctgcaattttccaactaaactctccgggggggctcagcgccatggggtggttcgaagaaccatgatgaaggctggttcgaattgtgatgaccatttttgtccacatctcctaggacccataagccagagtttctctggagcttatagctagaaggggttctgggtcctggagtgcaggcctgtcaactttacaggagagcactagattgctttctgaagtggctgaaccaggttatgcttccatcagctgtgtatgagcatccccatcttcttgaccacacttgaagccatcagtttccttgaagcatatgggttgcacacttcattttgcatgtatcaagtttatataataaaaaatgtaaggaagccatggaaataaaaacgtaggtgtgccttctgtaggctgctacgctcctgtgcacgagggcgtctagaactttgccctccatgcacaagttgcagagcaccctcatcaggacatttacgaaggccctggggtgggatgggcactgcctatgtggccctcccccagcccagcagtatgcagtggcccgggtccaatcaaaggtcgcctgggagggtgagttgcaagaatctggggaaaagagcccaaggtggctgccgcctgctaacagcttgtctagacaggccccatggggcttcaccgcacattgcgagagctctggccagccccctgcccacttgcaaaagaggctgttggcagcaacacttcaccactagaaacctttactccaattcgaaacatgccttaacgcacagtgtgaattacccactctcgtggcccacagaggttgactcattcaggcccccttttgttcagatgaggaaactgaggctgactccgaagcctgggggctttcagatgtggagtgggtccctgtgcccaggtgatgaggggaccaggcgggtctggagcagggctggagtggggctcagatgtagtaggctggcagttaaaggtgccagatgtgagccaggctgctgggtttgaatcctggagctgcctcatagcagcagtaggactttgggtaacttacataggtgctgtatgcctcagtgacctcatctgtaatatagagatgataagagtacctgtctcattggtctactgagttgtccggattaactcattaaatgagttaaaactcatgaagcccttggaactgtgactgacacatagtaagtactcaataaaaaataactgctaagaccagccacagtggctcacacctgtaatctgagcattctgggaggccaaggcggaagaatcccttgagcccagtatttcaagaccagcctaaaggtcaacataggcagactctgtctctactatacatttttagattaaatttttataataataataaccactaaaatgtgattactaaagacagcttcttcacagtacaaagagatgctcttctgagtaccaactctttggaggataaactgcccttataccttcaaaaataacacttgccatatatcaagtcctttcaagtacctggagatttacccagcactctgagataaataccattatccctctgggcacacagaggctcagagaggtttagtcatttgcccaaagtcacacagcctgtacgaggccaggctgggactcaaactcagttctgactgattctaaaatcatgtgtttaactgctgcactctaggaccacccgcaatggatctgtgaaccagaaccagctctggttctgacctgcctagtagggcctttggcatttgggggaggaggccattggaagtccgaagcccccttccagattaggcatgattgcagtaagagaagagacagaccctttggccccccacccctgctcaggctcaaaaatgcagaccctgccgaaacagtccttctcacccagaagcaccccatagggtgggctgagtaaccttgggggcctcgtcagtcttgggctgccccatgccctgcacagcccgcctgaggtttgaggaaggggcagttggctaggcccagactggagaaagccaccccaccatggctcttctgcaagaacccccggccagccacaagcctaagccccctccttaaaagctcctcctctgaccttagctgtgcatcaagggagaaaagaaagctccaggccgggtgcggtggctcacacctgcaatcccagcactttgggagaccaaggctggcagatcattaggtcaggagttcgagaccagcctggccagcaaggtgaaaccccatctctactaaaattacaaaaaattagtcaggcatggtgacacgtgcctgtagtcccagctactctggaggctgaggcaggagaattgcttgaacccaggaggcgaaggttgcagtaaaccaagatcacgccactacactccagcctgggcgacagagcaagactctgtctc

SEQ ID NO: 15 - DNA, 779 bp, Homo sapiens
  misc_feature 1..779: note = lncFIB #1: ENSG00000227811
  source 1..779: mol_type = other DNA
gggaaggtggctcccgcctcactcctagagtgaaatcctcgcagccctgccctcgcgggcggagcagagacgccgccgcttggccggttcacgcctgtcacacaaagccgccgagggtcggctgcgcccctggcaaggctgtagggctggggatactgagtatgacaggcgaggtccctgctctcaagaacttacatgggagagaaggggagacaagataaacaggagccagagaattctcgtagctgtgattagacatcaaccttgggcatgctaaacatttcagcctggacaccctgagaagagccagtgccactggtgacagctgcttctgtgcgggcttttgcttggccagagaggcaggaccttctccctggcattttgaggagtggtgccgggtgggcagtgactgcactagcaacagtctataccaagagaagacctcaaattcccagagaagaaaggcagcgaggccaagaaatgctgccgccgcttaacaagtgaagatgcatactggagaataggaactgaacacgcgaaggagtttcggctttactgccttctctcctagcattgaacagatcttctctgctgttgattattctccatgaatcatcatccatagaaagtgactgaggcaatagagagtgaataatgaatctctcaaatagtcttttgtgtttctgcctggagggttttggaaataacttgcatcttaatctttctgacttagaatcatagaatttgggagctgaaaaggaccttcatcagggcatgtg

SEQ ID NO: 16 - DNA, 1351 bp, Homo sapiens
  misc_feature 1..1351: note = lncFIB #2: ENSG00000253819
  source 1..1351: mol_type = other DNA
tgcactcaccagataaaagtccaatgcactctgtggggtgaaacagcctctgtctcaggttgggagttgctgccatggagaccacaagagtggagattttctgacacacagaaaggaatggattacagctccttcttatgtaaatttccatggaggggaaaaaagattaaaaggaaaataattctgctttagtcgttttcctttaaggtgataagatcctttgattccatcgtggagaagaaggcaacagctgtttgtcacaaatgatgggaaatgtgccaacgtcacaacaaggtttgagggaggcacatctcatgctcggacatgaacacccgatcatcatgcttatgaactacaaaaggatctccagtatgggctcacggcaggtaattgttttgagacttatgtctgaaatgtttgtatcccacccaaatatttaattgttaactttgataggtgtagaattctaggctagaaataatttttttctacagaattctaaagtcattgttacattatctttttgcttctactggggctgttgagaaattcacagtcattttggtattttgagcaatgatttttacctttttctgaaaagttttaaaattccctcttttatgtctattgttctgaaagtttaagatgatggaacttagtgtgagtttatttttatctattgtactgaatatttactgtaccttttaagtctagaaactcatgcccttcagtactgataaatttcttggattatttcttgtgtcttttttcttctatttttcaagttctttttctgaaattttagttactcagttgatgaactcctagaatgattttctatatttcttatcttttatatcatatcttccatctttttgatttttcattctactttctaagagatttccctaaatttatcttctaactatttgactgaaattttcattctggctgtcaaatttgaatttataattactatcttgttttacaattgttttccttttgccagaatattgtttttatttaataaattacaataacttctcctatttgtctgaggatgttgatgatagtttccttttgaagtttttattttcctgcagagttttctataagttgcttttttttcccccataatattttggcctttacagttggcccttgaataacatgaatttgaactgcctgagtctgctaatatgtggattatcttctgcttctgcaccctgagacagcaagaccaacccttcctcttgcttcttttccttagcctattcaacgtgaagatgactaaggtaaggacctttgtgacgatccacttccactcaataaaaaatacatatttttttcc

SEQ ID NO: 17 - DNA, 691 bp, Homo sapiens
  misc_feature 1..691: note = lncFIB #3: ENSG00000250056
  source 1..691: mol_type = other DNA
ccaccgacgagacggactcaggaggggcgaacgctcctgggaaatccagaaactctagagaagcttgtttgccaaaccttgctttgggcggtggagacagaatatcatgtcccagatcagcgtgtggtgactgtggaggcatttacacacagcacactccagctctgccactgggccggctgaggtggctgcggtacacggcgcccatcctgagccccaggttcctcacatgtcaacagcatgaaaagtgcctctaagacatccacaggtgtgcagtgttaaagcaggattctgccatgattgccacctctggagacgtcacagccagcacagccatgaccagggatgaacttggccattgtccagcagcaccatgggaggaccagctgctgtaagtgaaggtcatcatccatctgatttgctggctttttgcaactactgcggtggaagtcttagagtagccaccttctctgtactcagaccttcacttcgccagatgtcagcacagcagtgtcagcagcttcgtcaaacacaaacaccaccacctctctggatcagaagcacatggaaagcccaccttgcaagaagacccagctattccaaaagtacccatatccctgtagcatcctcttagctgaatgtctctactccttaaacttgtaccactttgaagagtaaaaatttaaattca

SEQ ID NO: 18 - DNA, 16 bp, synthetic construct
  misc_feature 1..16: note = Gapmer-lncRNA 1.1
  source 1..16: mol_type = other DNA
aattaccgtgcttacc

SEQ ID NO: 19 - DNA, 16 bp, synthetic construct
  misc_feature 1..16: note = Gapmer-lncRNA 1.2
  source 1..16: mol_type = other DNA
gaaactccttcgcgtg

SEQ ID NO: 20 - DNA, 16 bp, synthetic construct
  misc_feature 1..16: note = Gapmer-lncRNA 4.1
  source 1..16: mol_type = other DNA
gctaatctatgagtga

SEQ ID NO: 21 - DNA, 15 bp, synthetic construct
  misc_feature 1..15: note = Gapmer-lncRNA 4.3
  source 1..15: mol_type = other DNA
ccagaatggcagtaa

SEQ ID NO: 22 - DNA, 16 bp, synthetic construct
  misc_feature 1..16: note = Gapmer-lncRNA 7.1
  source 1..16: mol_type = other DNA
tagtaactctgtagga

SEQ ID NO: 23 - DNA, 16 bp, synthetic construct
  misc_feature 1..16: note = Gapmer-lncRNA 7.2
  source 1..16: mol_type = other DNA
acgcagcagataaaat

SEQ ID NO: 24 - DNA, 16 bp, synthetic construct
  misc_feature 1..16: note = Gapmer-lncRNA 7.3
  source 1..16: mol_type = other DNA
gtagtaactctgtagg

SEQ ID NO: 25 - DNA, 16 bp, synthetic construct
  misc_feature 1..16: note = Gapmer-lncRNA 7.4
  source 1..16: mol_type = other DNA
acagaggtgtcgatta

SEQ ID NO: 26 - DNA, 16 bp, synthetic construct
  misc_feature 1..16: note = Gapmer-lncRNA 8.1
  source 1..16: mol_type = other DNA
tacgctgagtggatga

SEQ ID NO: 27 - DNA, 15 bp, synthetic construct
  misc_feature 1..15: note = Gapmer-lncRNA 8.2
  source 1..15: mol_type = other DNA
ttagcgaatgattaa

SEQ ID NO: 28 - DNA, 16 bp, synthetic construct
  misc_feature 1..16: note = Gapmer-lncRNA 10.1
  source 1..16: mol_type = other DNA
gactcggctaagataa

SEQ ID NO: 29 - DNA, 16 bp, synthetic construct
  misc_feature 1..16: note = Gapmer-lncRNA 10.2
  source 1..16: mol_type = other DNA
gaaatgccgttagagt

SEQ ID NO: 30 - DNA, 16 bp, synthetic construct
  misc_feature 1..16: note = Gapmer-lncRNA 11.1
  source 1..16: mol_type = other DNA
cgtgtggatcaatgag

SEQ ID NO: 31 - DNA, 16 bp, synthetic construct
  misc_feature 1..16: note = Gapmer-lncRNA 11.2
  source 1..16: mol_type = other DNA
tgagtagcatgcaatc

SEQ ID NO: 32 - DNA, 16 bp, synthetic construct
  misc_feature 1..16: note = Gapmer-lncRNA 13.1
  source 1..16: mol_type = other DNA
cagtaacgtcttctta

SEQ ID NO: 33 - DNA, 16 bp, synthetic construct
  misc_feature 1..16: note = Gapmer-lncRNA 13.2
  source 1..16: mol_type = other DNA
acttgaagcctgacta

SEQ ID NO: 34 - DNA, 20 bp, synthetic construct
  misc_feature 1..20: note = IncRNA #1 forward primer
  source 1..20: mol_type = other DNA
gggagagaaggggagacaag

SEQ ID NO: 35 - DNA, 20 bp, synthetic construct
  misc_feature 1..20: note = IncRNA #1 reverse primer
  source 1..20: mol_type = other DNA
ctcaaaatgccagggagaag

SEQ ID NO: 36 - DNA, 20 bp, synthetic construct
  misc_feature 1..20: note = IncRNA #1 forward primer
  source 1..20: mol_type = other DNA
tgatgaaagccactgaggaa

SEQ ID NO: 37 - DNA, 20 bp, synthetic construct
  misc_feature 1..20: note = IncRNA #2 forward primer
  source 1..20: mol_type = other DNA
acatgaacacccgatcatca

SEQ ID NO: 38 - DNA, 20 bp, synthetic construct
  misc_feature 1..20: note = IncRNA #2 reverse primer
  source 1..20: mol_type = other DNA
aagcaagaggaagggttggt

SEQ ID NO: 39 - DNA, 20 bp, synthetic construct
  misc_feature 1..20: note = IncRNA #3 forward primer
  source 1..20: mol_type = other DNA
aaatgaggctcctgggaaat

SEQ ID NO: 40 - DNA, 20 bp, synthetic construct
  misc_feature 1..20: note = IncRNA #3 reverse primer
  source 1..20: mol_type = other DNA
tgcacacctgtggatgtctt

SEQ ID NO: 41 - DNA, 20 bp, synthetic construct
  misc_feature 1..20: note = IncRNA #4 forward primer
  source 1..20: mol_type = other DNA
cttctaaaggctgcctgcat

SEQ ID NO: 42 - DNA, 21 bp, synthetic construct
  misc_feature 1..21: note = IncRNA #4 reverse primer
  source 1..21: mol_type = other DNA
gaccttggattatgaggtgga

SEQ ID NO: 43 - DNA, 20 bp, synthetic construct
  misc_feature 1..20: note = IncRNA #5 forward primer
  source 1..20: mol_type = other DNA
actctccgagggtcaaggat

SEQ ID NO: 44 - DNA, 20 bp, synthetic construct
  misc_feature 1..20: note = IncRNA #5 reverse primer
  source 1..20: mol_type = other DNA
cacccatcaaaggatggaat

SEQ ID NO: 45 - DNA, 20 bp, synthetic construct
  misc_feature 1..20: note = IncRNA #6 forward primer
  source 1..20: mol_type = other DNA
acagactgcgcccttgtact

SEQ ID NO: 46 - DNA, 19 bp, synthetic construct
  misc_feature 1..19: note = IncRNA #6 reverse primer
  source 1..19: mol_type = other DNA
acgtgaggctgcataatgg

SEQ ID NO: 47 - DNA, 20 bp, synthetic construct
  misc_feature 1..20: note = IncRNA #7 forward primer
  source 1..20: mol_type = other DNA
catctgcactggtgtgaggt

SEQ ID NO: 48 - DNA, 20 bp, synthetic construct
  misc_feature 1..20: note = IncRNA #7 reverse primer
  source 1..20: mol_type = other DNA
tatccaagtgggagctgctt

SEQ ID NO: 49 - DNA, 20 bp, synthetic construct
  misc_feature 1..20: note = IncRNA #7 forward primer
  source 1..20: mol_type = other DNA
cttctgggtcctcttgcttg

SEQ ID NO: 50 - DNA, 20 bp, synthetic construct
  misc_feature 1..20: note = IncRNA #7 reverse primer
  source 1..20: mol_type = other DNA
cagggagcacacaatctcct

SEQ ID NO: 51 - DNA, 20 bp, synthetic construct
  misc_feature 1..20: note = IncRNA #7 reverse primer
  source 1..20: mol_type = other DNA
gatggagactggccttggta

SEQ ID NO: 52 - DNA, 20 bp, synthetic construct
  misc_feature 1..20: note = IncRNA #8 forward primer
  source 1..20: mol_type = other DNA
ccagaaagaggctgcaaagt

SEQ ID NO: 53 - DNA, 20 bp, synthetic construct
  misc_feature 1..20: note = IncRNA #8 reverse primer
  source 1..20: mol_type = other DNA
cagttcctcgatggcatctt

SEQ ID NO: 54 - DNA, 20 bp, synthetic construct
  misc_feature 1..20: note = IncRNA #9 forward primer
  source 1..20: mol_type = other DNA
ggagagaaggaaagggagga

SEQ ID NO: 55 - DNA, 20 bp, synthetic construct
  misc_feature 1..20: note = IncRNA #9 reverse primer
  source 1..20: mol_type = other DNA
gggctacaattgacctccag

SEQ ID NO: 56 - DNA, 20 bp, synthetic construct
  misc_feature 1..20: note = IncRNA #10 forward primer
  source 1..20: mol_type = other DNA
tgcgtctcttcttgcagcta

SEQ ID NO: 57 - DNA, 20 bp, synthetic construct
  misc_feature 1..20: note = IncRNA #10 reverse primer
  source 1..20: mol_type = other DNA
gcagtcccaactcagctttc

SEQ ID NO: 58 - DNA, 20 bp, synthetic construct
  misc_feature 1..20: note = IncRNA #10 forward primer
  source 1..20: mol_type = other DNA
gctccttgcaagtcaccttc

SEQ ID NO: 59 - DNA, 20 bp, synthetic construct
  misc_feature 1..20: note = IncRNA #10 reverse primer
  source 1..20: mol_type = other DNA
ttacaaagcagcctgtgtgc

SEQ ID NO: 60 - DNA, 22 bp, synthetic construct
  misc_feature 1..22: note = IncRNA #11 forward primer
  source 1..22: mol_type = other DNA
attagaaaactgaggcccagag

SEQ ID NO: 61 - DNA, 26 bp, synthetic construct
  misc_feature 1..26: note = IncRNA #11 reverse primer
  source 1..26: mol_type = other DNA
cagtattttctttcactaggggtcaa

SEQ ID NO: 62 - DNA, 20 bp, synthetic construct
  misc_feature 1..20: note = IncRNA #11 forward primer
  source 1..20: mol_type = other DNA
tagctgccagaggtcacaca

SEQ ID NO: 63 - DNA, 20 bp, synthetic construct
  misc_feature 1..20: note = IncRNA #11 reverse primer
  source 1..20: mol_type = other DNA
atctagccgcttcactccaa

SEQ ID NO: 64 - DNA, 19 bp, synthetic construct
  misc_feature 1..19: note = IncRNA #12 forward primer
  source 1..19: mol_type = other DNA
ccctacccagagaccctga

SEQ ID NO: 65 - DNA, 20 bp, synthetic construct
  misc_feature 1..20: note = IncRNA #12 reverse primer
  source 1..20: mol_type = other DNA
cagctacttgggaggctgag

SEQ ID NO: 66 - DNA, 20 bp, synthetic construct
  misc_feature 1..20: note = IncRNA #12 reverse primer
  source 1..20: mol_type = other DNA
tgcaagagtattcggctggt

SEQ ID NO: 67 - DNA, 18 bp, synthetic construct
  misc_feature 1..18: note = IncRNA #12 forward primer
  source 1..18: mol_type = other DNA
ctccgggccctgatagat

SEQ ID NO: 68 - DNA, 20 bp, synthetic construct
  misc_feature 1..20: note = IncRNA #12 reverse primer
  source 1..20: mol_type = other DNA
attttgagtgagggctggaa

SEQ ID NO: 69 - DNA, 20 bp, synthetic construct
  misc_feature 1..20: note = IncRNA #13 forward primer
  source 1..20: mol_type = other DNA
tttcatccaagcctggaaat

SEQ ID NO: 70 - DNA, 20 bp, synthetic construct
  misc_feature 1..20: note = IncRNA #13 reverse primer
  source 1..20: mol_type = other DNA
ggagcctgagtttcgaagtg

SEQ ID NO: 71 - DNA, 20 bp, synthetic construct
  misc_feature 1..20: note = IncRNA #13 reverse primer
  source 1..20: mol_type = other DNA
agtccctgaggttggtaggg

SEQ ID NO: 72 - DNA, 20 bp, synthetic construct
  misc_feature 1..20: note = IncRNA #14 forward primer
  source 1..20: mol_type = other DNA
atggcaggcttcagagagaa

SEQ ID NO: 73 - DNA, 20 bp, synthetic construct
  misc_feature 1..20: note = IncRNA #14 reverse primer
  source 1..20: mol_type = other DNA
gggactccggtatagcatca

SEQ ID NO: 74 - DNA, 19 bp, synthetic construct
  misc_feature 1..19: note = IncRNA #15 forward primer
  source 1..19: mol_type = other DNA
ccatgctgatgtcagagaa

SEQ ID NO: 75 - DNA, 20 bp, synthetic construct
  misc_feature 1..20: note = IncRNA #15 reverse primer
  source 1..20: mol_type = other DNA
cctctgtgttccccatgtct

SEQ ID NO: 76 - DNA, 20 bp, synthetic construct
  misc_feature 1..20: note = IncRNA #15 forward primer
  source 1..20: mol_type = other DNA
tcaaactcctgggctcaagt

SEQ ID NO: 77 - DNA, 20 bp, synthetic construct
  misc_feature 1..20: note = IncRNA #15 reverse primer
  source 1..20: mol_type = other DNA
gctcagctgggaatcagaac

SEQ ID NO: 78 - DNA, 23 bp, synthetic construct
  misc_feature 1..23: note = IncRNA #15 forward primer
  source 1..23: mol_type = other DNA
cagaggctgggtctaattagttg

SEQ ID NO: 79 - DNA, 20 bp, synthetic construct
  misc_feature 1..20: note = IncRNA #15 reverse primer
  source 1..20: mol_type = other DNA
ggaggctgcttctccagagt

(19) United States
(12) Patent Application Publication
(10) Pub. No.: US 2026/0117226 A1
(43) Pub. Date: Apr. 30, 2026

(54) LONG NON-CODING RNAS AS TARGET FOR TREATING FIBROSIS AND CANCER

(71) Applicant: MEDIZINISCHE HOCHSCHULE HANNOVER, Hannover (DE)

(72) Inventors: Thomas THUM, Hannover (DE); Sonja GROSS, Hannover (DE); Christian BÄR, Hannover (DE)

(21) Appl. No.: 18/877,651

(22) PCT Filed: Jun. 29, 2023
(86) PCT No.: PCT/EP2023/067753

§ 371 Date: Dec. 20, 2024

(30) Foreign Application Priority Data
Jun. 30, 2022 (EP) 22182222.4

(51) Int. Cl. C12N 15/113; A61P 1/16; A61P 9/00; A61P 11/00; A61P 35/00; C12Q 1/6883; C12Q 1/6886

(52) U.S. Cl. CPC C12N 15/113; A61P 1/16; A61P 9/00; A61P 11/00; A61P 35/00; C12Q 1/6883; C12Q 1/6886; C12N 2310/11; C12N 2310/141; C12Q 2600/158

(57) ABSTRACT

The present invention relates to a pharmaceutical composition comprising (i) a compound inhibiting the expression and/or the activity of one or more lncRNAs selected from SEQ ID NOs 1 to 14; and/or (ii) a compound promoting the expression and/or the activity of one or more long non-coding RNAs (lncRNAs) selected from SEQ ID NOs 15 to 17.

### RELATED PATENT APPLICATIONS

This patent application is a 35 U.S.C. 371 national phase patent application of PCT/EP2023/067753 filed on Jun. 29, 2023, entitled “LONG NON-CODING RNAS AS TARGET FOR TREATING FIBROSIS AND CANCER”, naming Thomas THUM et al. as inventors, and designated by attorney docket no. AF1955 PCT, which claims priority to European Application No. 22182222.4 filed on Jun. 30, 2022, entitled “LONG NON-CODING RNAS AS TARGET FOR TREATING FIBROSIS AND CANCER,” naming Thomas THUM et al. as inventors. The entire content of the foregoing patent applications is incorporated herein by reference, including all text, tables and drawings.

### SEQUENCE LISTING

The instant application contains a Sequence Listing which has been submitted electronically in XML format and is hereby incorporated by reference in its entirety. Said XML copy is named “4_AF1955 PCT Sequence Listing—as filed” and is 140 kilobytes in size.

The present invention relates to a pharmaceutical composition comprising (i) a compound inhibiting the expression and/or the activity of one or more lncRNAs selected from SEQ ID NOs 1 to 14 and/or (ii) a compound promoting the expression and/or the activity of one or more long non-coding RNAs (lncRNAs) selected from SEQ ID NOs 15 to 17.

In this specification, a number of documents including patent applications and manufacturer's manuals are cited. The disclosure of these documents, while not considered relevant for the patentability of this invention, is herewith incorporated by reference in its entirety. More specifically, all referenced documents are incorporated by reference to the same extent as if each individual document was specifically and individually indicated to be incorporated by reference.

In the modern civilization, cardiovascular diseases (CVDs) are gaining increasing importance mainly due to a life style characterized by an unhealthy diet and physical inactivity.^{1,2} The high mortality is an additional factor that highlights the urgency of new therapies.³ So far, the treatment has predominantly been geared towards symptom reduction and decelerating the disease progression without significantly improving the cardiac output. Thus, research intensely focuses on new strategies to reduce pathological cardiac hypertrophy and fibrosis, the two major features of CVDs.

The heart is a vital organ that is able to adapt to certain conditions. However, once the tissue is damaged, the underlying regenerative capacity of the heart in humans as well as in other mammals is quite restricted.⁴ Maladaptive remodeling processes can be initiated, for example, by persistent high blood pressure or heart attacks.^{5,6} This initially leads to an enlargement of the heart muscle to overcome the increased needs.⁷ Since those measures are not sufficient for a long-term compensation, the tissue has to be further stabilized to avoid a ventricular wall rupture. Therefore, the formation of connective tissue is induced by cardiac fibroblasts, which promotes a progressive stiffening of the tissue referred to as fibrosis.^{7,8} As a result, the heart loses more and more of its performance, ultimately leading to heart failure, where the only way to cure is depending on a very limited heart transplant.⁹ The cardiomyocytes as functional units of the heart muscle are focus of most investigations.

Furthermore, 85% of CVD deaths can be attributed to myocardial infarction or strokes,³ implying that the regenerative capacity of the myocardium needs to be improved extensively. However, the pathological remodeling involving hypertrophy, fibrosis and endothelial dysfunction is caused by a complex interplay between the different cell types within the heart.^{7,8,10} With the newly gained knowledge over the past decades, novel therapeutic strategies could be developed e.g. to reduce cardiomyocyte hypertrophy on a molecular level. Our institute has a leading function in this regard, since Cardior Pharmaceuticals has started a successful in-human clinical trial with a microRNA (miRNA) inhibitor two years ago showing promising advances so far.¹¹

In healthy conditions, fibroblasts are important players to maintain the tissue integrity not only by providing a mechanically supportive surrounding, but they are also crucial for the electrical coupling and cardiomyocyte function. They become activated as a response to an injury, or due to chronic pressure or volume overload.^{8,12} Targeted treatments to reduce the developing fibrosis directly are still very rare and rather ineffective in reversing pathological changes.^{13,14} Hence, there is a need to identify suitable therapeutic targets in order to treat or prevent fibrosis.

Moreover, cancer is a leading cause of death worldwide, accounting for nearly 10 million deaths in 2020, or nearly one in six deaths. The most common cancers are breast, lung, colon and rectum and prostate cancers. Fibrosis plays an important role in cancer progression. Cancers are characterized by extracellular matrix (ECM) deposition, remodeling, and cross-linking that drive fibrosis to stiffen the stroma and promote malignancy. The stiffened stroma enhances tumor cell growth, survival and migration and drives a mesenchymal transition. A stiff ECM also induces angiogenesis, hypoxia and compromises anti-tumor immunity. Tumor aggression and poor patient prognosis correlate with degree of tissue fibrosis and level of stromal stiffness (Piersma et al. (2020), Biochim Biophys Acta Rev Cancer;1873(2):188356.).

Hence, there is an ongoing need to identify new therapeutic targets, in particular in order to treat or prevent fibrosis and cancer. This need is addressed by the present application.

The present invention therefore relates in a first aspect to a pharmaceutical composition comprising (i) a compound inhibiting the expression and/or the activity of one or more lncRNAs selected from SEQ ID NOs 1 to 14 and/or (i) a compound promoting the expression and/or the activity of one or more long non-coding RNAs (lncRNAs) selected from SEQ ID NOs 15 to 17.

In other terms the present invention relates in a first aspect to a compound which is (i) a compound inhibiting the expression and/or the activity of one or more lncRNAs selected from SEQ ID NOs 1 to 14; and/or (ii) a compound promoting the expression and/or the activity of one or more long non-coding RNAs (lncRNAs) selected from SEQ ID NOs 15 to 17 for use as a pharmaceutical composition or a medicament.

In accordance with the present invention, the term “pharmaceutical composition” (or “medicament”) relates to a composition for administration to a patient, preferably a human patient. The pharmaceutical composition of the invention comprises the compound recited above. It may, optionally, comprise further molecules capable of altering the characteristics of the compound of the invention thereby, for example, stabilizing, modulating and/or activating their function. The composition may be in solid, liquid or gaseous form and may be, inter alia, in the form of (a) powder(s), (a) tablet(s), (a) solution(s) or (an) aerosol(s).

These pharmaceutical compositions can be administered to the subject at a suitable dose. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The therapeutically effective amount for a given situation will readily be determined by routine experimentation and is within the skills and judgement of the ordinary clinician or physician. Generally, the regimen as a regular administration of the pharmaceutical composition should be in the range of 1 μg to 5 g units per day. However, a more preferred dosage might be in the range of 0.01 mg to 100 mg, even more preferably 0.01 mg to 50 mg and most preferably 0.01 mg to 10 mg per day.

Furthermore, if for example said compound is a nucleic acid sequence, such as an siRNA, the total pharmaceutically effective amount of pharmaceutical composition administered will typically be less than about 75 mg per kg of body weight, such as for example less than about 70, 60, 50, 40, 30, 20, 10, 5, 2, 1, 0.5, 0.1, 0.05, 0.01, 0.005, 0.001, or 0.0005 mg per kg of body weight. More preferably, the amount will be less than 2000 nmol of nucleic acid sequence (e.g., about 4.4×1016 copies) per kg of body weight, such as for example less than 1500, 750, 300, 150, 75, 15, 7.5, 1.5, 0.75, 0.15, 0.075, 0.015, 0.0075, 0.0015, 0.00075 or 0.00015 nmol of siRNA agent per kg of body weight.

The length of treatment needed to observe changes and the interval following treatment for responses to occur vary depending on the desired effect. The particular amounts may be determined by conventional tests which are well known to the person skilled in the art.

Pharmaceutical compositions of the invention preferably comprise a pharmaceutically acceptable carrier or excipient. By “pharmaceutically acceptable carrier or excipient” is meant a non-toxic solid, semisolid or liquid filler, diluent, encapsulating material or formulation auxiliary of any type (see also Handbook of Pharmaceutical Excipients 6ed. 2010, Published by the Pharmaceutical Press). Examples of suitable pharmaceutical carriers and excipients are well known in the art and include phosphate buffered saline solutions, water, emulsions, such as oil/water emulsions, various types of wetting agents, sterile solutions, organic solvents including DMSO etc. Compositions comprising such carriers or excipients can be formulated by well-known conventional methods. The pharmaceutical composition may be administered, for example, orally, parenterally, such as subcutaneously, intravenously, intramuscularly, intraperitoneally, intrathecally, transdermally, transmucosally, subdurally, locally or topically via iontopheresis, sublingually, by inhalation spray, aerosol or rectally and the like in dosage unit formulations optionally comprising conventional pharmaceutically acceptable carriers or excipients.

The term “ncRNA” or “non-coding RNA” as used herein designates a functional RNA molecule that is not translated into a protein. The DNA sequence from which a non-coding RNA is transcribed is often called in the art an RNA gene. The term “lncRNA” or “long non-coding RNA” is commonly used in the art and designates an ncRNA comprising more than 200 nucleotides. SEQ ID NOs 1 to 17 comprise sequences of more than 200 nucleotides and are therefore lncRNAs. It is of note that SEQ ID NOs 1 to 17 in the sequence listing that forms part of the present disclosure are shown as cDNA sequences. The corresponding lncRNA sequences correspond to these cDNA sequences, wherein T is replaced by U. Hence, for example, the lncRNA of SEQ ID NO: 1 expressed in humans designates the sequence of SEQ ID NO: 1 as shown in the sequence listing, wherein T is replaced by U.

The term “nucleic acid sequence” or “nucleotide sequence”, in accordance with the present invention, includes DNA and RNA. As will be further detailed herein below, nucleotide-based compounds inhibiting the expression of an lncRNA of SEQ ID NOs 1 to 14 may comprise DNA sequences (e.g. LNA GapmeRs) or RNA sequences (e.g. siRNAs). As will be also further detailed herein below, nucleotide-based compounds inhibiting the expression of an lncRNA of SEQ ID NOs 1 to 14 may be single stranded (e.g. LNA GapmeRs) or double-stranded (e.g. siRNAs). The term “nucleic acid sequence” is interchangeably used in accordance with the invention with the term “polynucleotide sequence”. Short “nucleic acid sequence” are also referred to herein as “oligonucleotide sequences”. Oligonucleotide sequences are less than 50 nucleotides in length, preferably less than 40 nucleotides and most preferably less than 30 nucleotides.

The compounds of the invention may be formulated as vesicles, such as liposomes.

Liposomes have attracted great interest because of their specificity and the duration of action they offer from the standpoint of drug delivery. Liposomal delivery systems have been used to effectively deliver nucleic acids, such as siRNA in vivo into cells (Zimmermann et al. (2006) Nature, 441:111-114). Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Non-cationic liposomes, although not able to fuse as efficiently with the cell wall, are phagocytosed by macrophages and other cells in vivo.

A compound inhibiting the expression of one or more lncRNAs selected from SEQ ID NOs 1 to 14—as defined herein in item (i)—is in accordance with the present invention a compound lowering or preventing the transcription of one or more of the genes encoding the lncRNAs selected of SEQ ID NOs 1 to 14. Such compounds include compounds interfering with the transcriptional machinery and/or its interaction with the promoter of said genes and/or with expression control elements remote from the promoter such as enhancers. The compound inhibiting the expression of an lncRNA selected from SEQ ID NOs 1 to 14 specifically inhibits the expression of said lncRNA, for example, by specifically interfering with the promoter region controlling the expression of the lncRNA. Preferably, the transcription of an lncRNAs selected from SEQ ID NOs 1 to 14 is reduced by at least 50%, more preferred at least 75% such as at least 90% or 95%, even more preferred at least 98% and most preferred by about 100%. A compound inhibiting the activity of an lncRNAs selected from SEQ ID NOs 1 to 14—as defined herein in item (i)—in accordance with the present invention causes said lncRNA to perform its function with lowered efficiency. The compound inhibiting the activity of an lncRNA selected from SEQ ID NOs 1 to 14 specifically inhibits the activity of said lncRNA. Preferably, the activity of an lncRNA selected from SEQ ID NOs 1 to 14 is reduced by at least 50%, more preferred at least 75% such as at least 90% or 95%, even more preferred at least 98%, and most preferably about 100%. Means and methods for determining the reduction of activity of an RNA are established in the art and are described, for example, in Esau et al. (2004), JBC, 279:52361-52365 or Gribbings et al. (2009), Nature Cell Biology 11, 1143-1149.

Compounds as defined herein in item (i) that inhibits the activity of one or more lncRNAs may be an antisense molecule, siRNA, shRNA, antibody, ribozyme, aptamer, or small molecule. In this connection it is to be understood that, for example, siRNA and shRNA mediate RNA interference (RNAi). RNAi is generally defined as a process in which RNA molecules are involved in sequence-specific suppression of gene expression by double-stranded RNA, through translational or transcriptional repression. Hence, RNAi is a post-transcriptional gene silencing mechanism, as discussed above, lncRNAs are expressed but not translated into protein. lncRNAs exert their biological activity as an RNA molecule. However, RNAi also works with lncRNAs just as for mRNAs because, for example, siRNA and shRNA are also capable of binding to target lncRNA by complementary base pairing, noting that the thereby formed dsRNA then becomes degraded in cells, so that lncRNA no longer can display their biological activity.

The efficiency of an inhibiting compound can be quantified by methods comparing the level of activity in the presence of the inhibitor to that in the absence of the inhibitor. For example, as an activity measure may be used: the change in amount of lncRNA formed. Such a method may be effected in high-throughput format in order to test the efficiency of several inhibiting compound simultaneously. High-throughput assays, independently of being biochemical, cellular or other assays, generally may be performed in wells of microtiter plates, wherein each plate may contain 96, 384 or 1536 wells. Handling of the plates, including incubation at temperatures other than ambient temperature, and bringing into contact of test compounds with the assay mixture is preferably affected by one or more computer-controlled robotic systems including pipetting devices. In case large libraries of test compounds are to be screened and/or screening is to be effected within short time, mixtures of, for example 10, 20, 30, 40, 50 or 100 test compounds may be added to each well. In case a well exhibits the expected activity, said mixture of test compounds may be de-convoluted to identify the one or more test compounds in said mixture giving rise to said activity.

A compound promoting the expression of one or more lncRNAs selected from SEQ ID NOs 15 to 17—as defined herein in item (ii)—may be any compound enhancing or upregulating the transcription of an lncRNA selected from SEQ ID NOs 15 to 17. Non-limiting examples of such compounds are transcription factors enhancing the transcription of the genes encoding the lncRNAs selected from SEQ ID NOs 13 to 15 or a small molecule enhancing the expression of one or more lncRNAs selected from SEQ ID NOs 13 to 15. A transcription factor is a protein binding to specific DNA sequences, thereby controlling the transcription of genetic information from DNA to RNA. A small molecule is a low molecular weight compound which is by definition not a polymer. A compound promoting the activity of one or more lncRNAs selected from SEQ ID NOs 15 to 17—as defined herein in item (i)—may be any compound which causes that said lncRNA effectively performs its function in a cell. Hence, in the simplest form such a compound may be a recombinantly produced or isolated lncRNAs selected from SEQ ID NOs 15 to 17 or any precursor or active/functional fragment thereof. In this embodiment the administration of a recombinantly produced or isolated lncRNA increases the concentration of lncRNA in the subject to be treated. This higher concentration promotes the overall activity of the respective lncRNA in the subject. The fragments have to retain or essentially retain the function of the full-length lncRNA. Such a compound may also be a vector or host being capable of producing such an lncRNAs. Hence, the fragments have to be functional fragments. Also orthologous or homologous sequences of the lncRNA selected from SEQ ID NOs 15 to 17 from different species including precursors or active/functional functional fragments thereof may be used.

Alternatively, such a compound may be a compound maintaining or even enhancing the activity of an lncRNA selected from SEQ ID NOs 15 to 17 by either directly or indirectly interacting with the lncRNA. For instance, such a compound may prevent an lncRNA selected from SEQ ID NOs 15 to 17 from degeneration by RNases or may be an interaction partner, such as another lncRNA, which binds to and promotes the activity of an lncRNA selected from SEQ ID NOs 15 to 17. Compounds as defined herein in item (ii) will be further detailed herein below.

The efficiency of a compound as defined herein in item (ii) can also be quantified by methods comparing the level of expression and/or activity of an lncRNA selected from SEQ ID NOs 15 to 17 in the presence of an expression and/or activity promoting compound of the lncRNA, such as a transcription factor, to that in the absence of said compound. For example, as an activity measure the change in amount of lncRNA formed may be used. The method is preferably effected in high-throughput format as further detailed herein above.

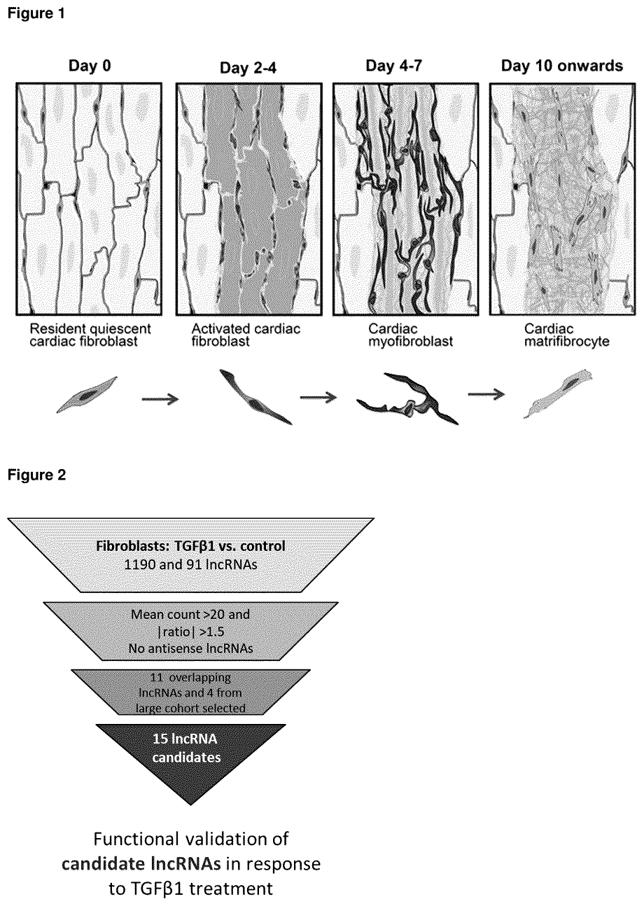

In the examples herein below it is shown that in fibroblasts that have been isolated and treated with TGFβ1 the genes of 15 lncRNAs are significantly deregulated; see FIG. 1. In more detail, the lncRNAs of SEQ ID NOs 1 to 14 (#4-15) are upregulated and the SEQ ID NOs 15 to 17 (#1-3) are downregulated. SEQ ID Nos 1 to 17 as used herein correspond to the 15 lncRNAs #1 to #15 as son in the following Table:

| IncRNA | SEQ ID NOs |
|---|---|
| #1 | 15 |
| #2 | 16 |
| #3 | 17 |
| #4 | 3 |
| #5 | 4 |
| #6 | 5 and 6 |
| #7 | 7 |
| #8 | 8 |
| #9 | 9 |
| #10 | 2 |
| #11 | 1 |
| #12 | 10 |
| #13 | 11 |
| #14 | 12 |
| #15 | 13 and 14 |

The above table shows one SEQ ID NO for lncRNAs #1-5 and 7-14 and two SEQ ID NOs for lncRNAs #6 and 15. This is because for lncRNAs #6 and 15 two splice variants are known.

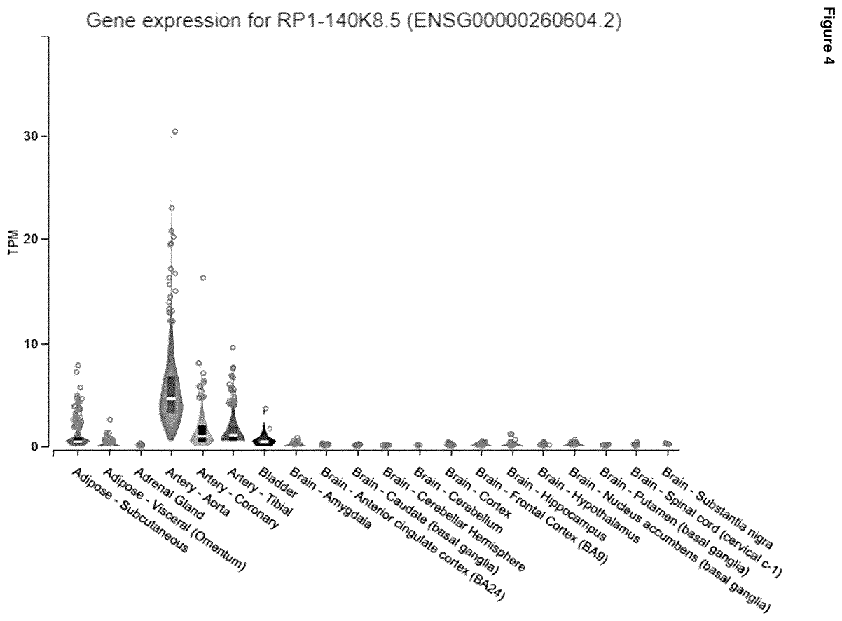

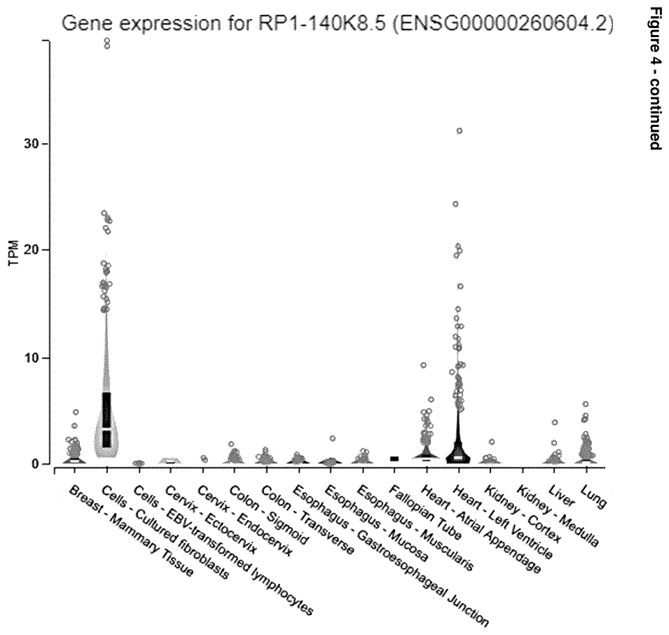

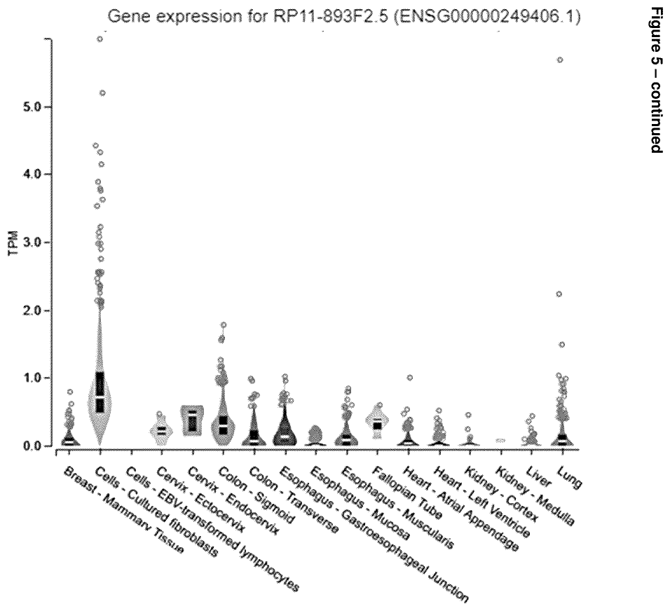

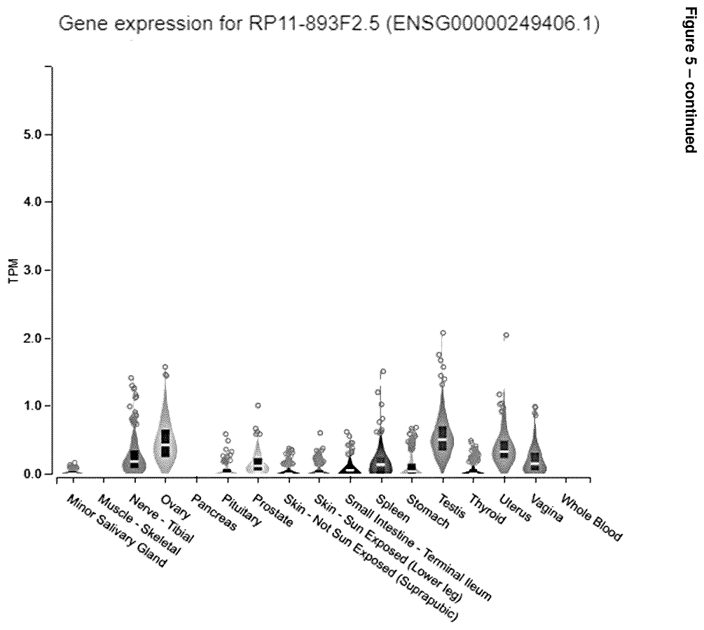

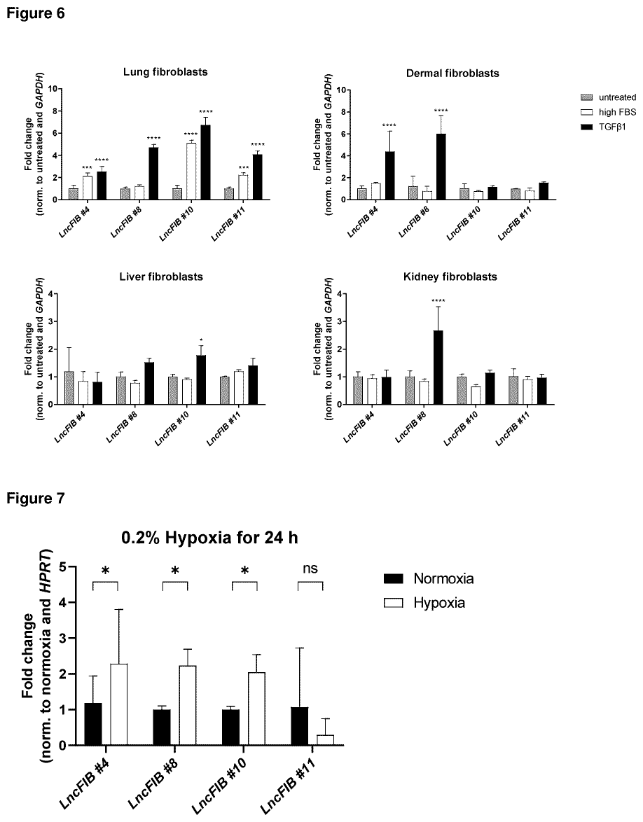

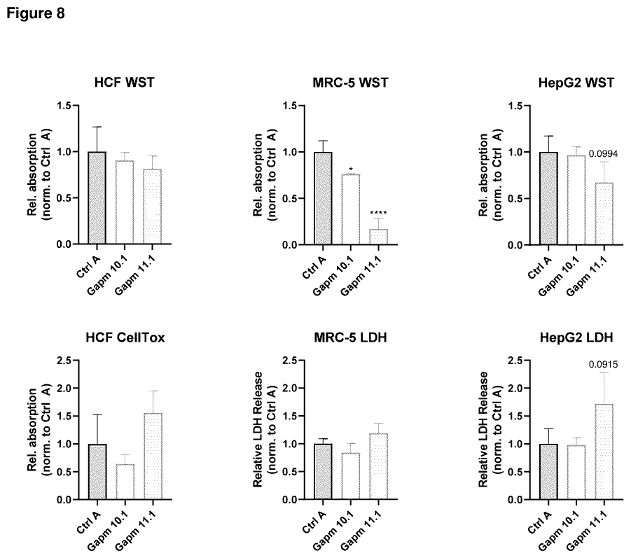

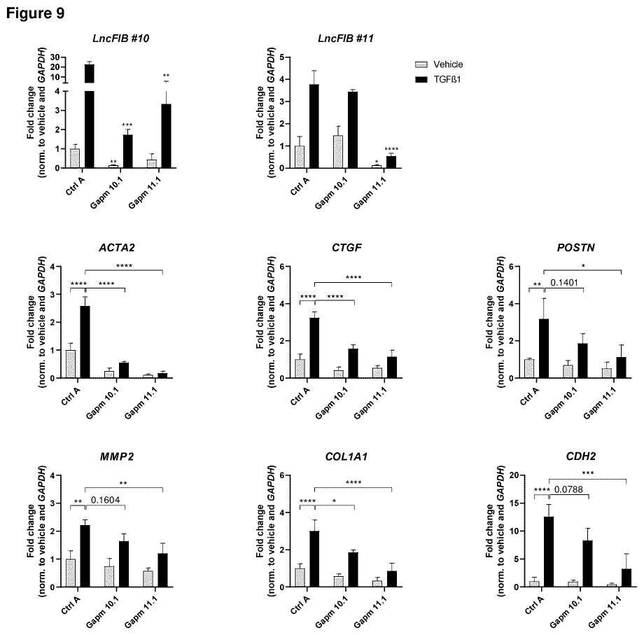

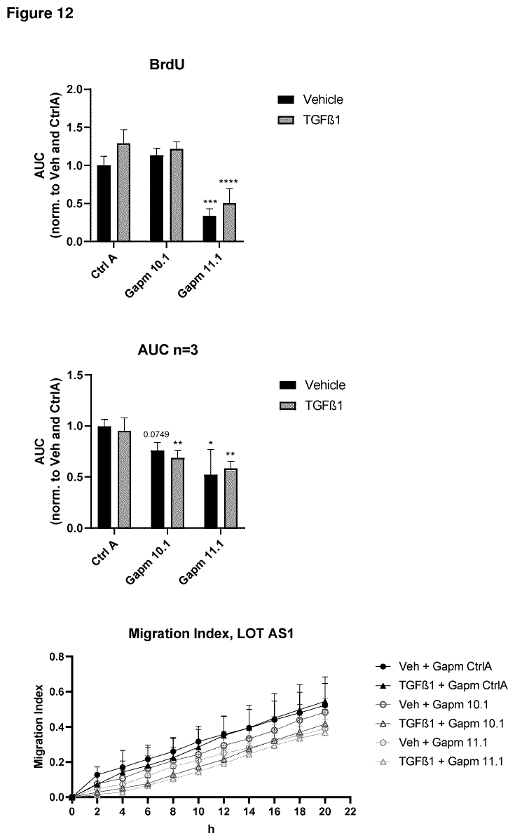

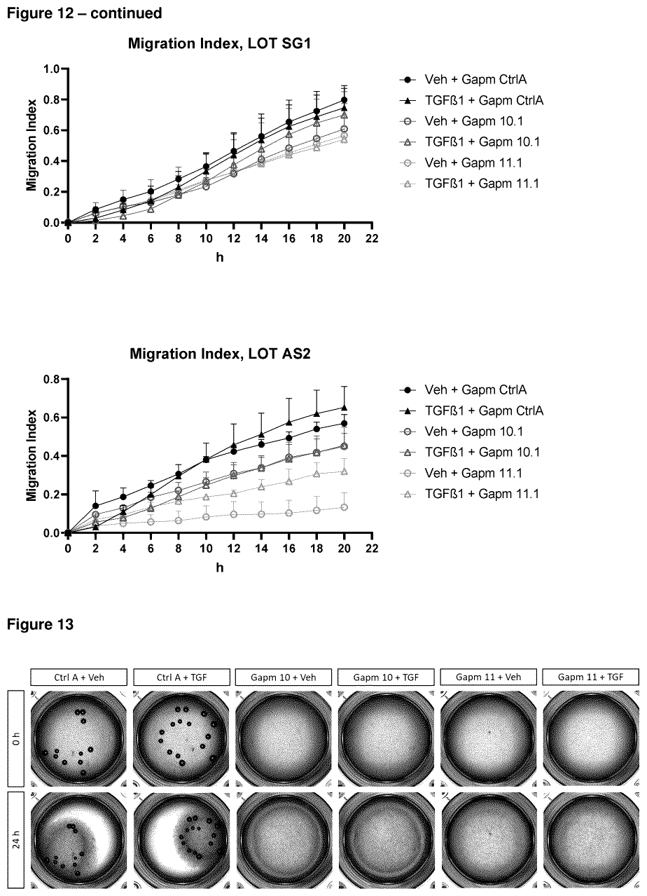

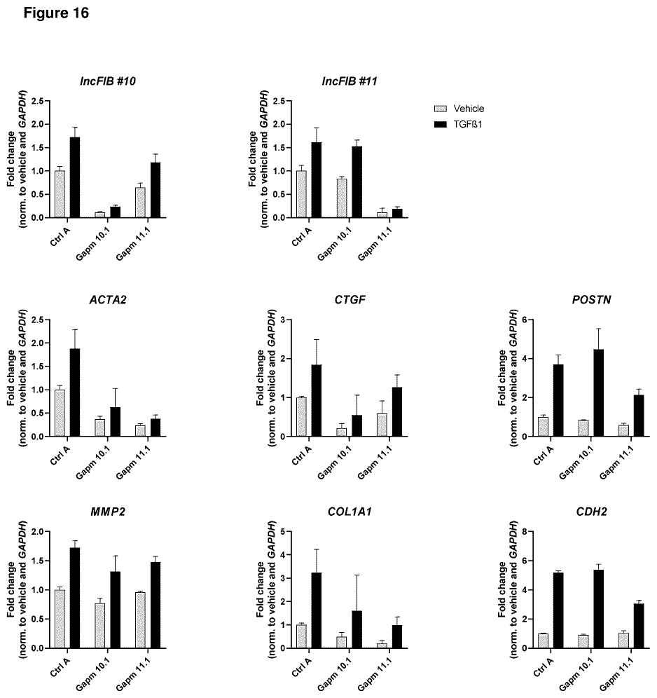

The treatment of isolated fibroblasts with TGFβ1 is a widely used model for fibrosis (see, for example, Chen and Thibeault, Tissue Eng Part A. 2012 December; 18(23-24): 2528-2538; Negmadjanov et al., PLoS One 2015 Apr 7;10(4):e0123046 and Lee et al., Scientific Reports volume 6, Article number: 32231 (2016)). TGFβ1 promotes the proliferation, collagen formation and differentiation of fibroblasts which leads to tissue fibrosis (Kim et al., Cold Spring Harb Perspect Biol. 2018 April; 10(4): a022293.). While deregulation of SEQ ID NOs 1 to 17 has been found in cardiac fibroblasts, FIG. 6 shows that the same can be found in lung, dermal, liver and kidney fibroblasts. That the lncRNAs of SEQ ID NOS 1 to 17 are indeed suitable as targets for therapy is further demonstrated in the examples herein below based on the two lncRNAs of SEQ ID NOs 1 and 2. The GapmeRs #10.1 and #11.1 against these lncRNAs resulted in a strong knockdown of two lncRNAs of SEQ ID NOs 1 and 2 (i.e. LncFIB #11 and #12) in cardiac, lung and liver fibroblasts; see FIG. 9-11. in HCFs. Common fibrosis marker genes associated with the activation of fibroblast, their proliferation, adhesion, migration and the matrix remodeling were significantly downregulated even when stimulated with TGFβ1. These Gapmers also reduced the proliferation and contractile function of cardiac fibroblasts; see FIGS. 12 and 13. The above-discussed data and also the further data in the appended examples evidence that the lncRNAs of SEQ ID NOs 1 to 17 are suitable targets for therapeutic intervention, in particular for the treatment of fibrosis and fibrotic diseases as well as conditions that involve the abnormal proliferation of cells (FIG. 3), such as tumors. Tumors are characterized by extracellular matrix (ECM) deposition, remodeling, and cross-linking that drive fibrosis to stiffen the stroma and promote malignancy. The stiffened stroma enhances tumor cell growth, survival and migration and drives a mesenchymal transition. A stiff ECM also induces angiogenesis, hypoxia and compromises anti-tumor immunity. Tumor aggression and poor patient prognosis correlate with degree of tissue fibrosis and level of stromal stiffness; see, for review, Kim et al., Cold Spring Harb Perspect Biol. 2018 April; 10(4): a022293.). In addition, cancer-associated fibroblasts treated with the GapmeRs #10.1 and #11.1 against the lncRNAs of SEQ ID NOs 1 and 2 showed a reduced metabolic activity (see FIG. 15) and pro-fibrotic TGFβ1 treatment of cancer-associated fibroblasts induced the expression of the lncRNAs of SEQ ID NOs 1 and 2. in addition, a downregulation of fibrosis marker genes even under pro-fibrotic TGFβ1 stimulation was achieved (FIG. 16)For the above reason SEQ ID NOs 1 to 17 are also suitable targets for the treatment of cancer, in particular a fibrotic cancer.

Accordingly, the present invention relates in a second aspect to a compound which is (i) a compound inhibiting the expression and/or the activity of one or more lncRNAs selected from SEQ ID NOs 1 to 14; and/or (ii) a compound promoting the expression and/or the activity of one or more long non-coding RNAs (lncRNAs) selected from SEQ ID NOs 15 to 17 for use in treating or preventing a fibrotic disorder or a tumor, preferably cancer.

The definitions and preferred embodiments of the first aspect of the invention apply mutatis mutandis to the second aspect of the invention, as far as being amenable for combination with the second aspect.

A fibrotic disorder refers to a condition involving fibrosis in one or more tissues. As used herein the term “fibrosis” refers to the formation of fibrous tissue as a reparative or reactive process, rather than as a normal constituent of an organ or tissue. Fibrosis is characterized by fibroblast accumulation and collagen deposition in excess of normal deposition in any particular tissue. As used herein the term “fibrosis” is used synonymously with “fibroblast accumulation and collagen deposition”. Fibroblasts are connective tissue cells, which are dispersed in connective tissue throughout the body. Fibroblasts secrete a nonrigid extracellular matrix containing type I and/or type III collagen. In response to an injury to a tissue, nearby fibroblasts migrate into the wound, proliferate, and produce large amounts of collagenous extracellular matrix. Collagen is a fibrous protein rich in glycine and proline that is a major component of the extracellular matrix and connective tissue, cartilage, and bone. Collagen molecules are triple-stranded helical structures called α-chains, which are wound around each other in a rope-like helix. Collagen exists in several forms or types; of these, type I, the most common, is found in skin, tendon, and bone; and type III is found in skin, blood vessels, and internal organs.

A tumor is an abnormal benign or malignant new growth of tissue that possesses no physiological function and arises from uncontrolled usually rapid cellular proliferation. The tumor is preferably cancer. Cancer is an abnormal malignant new growth of tissue that possesses no physiological function and arises from uncontrolled usually rapid cellular proliferation. The cancer is preferably selected from the group consisting of breast cancer, ovarian cancer, endometrial cancer, vaginal cancer, vulvacancer, bladder cancer, salivary gland cancer, pancreatic cancer, thyroid cancer, kidney cancer, lung cancer, cancer concerning the upper gastrointestinal tract, colon cancer, colorectal cancer, prostate cancer, squamous-cell carcinoma of the head and neck, cervical cancer, glioblastomas, malignant ascites, lymphomas and leukemias. Among this list of cancers gynecologic cancers (i.e. cervical, ovarian, uterine, vaginal, and vulvar cancer) are preferred and breast cancer and ovarian cancer are most preferred. Also preferred among this list of cancers is bladder cancer.

The tumor or cancer is preferably a solid tumor or cancer. A solid tumor or cancer is an abnormal mass of tissue that usually does not contain cysts or liquid areas by contrast to a liquid tumor.

In accordance with a preferred embodiment of the second aspect the fibrotic disorder is selected from systemic sclerosis (SSc), sclerodermatous graft vs. host disease, and nephrogenic systemic fibrosis, as well as numerous organ-specific disorders including radiation-induced fibrosis, organ fibrosis wherein the organ fibrosis is preferably the cardiac fibrosis, pulmonary fibrosis, liver fibrosis, kidney fibrosis, gastrointestinal fibrosis, skeletal muscle fibrosis and multifocal fibrosclerosis.

Among the fibrotic disorders organ fibrosis is preferred and among the organ fibrosis cardiac fibrosis is preferred.

In accordance with another preferred embodiment of the second aspect the cancer is a metastatic cancer or a fibrotic cancer, preferably myelofibrosis.

A metastatic cancer is a cancer that has spread from the part of the body where it started (the primary site) to other parts of the body. When cancer cells break away from a tumor, they can travel to other parts of the body through the bloodstream or the lymph system. The process by which cancer cells spread to other parts of the body is called metastasis.

A fibrotic cancer is cancer wherein a fibrotic response in the tumor microenvironment can be found. A cancer is generally a fibrotic disease (Chandler, Translational Research, Volume 209, July 2019, Pages 55-67). The fibrotic response is termed desmoplasia and is characterized by excessive turnover and remodeling of the extracellular matrix (ECM). Fibrotic cancers, the increased collagen production and crosslinking, as well as altered degradation of the collagenous matrix result in loss of tissue organization and cellular behavior, release of growth factors, and in generation of cryptic sites on collagens with potent signaling activity. All these collagen alterations drive disease progression, immune suppression and affect treatment response. Moreover, these ECM turnover products can be picked up as biomarkers of a specific fibrotic/desmoplastic signature relevant in the clinical setting of oncology.

Non-limiting but preferred examples of fibrotic cancers are myelofibrosis, gastric cancer, pancreatic cancer, Hodgkin's lymphoma, non-Hodgkin's lymphoma, hairy cell leukemia, multiple myeloma, medulloblastoma, myeloid leukemia, acute lymphocytic leukemia, and cancers of the breast, uterus, or colon, including fibroids, fibroma, fibroadenomas and fibrosarcomas.

Among the fibrotic cancers myelofibrosis is preferred. Non-limiting but preferred examples of myelofibrosis are primary myelofibrosis, post-polycythemia vera myelofibrosis, or post-essential thrombocythemia myelofibrosis.

A cancer can also be both, a fibrotic cancer and a metastatic cancer.

In accordance with a preferred embodiment of the first and second aspect of the invention the compound as defined in (i) is a small molecule inhibitor, a nucleotide-based inhibitor or an amino acid-based inhibitor.

A small molecule inhibitor is a low molecular weight organic compound which is by definition not a polymer. The small molecule of the invention is a molecule that binds with high affinity to an lncRNA selected from SEQ ID NOs 1 to 14 and in addition inhibits the activity thereof. The upper molecular weight limit for a small molecule is preferably 1500 Da, more preferably 1000 Da and most preferably 800 Da which allows for the possibility to rapidly diffuse across cell membranes so that they can reach intracellular sites of action. Libraries of small organic molecules and high-throughput techniques for screening such libraries with a specific target molecule, in the present case an lncRNA selected from SEQ ID NOs 1 to 14.

A nucleotide-based inhibitor comprises or consists of a nucleic acid molecule. The sequence of the nucleic acid molecule is preferably complementary to a nucleic acid sequence of at least 12 contiguous nucleotides of lncRNA selected from SEQ ID NOs 1 to 14. The nucleotide-based inhibitor may comprise or consist of RNA, DNA or both. The nucleotide-based inhibitor of the invention is a molecule that binds specifically to an lncRNA selected from SEQ ID NOs 1 to 14 and in addition inhibits the activity thereof. As used herein specific binding means that the inhibitor specifically targets the lncRNA and does substantially not exert any off target inhibitory effects, in particular on other cellular nucleic acid molecules.

An amino acid-based inhibitor comprises or consists of an amino acid sequence and preferably an amino acid sequence of at least 25, more preferably at least 50 amino acids. The amino acid-based inhibitor of the invention is a molecule that binds specifically to an lncRNA selected from SEQ ID NOs 1 to 14 and in addition inhibits its activity. The amino acid-based inhibitor preferably comprises natural amino acids but may also comprise unnatural amino acids. The amino acid-based inhibitor is preferably selected or designed such that it specifically binds to a nucleic acid sequence selected from SEQ ID NOs 1 to 14.

In accordance with a more preferred embodiment of the first and second aspect the nucleotide-based inhibitor is an aptamer, a siRNA, a shRNA, a miRNA, a ribozyme, an antisense nucleic acid molecule, a CRISPR-Cas-based construct, a meganuclease, a zinc finger nuclease, or a transcription activator-like (TAL) effector (TALE) nuclease, and/or the amino acid-based inhibitor is an aptamer, an antibody or an antibody mimetic, wherein the antibody mimetic is preferably selected from affibodies, adnectins, anticalins, DARPins, avimers, nanofitins, affilins, Kunitz domain peptides, Fynomers®, trispecific binding molecules and probodies.

Aptamers in the art have been selected which bind nucleic acid, proteins, small organic compounds, and even entire organisms. A database of aptamers is maintained at http://aptamer.icmb.utexas.edu/. More specifically, aptamers can be classified as DNA or RNA aptamers or peptide aptamers. Whereas the former consist of (usually short) strands of oligonucleotides, the latter consist of a short variable peptide domain, attached at both ends to a protein scaffold. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, and even cells, tissues and organisms. The molecular target envisaged by the present invention is a nucleic acid, namely an lncRNA selected from SEQ ID NOs 1 to 14. Hence, aptamers can be produced against the target molecule of the invention. Aptamers offer the utility for biotechnological and therapeutic applications as they offer molecular recognition properties that rival those of the commonly used biomolecules, in particular antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. Non-modified aptamers are cleared rapidly from the bloodstream, with a half-life of minutes to hours, mainly due to nuclease degradation and clearance from the body by the kidneys, a result of the aptamer's inherently low molecular weight. The rapid clearance of aptamers can be an advantage in applications such as in vivo diagnostic imaging. Several modifications, such as 2′-fluorine-substituted pyrimidines, polyethylene glycol (PEG) linkage, etc. are available with which the half-life of aptamers easily can be increased to the day or even week time scale.

In accordance with the present invention, the term “small interfering RNA (siRNA)”, also known as short interfering RNA or silencing RNA, refers to a class of 18 to 30, preferably 19 to 25, most preferred 21 to 23 or even more preferably 21 nucleotide-long double-stranded RNA molecules that play a variety of roles in biology. Most notably, siRNA is involved in the RNA interference (RNAi) pathway where the siRNA interferes with the expression of a specific gene.

In addition to their role in the RNAi pathway, siRNAs also act in RNAi-related pathways, e.g. as an antiviral mechanism or in shaping the chromatin structure of a genome. siRNAs naturally found in nature have a well-defined structure: a short double-strand of RNA (dsRNA) with 2-nt 3′ overhangs on either end. Each strand has a 5′ phosphate group and a 3′ hydroxyl (—OH) group. This structure is the result of processing by dicer, an enzyme that converts either long dsRNAs or small hairpin RNAs into siRNAs. siRNAs can also be exogenously (artificially) introduced into cells to bring about the specific knockdown of a gene of interest. Essentially any gene for which the sequence is known can thus be targeted based on sequence complementarity with an appropriately tailored siRNA. The double-stranded RNA molecule or a metabolic processing product thereof is capable of mediating target-specific nucleic acid modifications, particularly RNA interference and/or DNA methylation.

Exogenously introduced siRNAs may be devoid of overhangs at their 3′ and 5′ ends, however, it is preferred that at least one RNA strand has a 5′- and/or 3′-overhang. Preferably, one end of the double-strand has a 3′-overhang from 1 to 5 nucleotides, more preferably from 1 to 3 nucleotides and most preferably 2 nucleotides. The other end may be blunt-ended or has up to 6 nucleotides 3′-overhang. In general, any RNA molecule suitable to act as siRNA is envisioned in the present invention. The most efficient silencing was so far obtained with siRNA duplexes composed of 21-nt sense and 21-nt antisense strands, paired in a manner to have a 2-nt 3′-overhang. The sequence of the 2-nt 3′ overhang makes a small contribution to the specificity of target recognition restricted to the unpaired nucleotide adjacent to the first base pair (Elbashir et al. 2001, Nature; 411(6836):494-8). 2′-deoxynucleotides in the 3′ overhangs are as efficient as ribonucleotides, but are often cheaper to synthesize and probably more nuclease resistant. Delivery of siRNA may be accomplished using any of the methods known in the art, for example by combining the siRNA with saline and administering the combination intravenously or intranasally or by formulating siRNA in glucose (such as for example 5% glucose) or cationic lipids and polymers can be used for siRNA delivery in vivo through systemic routes either intravenously (IV) or intraperitoneally (IP) (Fougerolles et al. (2008), Current Opinion in Pharmacology, 8:280-285; Lu et al. (2008), Methods in Molecular Biology, vol. 437: Drug Delivery Systems—Chapter 3: Delivering Small Interfering RNA for Novel Therapeutics).

A short hairpin RNA (shRNA) is a sequence of RNA that makes a tight hairpin turn that can be used to silence gene expression via RNA interference. shRNA uses a vector introduced into cells and utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs which match the siRNA that is bound to it. si/shRNAs to be used in the present invention are preferably chemically synthesized using appropriately protected ribonucleoside phosphoramidites and a conventional DNA/RNA synthesizer. Suppliers of RNA synthesis reagents are Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK). Most conveniently, siRNAs or shRNAs are obtained from commercial RNA oligo synthesis suppliers, which sell RNA-synthesis products of different quality and costs. In general, the RNAs applicable in the present invention are conventionally synthesized and are readily provided in a quality suitable for RNAi.

Further molecules effecting RNAi include, for example, microRNAs (miRNA). Said RNA species are single-stranded RNA molecules. Endogenously present miRNA molecules regulate gene expression by binding to a complementary mRNA transcript and triggering of the degradation of said mRNA transcript through a process similar to RNA interference. Accordingly, exogenous miRNA may be employed as an inhibitor of an lncRNA selected from SEQ ID NOs 1 to 14 after introduction into the respective cells.

The term “antisense nucleic acid molecule”, as used herein, refers to a nucleic acid which is complementary to a target nucleic acid. An antisense molecule in accordance with the invention is capable of interacting with the target nucleic acid, more specifically it is capable of hybridizing with the target nucleic acid. Due to the formation of the hybrid, transcription of the target gene(s) and/or translation of the target mRNA is reduced or blocked. Standard methods relating to antisense technology have been described (see, e.g., Melani et al., Cancer Res. (1991) 51:2897-2901).

The antisense nucleic acid molecule is preferably a GapmeR, an Antagomir, or an antimir and is most preferably a GapmerR, noting that they are used in the appended examples.

LNA-GapmeRs or simply GapmeRs are potent antisense oligonucleotides used for highly efficient inhibition of mRNA and lncRNA function. GapmeRs function by RNase H dependent degradation of complementary RNA targets. They are an excellent alternative to siRNA for knockdown of mRNA and lncRNA. They are advantageously taken up by cell without transfection reagents. GapmeRs contain a central stretch of DNA monomers flanked by blocks of LNAs. The GapmeRs are preferably 14-16 nucleotides in length and are optionally fully phosphorothioated. The DNA gap activates the RNAse H-mediated degradation of targeted RNAs and is also suitable to target transcripts directly in the nucleus. The LNA-GapmeR according to the invention comprises a sequence which is with increasing preference complementary to at least 13 nucleotides, at least 14 nucleotides, or at least 15 nucleotides of one selected from SEQ ID NOs 1 to 14. These at least 13 nucleotides, at least 14 nucleotides, or at least 15 nucleotides are preferably a contiguous part of one selected from SEQ ID NOs 1 to 14, i.e. the nucleotides are consecutive in the respective SEQ ID NO. The LNA-GapmeR technology is well established. LNA-GapmeRs are routinely designed using established algorithms. LNA-GapmeRs to a selected target are commercially available including positive and negative controls, for example, from Exiqon.

The sequences of the Gapmers that are used in the examples are shown in the following Table in the second column and in SEQ ID NOs 18 to 33.

| LncRNA | 5′-3′ |
|---|---|
| lncRNA 1.1 | AATTACCGTGCTTACC |
| lncRNA 1.2 | GAAACTCCTTCGCGTG |
| lncRNA 4.1 | GCTAATCTATGAGTGA |
| lncRNA 4.3 | CCAGAATGGCAGTAA |
| lncRNA 7.1 | TAGTAACTCTGTAGGA |
| lncRNA 7.2 | ACGCAGCAGATAAAAT |
| lncRNA 7.3 | GTAGTAACTCTGTAGG |
| lncRNA 7.4 | ACAGAGGTGTCGATTA |
| lncRNA 8.1 | TACGCTGAGTGGATGA |
| lncRNA 8.2 | TTAGCGAATGATTAA |
| lncRNA 10.1 | GACTCGGCTAAGATAA |
| lncRNA 10.2 | GAAATGCCGTTAGAGT |
| lncRNA 11.1 | CGTGTGGATCAATGAG |
| lncRNA 11.2 | TGAGTAGCATGCAATC |
| lncRNA 13.1 | CAGTAACGTCTTCTTA |
| lncRNA 13.2 | ACTTGAAGCCTGACTA |

The above Gapmers are particularly preferred. At least two Gapers for each lncRNAs were used. FIG. 9 shows that the GapmeRs #10.1 and #11.1 resulted in a strong knockdown of the lncRNAs SEQ ID NOs 2 and 1, respectively, in heart cells. Gapmers #10.1 and #11.1 are preferred as compared to Gapmers #10.2 and #11.2

As mentioned, AntimiRs are oligonucleotide inhibitors that were initially designed to be complementary to a miRNA. AntimiRs against miRNAs have been used extensively as tools to gain understanding of specific miRNA functions and as potential therapeutics. As used herein, the AntimiRs are designed to be complementary to an lncRNA selected from SEQ ID NOs 1 to 14. AntimiRs are preferably 14 to 23 nucleotides in length. An AntimiR according to the invention more preferably comprises or consists a sequence which is with increasing preference complementary to at least 15 nucleotides, at least 16 nucleotides, at least 17 nucleotides, at least 18 nucleotides, at least 19 nucleotides, at least 20 nucleotides, at least 21 nucleotides, at least 22 nucleotides, or at least 23 nucleotides of one selected from SEQ ID NOs 1 to 14. These at least 15 nucleotides, at least 16 nucleotides, at least 17 nucleotides, at least 18 nucleotides, at least 19 nucleotides, at least 20 nucleotides, at least 21 nucleotides, at least 22 nucleotides, or at least 23 nucleotides are preferably a contiguous part of one selected from SEQ ID NOs 1 to 14, i.e. the nucleotides are consecutive in the respective SEQ ID NO.

AntimiRs are preferably AntagomiRs. AntagomiRs are synthetic 2-O-methyl RNA oligonucleotides, preferably of 21 to 23 nucleotides which are preferably fully complementary to the selected target RNA. While AntagomiRs were initially designed against miRNAs they may also be designed against lncRNAs. The AntagomiRs according to the invention therefore preferably comprises a sequence being complementary to 21 to 23 nucleotides of one of SEQ ID NOs 1 to 14. These 21 to 23 nucleotides are preferably complementary to a contiguous part of one of SEQ ID NOs 1 to 14 including the preferred target sequences within SEQ ID NOs 1 to 14 described herein above, i.e. the nucleotides are consecutive in the respective SEQ ID NO. AntagomiRs are preferably synthesized with 2′-OMe modified bases (2′-hydroxyl of the ribose is replaced with a methoxy group), phosphorothioate (phosphodiester linkages are changed to phosphorothioates) on the first two and last four bases, and an addition of cholesterol motif at 3′ end through a hydroxyprolinol modified linkage. The addition of 2′-OMe and phosphorothioate modifications improve the bio-stability whereas cholesterol conjugation enhances distribution and cell permeation of the AntagomiRs.

Antisense molecules (including antisense oligonucleotides, such as LNA-GapmeR, an Antagomir, an antimiR), siRNAs and shRNAs of the present invention are preferably chemically synthesized using a conventional nucleic acid synthesizer. Suppliers of nucleic acid sequence synthesis reagents include Proligo (Hamburg, Germany), Dharmacon Research (Lafayette, CO, USA), Pierce Chemical (part of Perbio Science, Rockford, IL, USA), Glen Research (Sterling, VA, USA), ChemGenes (Ashland, MA, USA), and Cruachem (Glasgow, UK).

The ability of antisense molecules (including antisense oligonucleotides, such as LNA-GapmeR, an Antagomir, an antimiR), siRNA, and shRNA to potently, but reversibly, silence or inhibit a lncRNA in vivo makes these molecules particularly well suited for use in the pharmaceutical composition of the invention. Ways of administering siRNA to humans are described in De Fougerolles et al., Current Opinion in Pharmacology, 2008, 8:280-285. Such ways are also suitable for administering other small RNA molecules like antisense oligonucleotides or shRNAs. Accordingly, such pharmaceutical compositions may be administered directly formulated as a saline, via liposome based and polymer-based nanoparticle approaches, as conjugated or complexation pharmaceutical compositions, or via viral delivery systems. Direct administration comprises injection into tissue, intranasal and intratracheal administration. Liposome based and polymer-based nanoparticle approaches comprise the cationic lipid Genzyme Lipid (GL) 67, cationic liposomes, chitosan nanoparticles and cationic cell penetrating peptides (CPPs). Conjugated or complexation pharmaceutical compositions comprise PEI-complexed antisense molecules (including antisense oligonucleotides), siRNA, or shRNA. Further, viral delivery systems comprise influenza virus envelopes and virosomes.

The antisense molecules (including antisense oligonucleotides, such as LNA-GapmeR, an Antagomir, an antimiR), siRNAs, shRNAs may comprise modified nucleotides such as locked nucleic acids (LNAs). The ribose moiety of an LNA nucleotide is modified with an extra bridge connecting the 2′ oxygen and 4′ carbon. The bridge “locks” the ribose in the 3′-endo (North) conformation, which is often found in the A-form duplexes. LNA nucleotides can be mixed with DNA or RNA residues in the oligonucleotide whenever desired. Such oligomers are synthesized chemically and are commercially available. The locked ribose conformation enhances base stacking and backbone pre-organization. This significantly increases the hybridization properties (melting temperature) of oligonucleotides.

A ribozyme (from ribonucleic acid enzyme, also called RNA enzyme or catalytic RNA) is an RNA molecule that catalyses a chemical reaction. Many natural ribozymes catalyse either their own cleavage or the cleavage of other RNAs, but they have also been found to catalyse the aminotransferase activity of the ribosome. Non-limiting examples of well-characterised small self-cleaving RNAs are the hammerhead, hairpin, hepatitis delta virus, and in vitro-selected lead-dependent ribozymes, whereas the group I intron is an example for larger ribozymes. The principle of catalytic self-cleavage has become well established in recent years. The hammerhead ribozymes are characterised best among the RNA molecules with ribozyme activity. Since it was shown that hammerhead structures can be integrated into heterologous RNA sequences and that ribozyme activity can thereby be transferred to these molecules, it appears that catalytic antisense sequences for almost any target sequence can be created, provided the target sequence contains a potential matching cleavage site. The basic principle of constructing hammerhead ribozymes is as follows: A region of interest of the RNA, which contains the GUC (or CUC) triplet, is selected. Two oligonucleotide strands, each usually with 6 to 8 nucleotides, are taken and the catalytic hammerhead sequence is inserted between them. The best results are usually obtained with short ribozymes and target sequences.

A recent development, also useful in accordance with the present invention, is the combination of an aptamer, recognizing a small compound, with a hammerhead ribozyme. The conformational change induced in the aptamer upon binding the target molecule can regulate the catalytic function of the ribozyme.

CRISPR/Cas9, as well as CRISPR-Cpf1, technologies are applicable in nearly all cells/model organisms and can be used for knock-out mutations, chromosomal deletions, editing of DNA sequences and regulation of gene expression. The regulation of the gene expression can be manipulated by the use of a catalytically dead Cas9 enzyme (dCas9) that is conjugated with a transcriptional repressor to repress transcription a specific gene, here an lncRNA selected from SEQ ID NOs 1 to 14. Similarly, catalytically inactive, “dead” Cpf1 nuclease (CRISPR from Prevotella and Francisella-1) can be fused to synthetic transcriptional repressors or activators to downregulate endogenous promoters, e.g. the promoter which controls lncRNA expression. Alternatively, the DNA-binding domain of zinc finger nucleases (ZFNs) or transcription activator-like effector nucleases (TALENs) can be designed to specifically recognize the lncRNA gene or its promoter region or its 5′-UTR thereby inhibiting the expression of the lncRNA gene.

Inhibitors provided as inhibiting nucleic acid molecules that target the lncRNA gene or a regulatory molecule involved in lncRNA expression are also envisaged herein. Such molecules, which reduce or abolish the expression of an lncRNA or a regulatory molecule include, without being limiting, meganucleases, zinc finger nucleases and transcription activator-like (TAL) effector (TALE) nucleases. Such methods are described in Silva et al., Curr Gene Ther. 2011; 11(1):11-27; Miller et al., Nature biotechnology. 2011; 29(2):143-148, and Klug, Annual review of biochemistry. 2010; 79:213-231.

The term “antibody” as used in accordance with the present invention comprises, for example, polyclonal or monoclonal antibodies. Furthermore, also derivatives or fragments thereof, which still retain the binding specificity to the target, e.g. an lncRNA selected from SEQ ID NOs 1 to 14, are comprised in the term “antibody”. Antibody fragments or derivatives comprise, inter alia, Fab or Fab′ fragments, Fd, F(ab′)2, Fv or scFv fragments, single domain VH or V-like domains, such as VhH or V-NAR-domains, as well as multimeric formats such as minibodies, diabodies, tribodies or triplebodies, tetrabodies or chemically conjugated Fab′-multimers (see, for example, Harlow and Lane “Antibodies, A Laboratory Manual”, Cold Spring Harbor Laboratory Press, 198; Harlow and Lane “Using Antibodies: A Laboratory Manual” Cold Spring Harbor Laboratory Press, 1999; Altshuler E P, Serebryanaya D V, Katrukha A G. 2010, Biochemistry (Mosc)., vol. 75(13), 1584; Holliger P, Hudson P J. 2005, Nat Biotechnol., vol. 23(9), 1126). The multimeric formats in particular comprise bispecific antibodies that can simultaneously bind to two different types of antigen. The first antigen can be found on the protein of the invention. The second antigen may, for example, be a tumor marker that is specifically expressed on cancer cells or a certain type of cancer cells. Non-limiting examples of bispecific antibodies formats are Biclonics (bispecific, full length human IgG antibodies), DART (Dual-affinity Re-targeting Antibody) and BiTE (consisting of two single-chain variable fragments (scFvs) of different antibodies) molecules (Kontermann and Brinkmann (2015), Drug Discovery Today, 20(7):838-847).

The term “antibody” also includes embodiments such as chimeric (human constant domain, non-human variable domain), single chain and humanised (human antibody with the exception of non-human CDRs) antibodies.

Various techniques for the production of antibodies are well known in the art and described, e.g. in Harlow and Lane (1988) and (1999) and Altshul et al., 2010, loc. cit. Thus, polyclonal antibodies can be obtained from the blood of an animal following immunization with an antigen in mixture with additives and adjuvants and monoclonal antibodies can be produced by any technique which provides antibodies produced by continuous cell line cultures. Examples for such techniques are described, e.g. in Harlow E and Lane D, Cold Spring Harbor Laboratory Press, 1988; Harlow E and Lane D, Using Antibodies: A Laboratory Manual, Cold Spring Harbor Laboratory Press, 1999 and include the hybridoma technique originally described by Köhler and Milstein, 1975, the trioma technique, the human B-cell hybridoma technique (see e.g. Kozbor D, 1983, Immunology Today, vol. 4, 7; Li J, et al. 2006, PNAS, vol. 103(10), 3557) and the EBV-hybridoma technique to produce human monoclonal antibodies (Cole et al., 1985, Alan R. Liss, Inc, 77-96). Furthermore, recombinant antibodies may be obtained from monoclonal antibodies or can be prepared de novo using various display methods such as phage, ribosomal, mRNA, or cell display. A suitable system for the expression of the recombinant (humanised) antibodies may be selected from, for example, bacteria, yeast, insects, mammalian cell lines or transgenic animals or plants (see, e.g., U.S. Pat. No. 6,080,560; Holliger P, Hudson P J. 2005, Nat Biotechnol., vol. 23(9), 11265). Further, techniques described for the production of single chain antibodies (see, inter alia, U.S. Pat. No. 4,946,778) can be adapted to produce single chain antibodies specific for an epitope of an lncRNA selected from SEQ ID NOs 1 to 14. Surface plasmon resonance as employed in the BIAcore system can be used to increase the efficiency of phage antibodies.

As used herein, the term “antibody mimetics” refers to compounds which, like antibodies, can specifically bind antigens, such the an lncRNA selected from SEQ ID NOs 1 to 14 in the present case, but which are not structurally related to antibodies. Antibody mimetics are usually artificial peptides or proteins with a molar mass of about 3 to 20 kDa. For example, an antibody mimetic may be selected from the group consisting of affibodies, adnectins, anticalins, DARPins, avimers, nanofitins, affilins, Kunitz domain peptides, Fynomers®, trispecific binding molecules and prododies. These polypeptides are well known in the art and are described in further detail herein below.

The term “affibody”, as used herein, refers to a family of antibody mimetics which is derived from the Z-domain of staphylococcal protein A. Structurally, affibody molecules are based on a three-helix bundle domain which can also be incorporated into fusion proteins. In itself, an affibody has a molecular mass of around 6 kDa and is stable at high temperatures and under acidic or alkaline conditions. Target specificity is obtained by randomisation of 13 amino acids located in two alpha-helices involved in the binding activity of the parent protein domain (Feldwisch J, Tolmachev V.; (2012) Methods Mol Biol. 899:103-26).

The term “adnectin” (also referred to as “monobody”), as used herein, relates to a molecule based on the 10th extracellular domain of human fibronectin Ill (10Fn3), which adopts an Ig-like β-sandwich fold of 94 residues with 2 to 3 exposed loops, but lacks the central disulphide bridge (Gebauer and Skerra (2009) Curr Opinion in Chemical Biology 13:245-255). Adnectins with the desired target specificity, i.e. against an lncRNA selected from SEQ ID NOs 1 to 14, can be genetically engineered by introducing modifications in specific loops of the protein.

The term “anticalin”, as used herein, refers to an engineered protein derived from a lipocalin (Beste G, Schmidt F S, Stibora T, Skerra A. (1999) Proc Natl Acad Sci USA. 96(5):1898-903; Gebauer and Skerra (2009) Curr Opinion in Chemical Biology 13:245-255). Anticalins possess an eight-stranded β-barrel which forms a highly conserved core unit among the lipocalins and naturally forms binding sites for ligands by means of four structurally variable loops at the open end. Anticalins, although not homologous to the IgG superfamily, show features that so far have been considered typical for the binding sites of antibodies: (i) high structural plasticity as a consequence of sequence variation and (ii) elevated conformational flexibility, allowing induced fit to targets with differing shape.

As used herein, the term “DARPin” refers to a designed ankyrin repeat domain (166 residues), which provides a rigid interface arising from typically three repeated β-turns. DARPins usually carry three repeats corresponding to an artificial consensus sequence, wherein six positions per repeat are randomised. Consequently, DARPins lack structural flexibility (Gebauer and Skerra, 2009).

The term “avimer”, as used herein, refers to a class of antibody mimetics which consist of two or more peptide sequences of 30 to 35 amino acids each, which are derived from A-domains of various membrane receptors and which are connected by linker peptides. Binding of target molecules occurs via the A-domain and domains with the desired binding specificity, i.e. for an lncRNA selected from SEQ ID NOs 1 to 14, can be selected, for example, by phage display techniques. The binding specificity of the different A-domains contained in an avimer may, but does not have to be identical (Weidle U H, et al., (2013), Cancer Genomics Proteomics; 10(4):155-68).

A “nanofitin” (also known as affitin) is an antibody mimetic protein that is derived from the DNA binding protein Sac7d of Sulfolobus acidocaldarius. Nanofitins usually have a molecular weight of around 7 kDa and are designed to specifically bind a target molecule, such as e.g. an lncRNA selected from SEQ ID NOs 1 to 14, by randomising the amino acids on the binding surface (Mouratou B, Behar G, Paillard-Laurance L, Colinet S, Pecorari F., (2012) Methods Mol Biol.; 805:315-31).

The term “affilin”, as used herein, refers to antibody mimetics that are developed by using either gamma-B crystalline or ubiquitin as a scaffold and modifying amino-acids on the surface of these proteins by random mutagenesis. Selection of affilins with the desired target specificity, i.e. against an lncRNA selected from SEQ ID NOs 1 to 14, is effected, for example, by phage display or ribosome display techniques. Depending on the scaffold, affilins have a molecular weight of approximately 10 or 20 kDa. As used herein, the term affilin also refers to di- or multimerised forms of affilins (Weidle U H, et al., (2013), Cancer Genomics Proteomics; 10(4):155-68).

A “Kunitz domain peptide” is derived from the Kunitz domain of a Kunitz-type protease inhibitor such as bovine pancreatic trypsin inhibitor (BPTI), amyloid precursor protein (APP) or tissue factor pathway inhibitor (TFPI). Kunitz domains have a molecular weight of approximately 6kDA and domains with the required target specificity, i.e. against an lncRNA selected from SEQ ID NOs 1 to 14, can be selected by display techniques such as phage display (Weidle et al., (2013), Cancer Genomics Proteomics; 10(4):155-68).

As used herein, the term “Fynomer®” refers to a non-immunoglobulin-derived binding polypeptide derived from the human Fyn SH3 domain. Fyn SH3-derived polypeptides are well-known in the art and have been described e.g. in Grabulovski et al. (2007) JBC, 282, p. 3196-3204, WO 2008/022759, Bertschinger et al (2007) Protein Eng Des Sel 20(2):57-68, Gebauer and Skerra (2009) Curr Opinion in Chemical Biology 13:245-255, or Schlatter et al. (2012), MAbs 4:4, 1-12).

The term “trispecific binding molecule” as used herein refers to a polypeptide molecule that possesses three binding domains and is thus capable of binding, preferably specifically binding to three different epitopes. At least one of these three epitopes is an epitope of the protein of the fourth aspect of the invention. The two other epitopes may also be epitopes of the protein of the fourth aspect of the invention or may be epitopes of one or two different antigens. The trispecific binding molecule is preferably a TriTac. A TriTac is a T-cell engager for solid tumors which comprised of three binding domains being designed to have an extended serum half-life and be about one-third the size of a monoclonal antibody.

As used herein, the term “probody” refers to a protease-activatable antibody prodrug. A probody consists of an authentic IgG heavy chain and a modified light chain. A masking peptide is fused to the light chain through a peptide linker that is cleavable by tumor-specific proteases. The masking peptide prevents the probody binding to healthy tissues, thereby minimizing toxic side effects.

In accordance with a further preferred embodiment of the first and second aspect of the invention the nucleotide-based inhibitor comprises (a) a nucleic acid sequence which comprises or consists of a nucleic acid sequence being complementary to at least 12 continuous nucleotides of a nucleic acid sequence selected from SEQ ID NOs 1 to 14, (b) a nucleic acid sequence which comprises or consists of a nucleic acid sequence which is at least 70% identical to the complementary strand of one or more nucleic acid sequences selected from SEQ ID NOs 1 to 14, (c) a nucleic acid sequence which comprises or consists of a nucleic acid sequence according to (a) or (b), wherein the nucleic acid sequence is DNA or RNA, (d) an expression vector expressing the nucleic acid sequence as defined in any one of (a) to (c), preferably under the control of a heart-specific promoter and/or a fibroblast and/or fibrocyte-specific promoter, or (e) a host comprising the expression vector of (d).

The nucleic acid sequences as defined in items (a) to (c) of this preferred embodiment comprise or consist of sequences being complementary to nucleotides of an lncRNA selected from SEQ ID NOs 1 to 14. Hence, the nucleic acid sequences as defined in items (a) to (c) comprise or are antisense nucleic acid sequences.

The nucleic acid sequence according to item (a) of this further preferred embodiment of the invention comprises or consists of a sequence which is with increasing preference complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides of an lncRNA selected from SEQ ID NOs 1 to 14. These at least 13 nucleotides, at least 14 nucleotides, at least 15 nucleotides, at least 16 nucleotides, at least 17 nucleotides, at least 18 nucleotides, at least 19 nucleotides, at least 20 nucleotides, or at least 21 nucleotides are preferably a contiguous part of an lncRNA selected from SEQ ID NOs 1 to 14, i.e. the nucleotides are consecutive in the respective SEQ ID NO. The format of the nucleic acid sequence according to item (a) is not particularly limited as long as it comprises or consists of at least 12 continuous nucleotides being complementary to a nucleic acid sequence selected from SEQ ID NOs 1 to 14. The nucleic acid sequence according to item (a) comprises or consists of an antisense oligonucleotide. Hence, the nucleic acid sequence according to item (a) reflects the above-mentioned basic principle of the antisense technology which is the use of an oligonucleotide for silencing a selected target RNA through the exquisite specificity of complementary-based pairing. Therefore, it is to be understood that the nucleic acid sequence according to item (a) is preferably in the format of an siRNA, shRNA or an antisense oligonucleotide as defined herein above. The antisense oligonucleotides are preferably LNA-GapmeRs, AntagomiRs, or antimiRs as defined herein above. LNA-GapmeRs are again preferred, particularly since they are used in the examples.

The nucleic acid sequence according to item (b) requiring at least 70% identity to the complementary strand of an lncRNA selected from SEQ ID NOs 1 to 14 is considerably longer than the nucleic acid sequence according to item (a) which comprises an antisense oligonucleotide and comprises at least 12 continuous nucleotides of an lncRNA selected from SEQ ID NOs 1 to 14. A nucleic acid sequence according to items (a) and (b) of the above preferred embodiment of the invention is capable of interacting with, more specifically hybridizing with the target lncRNA. By formation of the hybrid the function of the lncRNA is reduced or blocked.

The sequence identity of the molecule according to item (b) in connection with a sequence selected from SEQ ID NOs 1 to 14 is with increasing preference at least 75%, at least 80%, at least 85%, at least 90%, at least 92.5%, at least 95%, at least 98%, at least 99% and 100%. The sequence identity in connection with each of SEQ ID NOs 1 to 14 can be individually selected. For instance, a non-limiting example is at least 90% in connection with SEQ ID NO: 1 and at least 95% in connection with SEQ ID NO: 2. Means and methods for determining sequence identity are known in the art. Preferably, the BLAST (Basic Local Alignment Search Tool) program is used for determining the sequence identity with regard to one or more of SEQ ID NOs 1 to 14.

In the nucleic acid sequence according to item (c) the nucleotide sequences may be RNA or DNA. RNA or DNA optionally encompasses chemically modified RNA nucleotides or DNA nucleotides. As commonly known RNA comprises the nucleotide U while DNA comprises the nucleotide T.

In accordance with items (d) and (e) of the above preferred embodiment the inhibitor may also be an expression vector or host, respectively being capable of producing a nucleic acid sequence as defined in any one of items (a) to (c).

An expression vector may be a plasmid that is used to introduce a specific transcript into a target cell. Once the expression vector is inside the cell, the protein that is encoded by the gene is produced by the cellular-transcription and translation machinery ribosomal complexes. The plasmid is in general engineered to contain regulatory sequences that act as enhancer and/or promoter regions and lead to efficient transcription of the transcript. In accordance with the present invention the expression vector preferably contains a heart-specific promoter and/or a fibroblast and/or fibrocyte-specific promoter, wherein the fibroblast and/or fibrocyte-specific promoter is preferably a fibroblast-specific promoter. Heart-specific promoters are known in the art, for example, from Boecker at al. (2004), Mol Imagin.; 3(2):69-75. Using a heart-specific promoter ensures that the nucleic acid sequence is only expressed in the heart and may avoid potential unwanted side effects by expression in other organs. Promoters for fibroblast-specific expression are as well known in the art, e.g., from Takeda et al (2010), J Clin Invest. 2010; 120(1):254-265 or Hemmings et al. (2014), Heart; 100:A19-A20. Using a fibroblast and/or fibrocyte-specific promoter ensures that the nucleic acid sequence is only expressed in fibroblasts and/or fibrocytes and may avoid potential unwanted side effects by expression in other cell-types, such as endothelial cells. The fibroblast-specific promoter described in Takeda et al (2010), loc. lit. is in particular specific for heart fibroblasts.

Accordingly, also promoters being heart-specific and a fibroblast-specific are known and are most preferably used as a promoter in the context of the present invention. This is because such a promoter may avoid potential unwanted side effects by expression in other organs as well as in other cell-types.

Non-limiting examples of expression vectors include prokaryotic plasmid vectors, such as the pUC-series, pBluescript (Stratagene), the pET-series of expression vectors (Novagen) or pCRTOPO (Invitrogen) and vectors compatible with an expression in mammalian cells like pREP (Invitrogen), pcDNA3 (Invitrogen), pCEP4 (Invitrogen), pMC1neo (Stratagene), pXT1 (Stratagene), pSG5 (Stratagene), EBO-pSV2neo, pBPV-1, pdBPVMMTneo, pRSVgpt, pRSVneo, pSV2-dhfr, pIZD35, pLXIN, pSIR (Clontech), pIRES-EGFP (Clontech), pEAK-10 (Edge Biosystems) pTriEx-Hygro (Novagen) and pCINeo (Promega). Examples for plasmid vectors suitable for Pichia pastoris comprise e.g. the plasmids pAO815, pPIC9K and pPIC3.5K (all Intvitrogen). For the formulation of a pharmaceutical composition a suitable vector is selected in accordance with good manufacturing practice. Such vectors are known in the art, for example, from Ausubel et al, Hum Gene Ther. 2011 April; 22(4):489-97 or Allay et al., Hum Gene Ther. May 2011; 22(5): 595-604.

A typical mammalian expression vector contains the promoter element, which mediates the initiation of transcription of mRNA, the protein coding sequence, and signals required for the termination of transcription and polyadenylation of the transcript. Moreover, elements such as origin of replication, drug resistance gene, regulators (as part of an inducible promoter) may also be included. The lac promoter is atypical inducible promoter, useful for prokaryotic cells, which can be induced using the lactose analogue isopropylthiol-b-D-galactoside (“IPTG”). For recombinant expression and secretion, the polynucleotide of interest may be ligated between e.g. the PeIB leader signal, which directs the recombinant protein in the periplasm and the gene Ill in a phagemid called pHEN4 (described in Ghahroudi et al, 1997, FEBS Letters 414:521-526). Additional elements might include enhancers, Kozak sequences and intervening sequences flanked by donor and acceptor sites for RNA splicing. Highly efficient transcription can be achieved with the early and late promoters from SV40, the long terminal repeats (LTRs) from retroviruses, e.g., RSV, HTLVI, HIVI, and the early promoter of the cytomegalovirus (CMV). However, cellular elements can also be used (e.g., the human actin promoter). Suitable expression vectors for use in practicing the present invention include, for example, vectors such as pSVL and pMSG (Pharmacia, Uppsala, Sweden), pRSVcat (ATCC 37152), pSV2dhfr (ATCC 37146) and pBC12MI (ATCC 67109). Alternatively, the recombinant (poly)peptide can be expressed in stable cell lines that contain the gene construct integrated into a chromosome. The co-transfection with a selectable marker such as dhfr, gpt, neomycin, hygromycin allows the identification and isolation of the transfected cells. The transfected nucleic acid can also be amplified to express large amounts of the encoded (poly)peptide. The DHFR (dihydrofolate reductase) marker is useful to develop cell lines that carry several hundred or even several thousand copies of the gene of interest. Another useful selection marker is the enzyme glutamine synthase (GS) (Murphy et al.1991, Biochem J. 227:277-279; Bebbington et al. 1992, Bio/Technology 10:169-175). Using these markers, the mammalian cells are grown in selective medium and the cells with the highest resistance are selected. As indicated above, the expression vectors will preferably include at least one selectable marker.

Such markers include dihydrofolate reductase, G418 or neomycin resistance for eukaryotic cell culture and tetracycline, kanamycin or ampicillin resistance genes for culturing in E. coli and other bacteria. For vector modification techniques, see Sambrook and Russel (2001), Molecular Cloning: A Laboratory Manual, 3 Vol. Generally, vectors can contain one or more origins of replication (ori) and inheritance systems for cloning or expression, one or more markers for selection in the host, e.g., antibiotic resistance, and one or more expression cassettes. Suitable origins of replication (ori) include, for example, the Col E1, the SV40 viral and the M 13 origins of replication.

The sequences to be inserted into the vector can e.g. be synthesized by standard methods, or isolated from natural sources. Ligation of the coding sequences to transcriptional regulatory elements and/or to other amino acid encoding sequences can be carried out using established methods. Transcriptional regulatory elements (parts of an expression cassette) ensuring expression in prokaryotes or eukaryotic cells are well known to those skilled in the art. These elements comprise regulatory sequences ensuring the initiation of the transcription (e.g., translation initiation codon, promoters, enhancers, and/or insulators), internal ribosomal entry sites (IRES) (Owens, Proc. Natl. Acad. Sci. USA 98 (2001), 1471-1476) and optionally poly-A signals ensuring termination of transcription and stabilization of the transcript. Additional regulatory elements may include transcriptional as well as translational enhancers, and/or naturally-associated or heterologous promoter regions. Preferably, the nucleotide sequence as defined in item (a) of the above preferred embodiment of the invention is operatively linked to such expression control sequences allowing expression in prokaryotic or eukaryotic cells. The host may be a prokaryotic or eukaryotic cell. A suitable eukaryotic host may be a mammalian cell, an amphibian cell, a fish cell, an insect cell, a fungal cell or a plant cell. Representative examples of bacterial cells are E. coli, Streptomyces and Salmonella typhimurium cells; of fungal cells are yeast cells; and of insect cells are Drosophila S2 and Spodoptera Sf9 cells. It is preferred that the cell is a mammalian cell such as a human cell. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells. The cell may be a part of a cell line, preferably a human cell line. Appropriate culture mediums and conditions for the above-described host cells are known in the art. The host is preferably a host cell and more preferably an isolated host cell. The host is also preferably a non-human host.

In accordance with another preferred embodiment of the first and second aspect of the invention the compound as defined in (ii) is (a) a nucleic acid sequence which comprises or consists of the nucleic acid sequence of one or more lncRNAs selected from SEQ ID NOs 15 to 17 or a nucleic acid sequence which is at least 70%, preferably at least 80% and most preferably at least 90% identical thereto, (b) an expression vector expressing the nucleic acid sequence as defined in (a), preferably under the control of a heart-specific promoter and/or a fibroblast and/or fibrocyte-specific promoter, or (c) a host comprising the expression vector of (b).

The nucleic acid sequence according to item (a) of this preferred embodiment may be a recombinantly produced or isolated lncRNAs selected from SEQ ID NOs 15 to 17, any precursor thereof or any fragment thereof as long as a sequence identity of at least 70% over the entire length of an lncRNA selected from SEQ ID NOs 15 to 17 is maintained. Also orthologous or homologous sequences of the lncRNA selected from SEQ ID NOs 15 to 17 from different species including precursor or a functional fragment thereof may be used. The fragments have to retain or essentially retain the function of the full-length lncRNA. Hence, the fragments have to be active/functional fragments.

The sequence identity of the nucleic acid sequence according to item (a) to an lncRNA selected from SEQ ID NOs 15 to 17 is with increasing preference at least 75%, at least 80%, at least 85%, at least 90%, at least 95%, at least 98%, at least 99% and 100%. Means and methods for determining sequence identity are known in the art. Preferably, the BLAST (Basic Local Alignment Search Tool) program is used for determining the sequence identity with regard to one or more lncRNAs selected from SEQ ID NOs 15 to 17.

In accordance with items (b) and (c) of the above preferred embodiment such a compound may also be an expression vector or host being capable of producing a nucleic acid sequence as defined in item (a). Suitable expression vectors or hosts have been described herein above in connection with compound inhibiting the expression and/or the activity of one or more lncRNAs.

In accordance with a further preferred embodiment of the first and second aspect of the invention the compound as defined in (ii) is (a) a transcription factor promoting the expression of one or more lncRNAs selected from SEQ ID NOs 15 to 17, and/or (b) a small molecule enhancing the expression of one or more lncRNAs selected from SEQ ID NOs 15 to 17.

The term “transcription factor” as used herein defines a protein or peptide that binds to specific DNA sequences, thereby controlling the transcription of the genes encoding of one or more lncRNAs selected from SEQ ID NOs 15 to 17. The efficiency of a transcription factor in activating the expression of an lncRNAs selected from SEQ ID NOs 15 to 17 can be quantified by methods comparing the level of the lncRNA in the presence of the transcription factor to that in the absence of the transcription factor. For example, as an activity measure the change in amount of lncRNA formed may be used. Such a method may be effected in high-throughput format in order to test the efficiency of several inhibiting compound simultaneously. High-throughput formats have been further detailed herein above.

The small molecule enhancing the expression of one or more lncRNAs selected from SEQ ID NOs 15 to 17 is a low molecular weight organic compound which is by definition not a polymer. The small molecule of the invention is preferably a molecule that binds with high affinity to an lncRNA of SEQ ID NOs 15 to 17 and in addition enhances the activity of an lncRNA of SEQ ID NOs 15 to 17. The upper molecular weight limit for a small molecule is preferably 1500 Da, more preferably 1000 Da and most preferably 800 Da which allows for the possibility to rapidly diffuse across cell membranes so that they can reach intracellular sites of action. Libraries of small organic molecules and high-throughput techniques for screening such libraries with a specific target molecule, in the present case an lncRNA selected from SEQ ID NOs 15 to 17, are established in the art.

The present invention relates in a third aspect to a method for diagnosing a fibrotic disorder or a tumor, preferably cancer in a patient, comprising (a) detecting the expression level of one or more lncRNAs selected from SEQ ID NOs 1 to 17 in a sample obtained from said patient, and (b) comparing said expression level of the one or more lncRNAs with the expression level of these one or more lncRNAs in a sample obtained from healthy subjects or predetermined standard, wherein a greater than 2-fold upregulation of one or more lncRNAs selected from SEQ ID NOs 1 to 14; and/or a greater than 2-fold downregulation of one or more lncRNAs selected from SEQ ID NOs 15 to 17 is indicative for a fibrotic disorder or a tumor, preferably cancer in the patient.

The method according to the third aspect of the invention may also encompass detecting and comparing the expression level of one or more lncRNAs being with increased preference at least 90%, at least 92%, at least 94%, at least 96%, at least 98%, at least 99%, and at least 99.5% identical to any one of SEQ ID NOs 1 to 17. Means and methods for determining sequence identity are known in the art. Preferably, the BLAST (Basic Local Alignment Search Tool) program is used for determining the sequence identity with regard to one or more lncRNAs selected from SEQ ID NOs 1 to 17. The method according to the third aspect of the invention may furthermore encompass detecting and comparing the expression level of one or more lncRNAs differing with increasing preference by no more than 10, such as 5, 4, 3, 2 or 1 nucleotide(s) from any one of SEQ ID NOs 1 to 17. The nucleotide differences may be the addition, deletion and/or substitution of nucleotide(s). The sequences the expression of which is compared, while being homologous, may also differ from each other with increasing preference by no more than 10, such as 5, 4, 3, 2 or 1 nucleotide(s).

The term “sample” designates a tissue sample or a body fluid sample. The body fluid sample is preferably selected from blood, serum, plasma, urine, salvia, amniotic fluid, cerebrospinal fluid and lymph. The tissue sample is preferably an organ sample, such as a heart, liver or kidney sample. As far as the method is applied to a body fluid sample it is to be understood that the expression level of an lncRNA corresponds to the concentration of the lncRNA, because lncRNAs are not directly expressed in the body fluid but secreted from the cells, said cells expressing the lncRNAs, into the body fluids.

The “patient” or “subject” referred to herein is human.

The term “detecting the expression level of lncRNA” means determining the amount or yield of the lncRNA. The lncRNAs are initially expressed within a cell. It was found in accordance with the present invention that the lncRNAs of SEQ ID NOs 1 to 17 can be detected in the sample of a patient, in particular fibrotic samples from a number of different organs. An lncRNA being “expressed in a sample” is therefore a lncRNA whose expression level can be detected in the sample by means and methods being further detailed herein below. An lncRNA is upregulated in a test sample if the amount or yield of the lncRNA is significantly higher (i.e. at least 2-fold and the preferred higher values as described herein below) as compared to the amount or yield of the corresponding lncRNA in a control sample. Likewise, an lncRNA is downregulated in a test sample if the amount or yield of the lncRNA is significantly less (i.e. at least 2-fold and the preferred higher values as described herein below) as compared to the amount or yield of the corresponding lncRNA in a control sample. In this context the term “corresponding lncRNA” means, for example, that the expression level of the lncRNA of SEQ ID NO: 1 in the test sample is compared to the expression level of the lncRNA of SEQ ID NO: 1 in the control sample, or likewise that the expression level of the lncRNA of SEQ ID NO: 2 in the test sample is compared to the expression level of the lncRNA of SEQ ID NO: 2 in the control sample. This applies mutatis mutandis for scenarios where the expression of more than one lncRNA selected from SEQ ID NOs 1 to 17 is determined. For instance, if the expression level of all lncRNAs of SEQ ID NOs 1 to 17 is determined in the test sample it is compared to the expression level of all lncRNAs of SEQ ID NOs 1 to 17 in the control sample.

The expression level in the samples can be quantified by any suitable means and methods available from the art. In general relative and absolute quantification means and methods can be used. In absolute quantification no known standards or controls are needed. The expression level can be directly quantified. As well-known in the art, absolute quantification may rely on a predetermined standard curve. In relative quantification the expression level is quantified relative to a reference (such as known control expressions levels). Also in the absence of controls, one can relatively quantify the expression level when comparing e.g. fluorescence intensities.

Methods to assess RNA concentration may, for example, comprise measuring the fluorescence intensity of dyes that bind to nucleic acids and selectively fluoresce when bound. Such methods comprise a reverse transcription reaction and the production of cDNA, wherein the amount of the cDNA is determined thereby indirectly determining the amount of the RNA. The fluorescent-based method is particularly useful for cases where the RNA concentration is too low to accurately assess some with spectrophotometry and/or in cases where contaminants absorbing at 260 nm make accurate quantification by spectrophotometry difficult or impossible.

When comparing the expression level of the one or more lncRNAs between different samples reliability of the comparison is preferably improved by including an invariant endogenous control (expression of a reference gene) to correct for potential sample to sample variations.

Such normalization with respect to an invariant endogenous control is routinely performed in the art. For example, means and methods for expression level normalization, e.g. in real-time RT-PCR (see, for example, Bustin, Journal of Molecular Endocrinology, (2002) 29, 23-39) or micro-array expression analysis (see, for example, Calza and Balwitan, Methods Mol Biol. 2010; 673:37-52) are well-established. Also methods for normalization of the expression levels of small RNA sequences are established (see, for example, Mestdagh et al. (2009) Genome Biol.; 10(6):R64). In case RT-PCR or a micro-array is used to determine the expression levels in accordance with the present invention, the expression levels are preferably normalized to a spiked-in RNA (see, for example, McCormick et al. (2011), Silence, 2:2). Known amounts of a spiked-in RNA are mixed with the sample during preparation. More preferably the RNA is externally spiked-in to plasma and/or serum before the RNA isolation process is carried out, in which case the samples are plasma and/or serum. The spiked-in RNA technology is well-known and commercial kits are available from a number of manufacturers. The spiked-in RNA is preferably a spiked-in C. elegans RNA.

As evident from the examples herein below and as discussed herein above in connection with the first and second aspect of the invention, the deregulation of the levels of one or more lncRNAs selected from 1 to 15 are indicative for fibrotic disorders and tumors, preferably cancers. Thus, determining the expression levels of one or more of the respective human homologous lncRNAs selected from 1 to 15 is expected to be of prognostic value for diagnosing fibrotic disorders and tumors, preferably cancers in patients. The lncRNAs selected from SEQ ID NOs 1 to 17 may be combined with further diagnostic markers for cardiac hypertrophy in order to enhance the confidentially of the diagnostic method. High-expression level of the lncRNAs selected from SEQ ID NOs 1 to 14 and low expression level of the lncRNAs selected from SEQ ID NOs 15 to 17 is indicative for fibrotic disorders and tumors, preferably cancers.

In the examples herein below particular primer pairs were used for the detection of the 15 lncRNAs. The following Table and SEQ ID NOs 34 to 63 show the primer pairs for each of the lncRNAs.

| lncRNA | lncRNA | lncRNA | | |
|---|---|---|---|---|
| # | gene ID | name | forward primer | reverse primer |
| 1 | ENSG00000227811 | INKA2-AS1 | GGGAGAGAAGGGGAGACAAG | CTCAAAATGCCAGGGAGAAG |
| | | | TGATGAAAGCCACTGAGGAA | CTCAAAATGCCAGGGAGAAG |
| 2 | ENSG00000253819 | LINC01151 | ACATGAACACCCGATCATCA | AAGCAAGAGGAAGGGTTGGT |
| 3 | ENSG00000250056 | LINC01018 | AAATGAGGCTCCTGGGAAAT | TGCACACCTGTGGATGTCTT |
| 4 | ENSG00000251281 | AC034223.2 | CTTCTAAAGGCTGCCTGCAT | GACCTTGGATTATGAGGTGGA |
| 5 | ENSG00000268941 | LINC01711 | ACTCTCCGAGGGTCAAGGAT | CACCCATCAAAGGATGGAAT |
| 6 | ENSG00000262061 | AC129507.1 | ACAGACTGCGCCCTTGTACT | ACGTGAGGCTGCATAATGG |
| 7 | ENSG00000250697 | AC010343.3 | CATCTGCACTGGTGTGAGGT | TATCCAAGTGGGAGCTGCTT |
| | | | CTTCTGGGTCCTCTTGCTTG | CAGGGAGCACACAATCTCCT |
| | | | CTTCTGGGTCCTCTTGCTTG | GATGGAGACTGGCCTTGGTA |
| 8 | ENSG00000259518 | LINC01583 | CCAGAAAGAGGCTGCAAAGT | CAGTTCCTCGATGGCATCTT |
| 9 | ENSG00000234840 | LINC01239 | GGAGAGAAGGAAAGGGAGGA | GGGCTACAATTGACCTCCAG |
| 10 | ENSG00000260604 | AL590004.3 | TGCGTCTCTTCTTGCAGCTA | GCAGTCCCAACTCAGCTTTC |
| | | | GCTCCTTGCAAGTCACCTTC | TTACAAAGCAGCCTGTGTGC |
| 11 | ENSG00000249406 | AC015909.1 | ATTAGAAAACTGAGGCCCAGAG | CAGTATTTTCTTTCACTAGGGGTCAA |
| | | | TAGCTGCCAGAGGTCACACA | ATCTAGCCGCTTCACTCCAA |
| 12 | ENSG00000233901 | LINC01503 | CCCTACCCAGAGACCCTGA | CAGCTACTTGGGAGGCTGAG |
| | | | CCCTACCCAGAGACCCTGA | TGCAAGAGTATTCGGCTGGT |
| | | | CTCCGGGCCCTGATAGAT | ATTTTGAGTGAGGGCTGGAA |
| 13 | ENSG00000248187 | AC078850.1 | TTTCATCCAAGCCTGGAAAT | GGAGCCTGAGTTTCGAAGTG |
| | | | TTTCATCCAAGCCTGGAAAT | AGTCCCTGAGGTTGGTAGGG |
| 14 | ENSG00000251615 | AC104825.1 | ATGGCAGGCTTCAGAGAGAA | GGGACTCCGGTATAGCATCA |
| 15 | ENSG00000249669 | CARMN | CCATGCTGATGTCAGAGAA | CCTCTGTGTTCCCCATGTCT |
| | | | TCAAACTCCTGGGCTCAAGT | GCTCAGCTGGGAATCAGAAC |
| | | | CAGAGGCTGGGTCTAATTAGTTG | GGAGGCTGCTTCTCCAGAGT |

The above primer pair are particularly preferred.

The greater than 2-fold downregulation is with increasing preference greater than 3-fold downregulation, greater than 4-fold downregulation, greater than 5-fold downregulation, greater than 6-fold downregulation, greater than 7-fold downregulation and greater than 8-fold downregulation. Likewise, the greater than 2-fold upregulation is with increasing preference greater than 3-fold upregulation, greater than 4-fold upregulation, greater than 5-fold upregulation, greater than 6-fold upregulation, greater than 7-fold upregulation and greater than 8-fold upregulation. The higher thresholds for the up- and downregulation may increase the reliability of the method of the third aspect of the invention.

In accordance with a preferred embodiment of the third aspect of the invention said sample is a blood sample or blood-derived sample.

The blood-derived sample is preferably plasma or serum.

In accordance with another preferred embodiment of the third aspect of the invention said sample is a tissue sample, preferably a heart tissue sample.

The tissue sample is preferably an organ sample, more preferably a cardiac, pulmonary, liver, kidney, gastrointestinal, or skeletal muscle sample. In case of an organ fibrosis is to be diagnosed it is particularly preferred that the sample is taken from the corresponding organ, such a heart in the case of cardiac fibrosis.

In accordance with a further preferred embodiment of the third aspect of the invention the detection of the expression level of the one or more lncRNAs comprises (a) quantitative PCR, preferably quantitative real time PCR, or (b)a template/RNA amplification method followed by determining the expression level of the one or more lncRNAs using a fluorescence- or luminescence-based quantification method.

In quantitative PCR (qPCR), the amount of amplified product is linked to fluorescence intensity using a fluorescent reporter molecule. The point at which the fluorescent signal is measured in order to calculate the initial template quantity can either be at the end of the reaction (endpoint semi-quantitative PCR) or while the amplification is still progressing (real-time qPCR).

In endpoint semi-quantitative PCR, fluorescence data are collected after the amplification reaction has been completed, usually after 30-40 cycles, and this final fluorescence is used to back-calculate the amount of template present prior to PCR.

The more sensitive and reproducible method of real-time qPCR measures the fluorescence at each cycle as the amplification progresses. This allows quantification of the template to be based on the fluorescence signal during the exponential phase of amplification, before limiting reagents, accumulation of inhibitors, or inactivation of the polymerase have started to have an effect on the efficiency of amplification. Fluorescence readings at these earlier cycles of the reaction will measure the amplified template quantity where the reaction is much more reproducible from sample to sample than at the endpoint.

A non-limiting example of a template/RNA amplification method followed by determining the expression level of the one or more lncRNAs using a fluorescence- or luminescence-based quantification method is a method combining transcription mediated amplification (TMA) and a hybridization protection assay (HPA). In more detail, such a method may comprise hybridizing one or more oligonucleotides (“capture oligonucleotides”) that are complementary to any of SEQ ID NOs 1 to 17. In case two or more of SEQ ID NOs 1 to 17 are targeted, a separate capture oligonucleotide is used for each sequence selected from of SEQ ID NOs 1 to 17. The hybridized target sequences are then captured onto magnetic microparticles that are separated from the sample in a magnetic field. Wash steps may be utilized to remove extraneous components. Target amplification typically occurs via TMA, which is a transcription-based nucleic acid amplification method that utilizes two enzymes, Moloney murine leukemia virus (MMLV) reverse transcriptase and T7 RNA polymerase. A unique set of primers is used for each target sequence selected from of SEQ ID NOs 1 to 17. The reverse transcriptase is used to generate a DNA copy (containing a promoter sequence for T7 RNA polymerase) of the target sequence. T7 RNA polymerase produces multiple copies of RNA amplicon from the DNA copy. Detection of lncRNA expression level is achieved by HPA using single-stranded, chemiluminescent-labeled nucleic acid probes that are complementary to the one or more amplicon. Preferably, distinguishably labelled probes are used for each target amplicon. The labeled nucleic acid probes hybridize specifically to the amplicon. A “selection reagent” then differentiates between hybridized and unhybridized probes by inactivating the label on unhybridized probes. During the detection step, the chemiluminescent signal produced by the hybridized probe is measured in a luminometer and is reported as “Relative Light Units” (RLU), thereby quantifying the lncRNA expression level.

In accordance with a yet further preferred embodiment of the third aspect of the invention the method comprises prior to the detection of the expression level of the long non-coding RNA a pre-amplification step of the RNA within the test patient's sample and/or the control patient's sample.

Performing a pre-amplification step is of particular advantage in case only a low amount of (test and/or control) sample is available. The pre-amplification step allows increasing the amount of RNA within the sample before proceeding to the analysis of the expression level. Means and methods for the pre-amplification of RNA are well known in the art (see, e.g., Vermeulen et al (2009) BMC Res Notes., 2:235). In case both the RNA in the test and control sample is pre-amplified preferably the same method for the pre-amplification step is used such that the relative amount of RNA of the test sample as compared to the control sample is maintained. In case only the RNA of the test or control sample is pre-amplified or the two RNA samples are pre-amplified by different methods, the expression level data may have to be normalized for pre-amplification step; see, e.g. Mestdagh et al. (2009), Genome Biology 2009, 10:R64.

The present invention relates in a fourth aspect to a kit for diagnosing a fibrotic disorder or a tumor, preferably cancer in a patient, said kit comprising means for the detection of the expression level of one or more lncRNAs selected from SEQ ID NOs 1 to 17, and instructions how to use the kit.

The instructions how to use the kit preferably inform inter alia that high-expression level of the lncRNAs selected from SEQ ID NOs 1 to 14 and low expression level of the lncRNAs selected from 15 to 17 is indicative for a fibrotic disorder or a tumor, preferably cancer.

The means for the detection of the expression level of one or more lncRNAs selected from SEQ ID NOs 1 to 17 are preferably the means required for (i) a quantitative PCR, preferably quantitative real time PCR, or (ii) a template/RNA amplification method followed by determining the expression level of the one or more lncRNAs using a fluorescence- or luminescence-based quantification method. These means have been further detailed herein above in connection with the third aspect of the invention and may be comprised in the kit. Hence, the means preferably comprise oligonucleotides, such as fluorescent hybridization probes or primers, which specifically hybridize to one or more lncRNAs selected from SEQ ID NOs 1 to 17. Additional ingredients of the kits may be florescent or luminescent dyes, preferably coupled to said oligonucleotides. Also, additional ingredients of the kits may be enzymes, such as a reverse transcriptase and/or a polymerase.

In accordance with the kit of the invention the means for the detection of the expression level of one or more lncRNAs selected from SEQ ID NOs 1 to 17 preferably comprise means for the detection of the lncRNA of SEQ ID NO: 1 and/or 2, most preferably SEQ ID NO: 1.

The various components of the kit may be packaged in one or more containers such as one or more vials. The vials may, in addition to the components, comprise preservatives or buffers for storage.

In accordance with a preferred embodiment of the fourth aspect of the invention the means are primer pairs used for the specific detection of the expression level of one or more lncRNAs selected from SEQ ID NOs 1 to 17.

Preferred primers and primer pairs are described herein above in connection with the third aspect of the invention. These specific primers are also preferably used in connection with the above preferred embodiment of the fourth aspect of the invention.

In accordance with a preferred embodiment of all aspects of the invention the one or more lncRNAs are at least 2 lncRNAs, and preferably at least 5 lncRNAs.

Employing at least 3 lncRNAs, preferably at least 5 lncRNAs, more preferably at least 10 lncRNAs, and most preferably all lncRNAs of SEQ ID NOs 1 to 17 will additionally increase the effectiveness of the pharmaceutical compositions, medical uses, methods and kits of the invention. Employing these numbers of lncRNAs may balance potential differences associated with particular compounds, probes or methods used in connection with the methods and kits of the invention. In the pharmaceutical compositions and medical uses of the invention these numbers of lncRNAs may increase the beneficial effect for the subject to be treated.

In accordance with a preferred embodiment of all four above-described aspects of the invention the one or more lncRNAs is or comprises the lncRNA(s) of SEQ ID NOs 1 and/or 2, preferably of SEQ ID NO 1.

Regarding the embodiments characterized in this specification, in particular in the claims, it is intended that each embodiment mentioned in a dependent claim is combined with each embodiment of each claim (independent or dependent) said dependent claim depends from. For example, in case of an independent claim 1 reciting 3 alternatives A, B and C, a dependent claim 2 reciting 3 alternatives D, E and F and a claim 3 depending from claims 1 and 2 and reciting 3 alternatives G, H and I, it is to be understood that the specification unambiguously discloses embodiments corresponding to combinations A, D, G; A, D, H; A, D, I; A, E, G; A, E, H; A, E, I; A, F, G; A, F, H; A, F, I; B, D, G; B, D, H; B, D, I; B, E, G; B, E, H; B, E, I; B, F, G; B, F, H; B, F, I; C, D, G; C, D, H; C, D, I; C, E, G; C, E, H; C, E, I; C, F, G; C, F, H; C, F, I, unless specifically mentioned otherwise.

Similarly, and also in those cases where independent and/or dependent claims do not recite alternatives, it is understood that if dependent claims refer back to a majority of preceding claims, any combination of subject-matter covered thereby is considered to be explicitly disclosed. For example, in case of an independent claim 1, a dependent claim 2 referring back to claim 1, and a dependent claim 3 referring back to both claims 2 and 1, it follows that the combination of the subject-matter of claims 3 and 1 is clearly and unambiguously disclosed as is the combination of the subject-matter of claims 3, 2 and 1. In case a further dependent claim 4 is present which refers to any one of claims 1 to 3, it follows that the combination of the subject-matter of claims 4 and 1, of claims 4, 2 and 1, of claims 4, 3 and 1, as well as of claims 4, 3, 2 and 1 is clearly and unambiguously disclosed.

The figures show.

FIG. 1: Differentiation states of cardiac fibroblasts after myocardial infarction in a mouse model.⁸

The examples illustrate the invention.

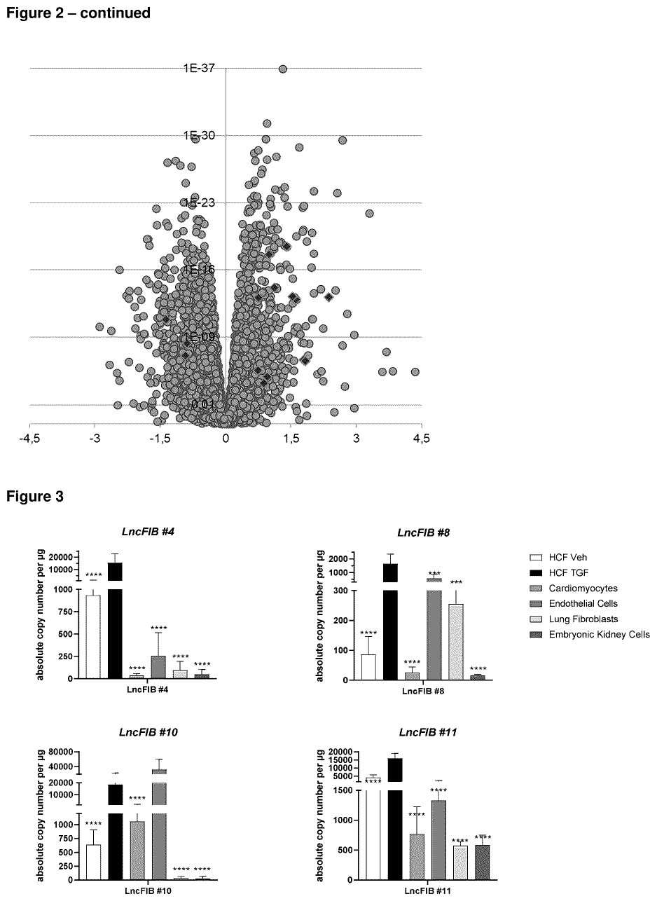

FIG. 2: Scheme of the lncRNA selection from two cohorts (2 and 84 patients). Briefly, cardiac fibroblasts have been isolated by the group of Start A. Cook, and were treated with TGFβ1. 1190 and 91 were identified lncRNAs in both sequencing datasets and filtered for 15 deregulated candidates. The dot plot illustrates all detected transcripts including the protein-coding. The dark grey dots represent the selected lncRNA candidates.

FIG. 3: Copy number PCR of LncFIB #4, #8, #10 and #11 performed in the following cell types: Human cardiac fibroblasts (HCFs) treated with either vehicle control or TGFβ1, induced pluripotent stem cell-derived cardiomyocytes, human vascular endothelial cells (HUVEC), Medical Research Council cell strain 5 (MRC-5) embryonic lung fibroblasts and human embryonic kidney cells (HEK293T). One-way ANOVA. N=3.

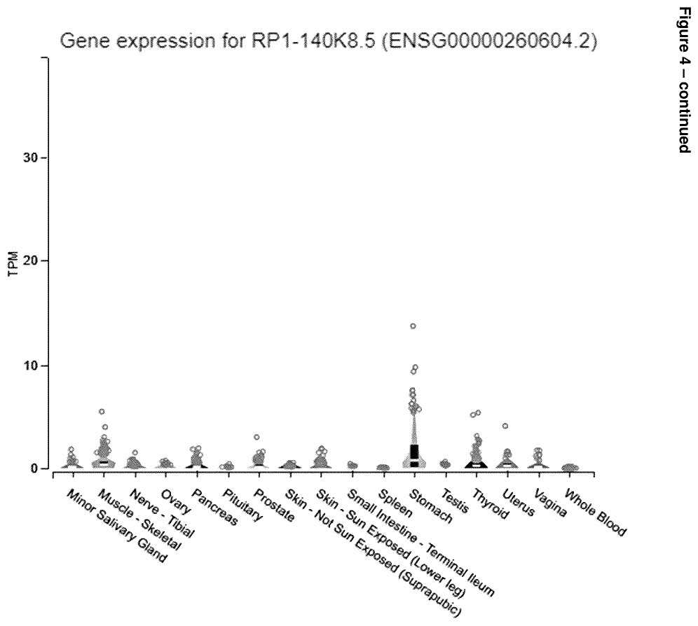

FIG. 4: GTex analysis of LncFIB #10 (SEQ ID NO: 2) expression patterns.

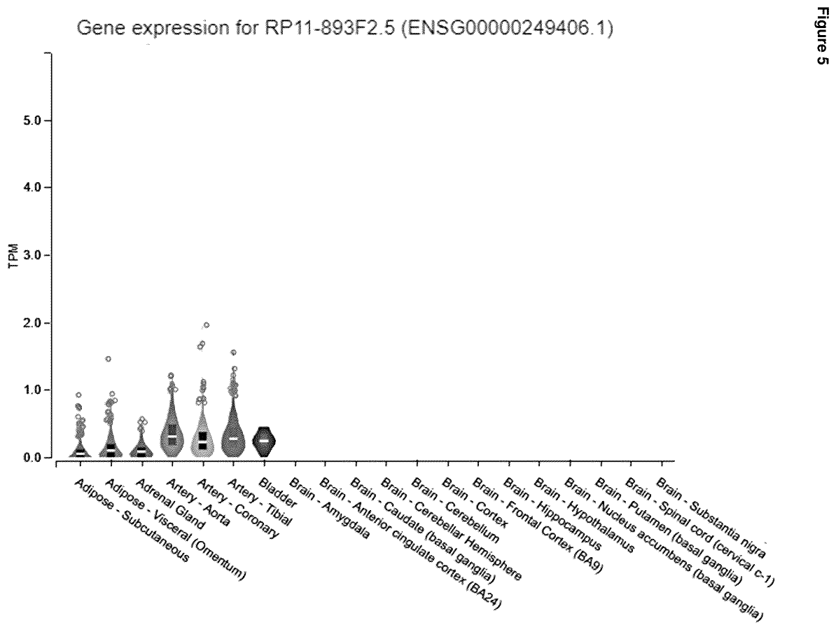

FIG. 5: GTex analysis of LncFIB #11 (SEQ ID NO: 1) expression patterns.

FIG. 6: LncFIB #4, #8, #10 and #11 induction in lung, dermal, liver and kidney fibroblasts upon high FBS or TGFβ1 treatment assessed via RT-qPCR. One-way ANOVA. N=3.

FIG. 7: Hypoxia-induced up- and downregulation of LncFIB #4, #8, #10 and #11 after 24 h of 0.2% hypoxia compared to normoxia. One-way ANOVA. N=3.

FIG. 8: Metabolic activity (WST) and cytotoxicity (LDH) of HCFs, MRC-5 and liver cells (HepG2) after treatment with GapmeR 10.1 and 11.1 compared to control A GapmeR. One-way ANOVA. N=3, for HCFs three independent LOTs.

FIG. 9: Treatment with GapmeRs #10.1 and #11.1 resulted in a strong knockdown of LncFIB #10 and #11 in HCFs. Common fibrosis marker genes associated with the activation of fibroblast, their proliferation, adhesion, migration and the matrix remodeling were significantly downregulated even when stimulated with TGFβ1. Two-way ANOVA. N=3, independent LOTs.

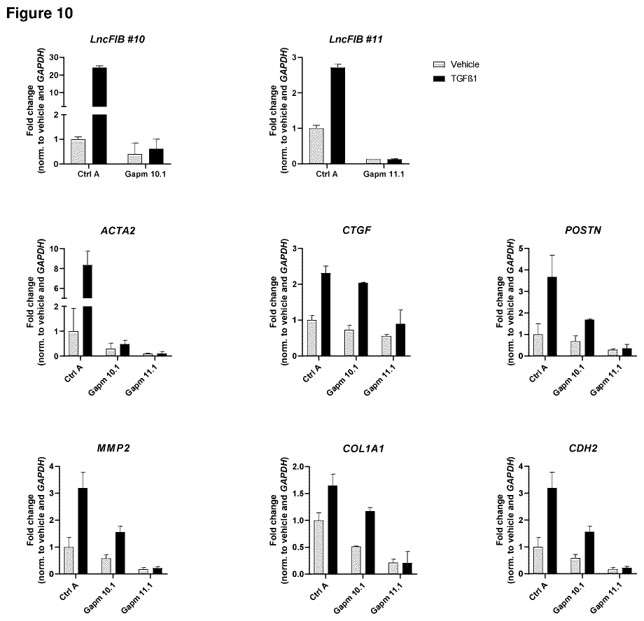

FIG. 10: Treatment with GapmeRs #10.1 and #11.1 resulted in a strong knockdown of LncFIB #10 and #11 in MRC5 lung fibroblasts. Common fibrosis marker genes associated with the activation of fibroblast, their proliferation, adhesion, migration and the matrix remodeling were significantly downregulated even when stimulated with TGFβ1.

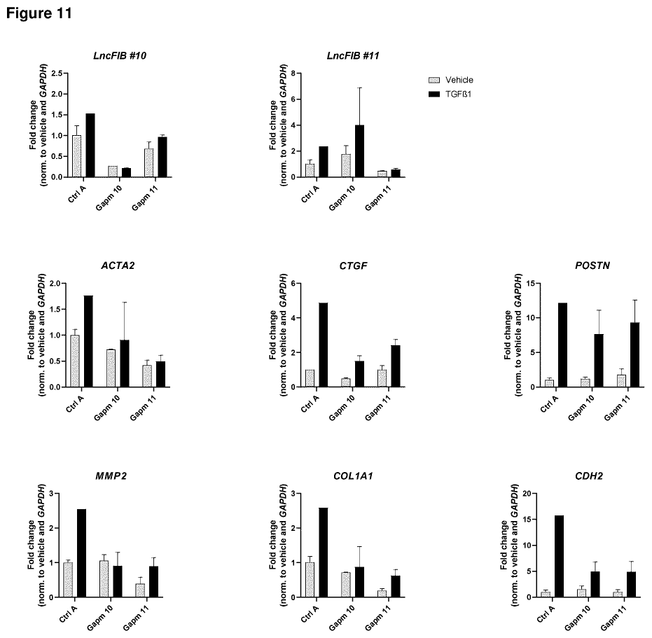

FIG. 11: Treatment with GapmeRs #10.1 and #11.1 resulted in a strong knockdown of LncFIB #10 and #11 in human primary liver fibroblasts (HPLF). Common fibrosis marker genes associated with the activation of fibroblast, their proliferation, adhesion, migration and the matrix remodeling were significantly downregulated even when stimulated with TGFβ1.

FIG. 12: Proliferation (BrdU) and migration assay of HCFs treated with GapmeRs #10.1 and #11.1 combined with a 48 h or 72 h TGFβ1 stimulation. BrdU incorporation was measured after 72 h of the combined treatment. The wound closure after a scratch was monitored for 20 h (started 48 h after treatment) with the Cytation 3, where an images was captured every 2 h. The analysis was performed with Fiji version 1.52p. Two-way ANOVA. N=3, independent LOTs (SG1, AS1 and AS2). AUC: area under the curve.

FIG. 13: Gel contraction assay of embedded HCFs in a collagen matrix revealed a strongly reduced contractile function of fibroblasts with or without additional TGFβ1 treatment upon prior lncFIB #10 and #11 inhibition.

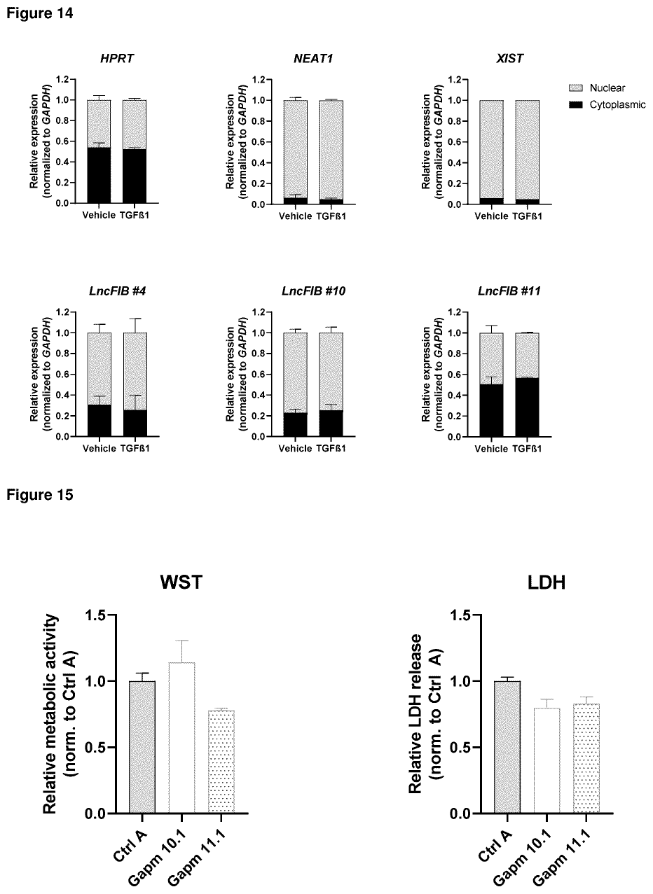

FIG. 14: Subcellular fractionation of HCFs treated with TGFβ1 for 72 h. RT-qPCR measured relative mRNA levels of the protein-coding gene HPRT, the nuclear localized lncRNAs NEAT1 and XIST (only in one female HCF LOT) and were compared to LncFIB #4, 10 and #11 distributions in the cytoplasm and the nucleus. N=3, independent LOTs.

FIG. 15: Effect of the GapmeR treatment (GapmeRs #10.1 and #11.1) on the metabolic activity (WST) and cytotoxicity (LDH) in cancer-associated fibroblatsts. n=1.

FIG. 16: Treatment with GapmeRs #10.1 and #11.1 resulted in a strong knockdown of LncFIB #10 and #11 in CAFs. Common fibrosis marker genes associated with the activation of fibroblast, their proliferation, adhesion, migration and the matrix remodelling were downregulated even when stimulated with TGFβ1. n=1.

The examples illustrate the invention.

### Example 1—Experimental Background and Design

Functionally, the potential to proliferate and migrate, as well as the secretion of extracellular matrix components and respective modifiers can be used to distinguish distinct stages of fibroblast activation. In combination with specific marker gene expressions, this led to a definition of four stages of fibroblast activation, which is applicable to other organ systems such as the lung, kidney and liver as well: 1) Initiation, 2) Inflammation, 3) Proliferation and 4) Modification (FIG. 1).⁸

The initiation is characterized by an induction of an early gene program, which directly induces inflammatory processes as a second step. Several cell types release cytokines within the injured tissue, attracting immune cells on the one hand and on the other hand, inducing the migration of fibroblasts towards the site of injury, where the proliferation of the cells contributes to a wound closure. The migration and proliferation are typical features of a maintained activation state of fibroblasts, then referred to as myofibroblasts. These cells can be distinguished from quiescent fibroblasts by their expression of contractile fibers crucial for wound closure, which consist of smooth muscle alpha actin (α-SMA).^{8,12} Chronic exposure to stressors and thereby the continuous release of pro-fibrotic signaling molecules by surrounding cell types leads to a permanent change of the fibroblast phenotype. A new differentiation state has been described in recent studies, where the authors defined those persistent cells as matrifibrocytes exhibiting less proliferative characteristics. Instead, they remain as resistant cells within the myocardium forming massive amounts of extracellular matrix.⁸ At this point, it is very difficult to reverse the severe remodeling processes, explaining the major challenges to develop effective drugs against cardiac fibrosis and concomitant heart failure.

Despite cardiac injury and chronic tissue stretching, several growth factors induce the activation of fibroblasts. The most important driver is the transforming growth factor beta 1 (TGFβ1), which is released by nearly all cell types within the heart, including infiltrating macrophages.¹⁵ Notably, TGFβ1 is secreted in a latent form and is stored within the extracellular matrix bound to distinct binding proteins, where it is awaiting its processing. This rapid activation and availability allows an immediate response to stress stimuli.¹⁶ Although this system is quite sophisticated, TGFβ1 is a pleiotropic regulator of growth, differentiation and apoptosis, and thus, not the first choice drug target.¹⁷ Despite its obvious limitations, this powerful pathway can still be the focus of therapeutic interventions by targeting downstream effectors with preferably fibroblast-specific actions. Here, (long) non-coding RNAs seem to be a very promising tool to interfere with the fibrotic responses because of their mainly restricted range of function and a high specificity in developmental and pathological contexts.¹⁸

The expansion of the non-coding RNA field in the past years was enabled by a continuous technical improvement of the genomic and transcriptomic profiling and the public availability in huge databases. Long non-coding RNAs are indeed the largest and most diverse class of ncRNAs, but not the best investigated one. MicroRNAs (miRNAs) are short single-stranded RNAs with a size of 21-23 nucleotides. The canonical mode of action is to bind mRNAs and facilitate their degradation or inhibition.^{19,20} Recently, additional functions have been discovered including epigenetic or transcriptional modulation within the nucleus.²¹ Clinical trials comprise diverse implications for miRNA-based therapies e.g. antisense oligonucleotides (ASOs) against miR-122 for the treatment of Hepatitis C virus (HCV) infection (phase II)^{22,23} or anti-miR-17 for the treatment of polycystic kidney disease (phase I).^{24,25} These studies further underpin the clinical relevance of ASOs for targeting specific ncRNAs. In addition to miRNAs, lncRNAs are less characterized so far, but their exceptional potential has already been highlighted in many studies. The classification of this group is only based on size, including all non-coding transcripts larger than 200 nucleotides. Therefore, it is not surprising that multifaceted functions can be found in this class. LncRNAs are able to interact with each type of molecule, from RNA to DNA and finally small peptides and proteins, or complexes of such either in the nucleus, the cytoplasm or in other organelles.¹⁸

The group of Stuart A. Cook collected fibroblast from a very large patient cohort undergoing surgical intervention.²⁶ The primary human cardiac fibroblasts (HCFs) have been cultured and treated with TGFβ1 or a respective control solution. A subsequent RNA-sequencing analysis revealed highly deregulated protein-coding transcripts, and led to the discovery of IL-11 that is directly induced by TGFβ1 and orchestrates its pro-fibrotic effects.^{15,26} Since IL-11 functions are spread across organs, the specificity of a therapy and associated side effects are relatively uncertain. Furthermore, protein inhibitors such as antibodies bring along well-known problems of immunological reactions and difficulties in production.^{27,28} In contrast, targeting RNA molecules persuades with the advantage of a simple inhibitor design and production, and less safety concerns.^{29,30}

Identification of fibrosis-associated lncRNAs herein was based on datamining and a re-analysis of the publicly available RNA-seq data set provided by Cook et al.,²⁶ since most lncRNAs can be detected in in-depth sequencing approaches. 15 strongly up- and downregulated candidates were identified (SEQ ID NOs 1 to 17). Validating primers and expression levels of annotated lncRNAs as well as functional in vitro assays were then used for selected lncRNAs of the 15 lncRNAs. In particular, two lead candidates, LncFIB #10 (SEQ ID NO: 1) and #11 (SEQ ID NO: 1), have been selected for further characterization with specific inhibitors based on ASOs named GapmeRs.

### Example 2—Results and Discussion

### Example 2.1—Identification of 15 Highly Deregulated lncRNA Candidates in a RNA-Seq Reanalysis of Cardiac Fibroblasts Treated with TGFβ1

The group of Stuart A. Cook performed a RNA-seq analysis, where they identified TGFβ1-responsive protein-coding transcripts. However, our reanalysis of the two datasets allowed us to select 15 highly deregulated lncRNAs illustrated in Table 1. The reanalysis identified three downregulated (LncFIB #1-3) and twelve upregulated lncRNAs (LncFIB #4-15). Most of the candidates are novel transcripts meaning that they are not associated with any functions yet. The following result section mainly concentrates on LncFIB #10 and #11, but some analyses are additionally presented for LncFIB #4 and #8 as a comparison.

TABLE 1
Summary of the 15 selected IncRNA candidates, named LncFIB #1-15. The IncRNAs were sorted according to
their ratio between untreated and TGFβ1-stimulated samples, and their total counts during RNA-seq analysis.

| | | | 2 patients | | 84 patients | | | |
|---|---|---|---|---|---|---|---|---|
| LncFIB # | Gene ID | Symbol | Ratio | Counts | Ratio | Counts | Type (ncRNA) | Description |
| 1 | ENSG00000227811 | INKA2-AS1 | 0.27 | 173.25 | 0.53 | 52.62 | bidirectional_pro- | INKA2 antisense RNA 1 |
| | | | | | | | moter_IncRNA | |
| 2 | ENSG00000253819 | LINC01151 | 0.14 | 150.00 | 0.39 | 62.07 | lincRNA | long intergenic non-protein |
| | | | | | | | | coding RNA 1151 |
| 3 | ENSG00000250056 | LINC01018 | 0.23 | 663.00 | 0.54 | 826.88 | lincRNA | long intergenic non-protein |
| | | | | | | | | coding RNA 1018 |
| 4 | ENSG00000251281 | AC034223.2 | 4.70 | 31.75 | 3.56 | 45.35 | lincRNA | novel transcript |
| 5 | ENSG00000268941 | LINC01711 | 3.18 | 44.50 | 2.90 | 67.93 | lincRNA | long intergenic non-protein |
| | | | | | | | | coding RNA 1711 |
| 6 | ENSG00000262061 | AC129507.1 | 7.50 | 22.50 | 5.18 | 54.80 | processed_transcript | uncharacterized LOC100506388 |
| 7 | ENSG00000250697 | AC010343.3 | 4.21 | 50.50 | 3.45 | 77.79 | lincRNA | novel transcript |
| 8 | ENSG00000259518 | LINC01583 | 1.97 | 148.00 | 1.83 | 69.89 | lincRNA | long intergenic non-protein |
| | | | | | | | | coding RNA 1583 |
| 9 | ENSG00000234840 | LINC01239 | 2.68 | 142.00 | 3.06 | 169.60 | lincRNA | long intergenic non-protein |
| | | | | | | | | coding RNA 1239 |
| 10 | ENSG00000260604 | AL590004.3 | 1.57 | 320.75 | 2.18 | 832.20 | lincRNA | novel transcript |
| 11 | ENSG00000249406 | AC015909.1 | | | 1.69 | 101.36 | lincRNA | novel transcript |
| 12 | ENSG00000233901 | LINC01503 | | | 1.68 | 175.36 | lincRNA | long intergenic non-protein |
| | | | | | | | | coding RNA 1503 |
| 13 | ENSG00000248187 | AC078850.1 | | | 1.93 | 205.56 | lincRNA | novel transcript |
| 14 | ENSG00000251615 | AC104825.1 | | | 2.00 | 318.85 | lincRNA | novel transcript |
| 15 | ENSG00000249669 | CARMN | | | 2.16 | 644.31 | lincRNA | cardiac mesoderm enhancer- |
| | | | | | | | | associated nc RNA |

### Example 2.2—Enrichment of LncFIB #10 and #11 in Activated Cardiac Fibroblasts by Treatment with TGFβ1 In Vitro

The before mentioned datamining approach revealed the differential expression of 15 lncRNA candidates in response to TGFβ1 stimulation in human cardiac fibroblasts (HCFs), whereof three lncRNAs were downregulated and twelve were found to be upregulated. For the validation, several primer pairs have been designed and tested via quantitative real-time PCR (RT-qPCR). According to the respective melting curves, sequencing results of the transcripts and the expression levels in HCFs (data not shown here), four lncRNAs have been chosen for further functional characterization.

First, the induction of lncRNA expression by TGFβ1 was considered as the most important criteria to confirm the RNA-seq results and the potential roles in cardiac fibrosis. The basal expression of LncFIB #10 is relatively low; however, stimulation with TGFβ1 caused more than a 20-fold upregulation of the gene expression. In contrast, LncFIB #11 seemed to have higher levels in unstimulated HCFs, but the induction was less pronounced. Second, expression levels of the respective candidates were compared to other human cell types including induced pluripotent stem cell (iPSC)-derived cardiomyocytes, endothelial cells (HUVECs), lung fibroblasts (MRC-5) and kidney cells (HEK293T). For better comparison between different cell types, the RT-qPCR results were not normalized to a certain housekeeping gene. Instead, lncRNA-specific copy number plasmids have been generated, allowing a direct determination of the exact copy numbers per μg RNA, thus circumventing the well-known problem of differentially regulated housekeeper expressions. Both candidates were enriched in cardiac fibroblasts especially when these became activated. Noteworthy, LncFIB #11 seemed to be the most specific candidate in cardiac fibroblasts since LncFIB #10 displayed even higher expression levels in HUVECs. Therefore, a possible function in endothelial cells has been evaluated in a following experiment. The two lncRNAs LncFIB #4 and #8 showed a strong induction upon TGFβ1 stimulation and presented a relatively specific expression in activated fibroblasts. Nevertheless, these two candidates were excluded from follow-up experiments due to the ineffectiveness of the GapmeRs.

### Example 2.3—GTex Online Tool Revealed a lncRNA Expression Pattern Relatively Restricted to Cardiac Tissue and Cultured Fibroblasts

The Genotyp-tissue Expression (GTex) project provides information about expression patterns of several transcripts in 54 selected non-diseased tissue sites. This comprehensive and still ongoing project is publicly available and combines several analyses tools and databases. Here, both lncRNAs presented a quite selective expression within the heart and cultured fibroblasts. While LncFIB #11 was also abundant in several other tissues, LncFIB #10 was more restricted to the heart.

### Example 2.4—In Vitro Induction of lncRNA Candidates with High FBS and TGFβ1 in Different Types of Human Fibroblasts

The nature of fibroblasts in different organs is relatively similar, assuming that the lncRNA candidates might also play a role in these cells. Therefore, certain types of human primary fibroblasts originating from lung, skin, liver and kidney were stimulated either with a high serum concentration (high FBS) or with TGFβ1. Lung fibroblasts represent an important driver of lung fibrosis in different diseases. Therefore, an upregulation of the lncRNAs in response to TGFβ1 was expected. Indeed, all four candidates were significantly induced upon stimulation. In contrast, only LncFIB #4 and #8 expression was elevated in dermal fibroblasts, but the liver and kidney fibroblasts even responded less to the stimuli. In summary, the TGFβ1-induction of lncRNA candidates seemed to be variable between different types of fibroblasts, but the general fold change was remarkably higher in cardiac fibroblasts. Here, distinct pre-activation states of the fibroblasts have to be considered as a possible factor influencing the responsiveness to the stimuli, and additionally, overall differences in the reaction to this in vitro activation as well.

### Example 2.5—Hypoxia-Stimulated Upregulated of LncFIB #10 and #11 in HUVECs

Endothelial dysfunction is part of the pathogenesis within the heart contributing to its remodeling processes including fibrosis. Therefore, it is not unlikely that lncRNAs might exhibit additional functions in these cells. An established in vitro hypoxia model was used to evaluate a potential complementary role in endothelial cells, in this case in HUVECs. Hypoxia resembles the lack of nourishment during and after a cardiac event. An early time point (24 h) has been chosen for identifying direct responses to this stimulus. The qPCR results showed a 2-fold upregulation of LncFIB #10, whereas the LncFIB #11 expression levels were below the precise detection range. Considering the remarkable induction of LncFIB #10 expression in cardiac fibroblast compared to the hypoxia-dependent upregulation in endothelial cells, the latter direction was not pursued in following studies.

### Example 2.6—Effect of LncFIB #10 and #11-Specific GapmeRs on Metabolic Activity and Cell Viability in Cardiac and Lung Fibroblasts Compared to Liver Cells

Assessing the metabolic activity of fibroblasts is useful to evaluate the impact of lncRNA inhibition on fibrosis. In primary human cardiac fibroblasts, GapmeR #10.1 and #11.1 treatments were associated with a diminished metabolic activity. A similar, but more pronounced effect was observed in a human embryonic lung fibroblast cell line (MRC5) with significant changes in WST-1 metabolization. Excluding a cytotoxic effect, a CellTox green or LDH release assay were additionally performed, both measuring the amount of dead cells in a quantitative or qualitative manner, respectively. Both cell types showed an increased number of dying cells when treated with GapmeR 11.1, but not with GapmeR 10.1. Whether these cytotoxic properties are restricted to only fibroblasts considering it as a powerful anti-fibrotic treatment or are caused by the GapmeR 11.1 itself, was part of the next investigations.

Continuing with the functional characterization in vitro, the GapmeRs were tested in a human liver cancer cell line (HepG2). Firstly, hepatotoxic effects appear quite frequently when applying LNAs or GapmeRs in vivo, and secondly, this experiment helped to confirm or disprove a fibroblasts-restricted function of the lncRNA candidates. The metabolic activity was slightly impaired upon GapmeR #11.1 treatment. In addition, an enhanced LDH release indicated an increased cellular death rate assuming that the compromised activity of fibroblasts and hepatic cells is at least partially a side effect of the GapmeR cytotoxicity. Since GapmeR 10.1 had no impact on HepG2 metabolic activity and appeared to be even less toxic than the control A GapmeR, LncFIB #10 is supposed to act fibroblast

### Example 2.7—Therapeutic LncFIB #10 and #11 Inhibition is Effective and Reduces Fibrosis Marker Gene Expression In Vitro

After in vitro evaluation of GapmeR cytotoxicity, functional experiments have been conducted in HCFs originating from three different human donors. Initially, the expression of the most important fibrosis marker genes was examined. Since lncRNA knockdown is supposed to have an anti-fibrotic effect, the HCFs were additionally activated with TGFβ1. The treatment with lncRNA GapmeRs and 5 ng/mL TGFβ1 was carried on for 72 h in total. The widely used marker for myofibroblasts, ACTB, which was already introduced before, was three-times upregulated in activated fibroblasts. However, GapmeR 10.1 and 11.1 significantly decreased the expression also in combination with TGFβ1 stimulation, resulting in even lower levels than in the basal condition (vehicle and control A GapmeR). Similar results were observed for the TGFβ1-induced connective tissue growth factor (CTGF), further indicating a role of LncFIB #10 and #11 in TGFβ1-mediated signaling cascades. Furthermore, factors being involved in adhesion and migration (POSTN and CDH2) as well as in matrix remodeling (MMP2 and COLA1) showed a strongly reduced induction by TGFβ1 when previously treated with the GapmeRs 10.1 and 11.1. Similar results could be obtained in MRC5 lung fibroblasts and human primary liver fibroblasts, further supporting the functional relevance of the two lncRNAs in fibrotic conditions.

### Example 2.8—Functional In Vitro Analysis Revealed a Reduced Proliferation, Migration and Contractile Function after LncFIB #10 and #11 Knockdown

The impact of LncFIB #10 and #11 inhibition on fibrosis markers is indeed a strong indicator for an anti-fibrotic effect, but activated fibroblasts exhibit additional features that can be used to prove the contribution to this phenotype. Fibrosis is characterized by an excessive proliferation of fibroblasts, which was investigated by an in vitro BrdU incorporation assay. In this experiment, only GapmeR 11.1 had a significant anti-proliferative effect. Considering the observed cytotoxic effect, it is unclear whether LncFIB #11 indeed has an impact on proliferation or whether GapmeR 11.1 causes too much cellular stress for the entry into the cell cycle. Moreover, limitations of the BrdU assay are the short period of observation, which is only 24 h after labeling the cells, although the population doubling time of HCFs is around 48 h, and the cytotoxicity of the BrdU labeling solution itself. Alternative proliferation assays might be considered at this point.

Another important feature of fibroblast activation is their migratory ability. This was assessed in a scratch assay, where the wound closure was monitored for 20 h. Prior to this assay, the GapmeRs have been applied in combination with a TGFβ1 stimulation, which should further promote HCF migration. Although, TGFβ1 did not induce the expected effect, a significantly prolonged wound closure was detected for both GapmeR treatments independent of TGFβ1.

Directly after an activation of fibroblasts and their differentiation into myofibroblasts, these cells start to express αSMA, a contractile fiber that is not only eponymous for this state of differentiation but also responsible for cellular contractions allowing wound closure. This ability can be reduced by anti-fibrotic drugs, which was assessed in an in vitro gel contraction assay. For this purpose, HCFs were transfected with the GapmeRs and after 72 h, these cells were embedded within a collagen gel. The medium was supplemented with TGF or vehicle control. Next, the gel was carefully detached from the plastic following a 48 h treatment period. The monitoring was performed with an automated live cell imager (Cytation) in total for 24 h. The activated state of fibroblasts was majorly reduced upon GapmeR-mediated lncFIB #10 and #11 knockdown compared to control cells, which is indicated by a fewer size reduction of the gel. This result confirms the diminished expression of fibrosis marker genes quantified via qPCR and therefore highlights the great therapeutic potential of lncFIB #10 and #11 inhibitors.

### Example 2.9—Distribution of lncRNAs in the Cytoplasm and Nucleus of Cardiac Fibroblasts

The subcellular localization provided first insights into possible general functions of the respective lncRNAs. As controls, the protein-coding housekeeper HPRT, and two nuclear-enriched lncRNAs, NEAT1 and XIST, were chosen and the RT-qPCR analysis revealed expected distributions of the controls. The depicted candidates presented variable allocations with LncFIB #4 and #10 being more nuclear-enriched, whereas LncFIB #11 showed an even distribution. A nuclear localization points towards an epigenetic or transcriptional regulator in contrast to a cytoplasmic lncRNA probably affecting the translation of or the interaction with proteins in diverse contexts.

### Example 2.10—Inhibition of TGFβ1-Induced Long Non-Coding RNAs Prevents Cardiac Fibrosis

Treatment of cancer-associated fibroblasts (CAF, from lung adenocarcinoma) with the investigated anti-fibrotic GapmeRs, which delivered promising results in human cardiac fibroblasts, embryonal lung fibroblasts and primary liver fibroblasts, was not toxic since LDH release was even decreased (FIG. 1). Furthermore, the CAFs treated with GapmeR 11 showed a reduced metabolic activity (WST assay, FIG. 15). The successful knockdown of the respective lncRNA candidate was confirmed via qPCR. Here, a downregulation of common fibrosis marker genes was additionally observed (especially ACTA2, CTGF and COL1A1). Most importantly, pro-fibrotic TGFβ1 treatment induced the expression of both lncFIB #10 and #11 (FIG. 16). This induction was rather low compared to cardiac and lung fibroblasts, but confirms the finding that lncFIB #10 and #11 are TGFβ1-inducible.

### Example 3—Conclusion

The datamining approach as described identified 15 differentially expressed long non-coding RNAs in human cardiac fibroblasts when activated with TGFβ1. Their relatively specific expression in fibroblasts was confirmed with the highest in vitro induction in cardiac cells and a surprisingly high induction upon TGFβ1 stimulation. Specific lncRNA inhibitors, modified LNAs named GapmeRs, were used for functional analyses. Here, two lead candidates have been discovered as fibrosis-associated transcripts, referred to as LncFIB #10 (SEQ ID NO: 2) and #11 (SEQ ID NO: 1). Knockdown of both lncRNAs concurrently reduced fibrosis marker expression in either cardiac or lung fibroblast. Furthermore, their inhibition led to an impaired metabolic activity, migration and proliferation of cardiac fibroblasts. In this context, no cytotoxic effects occurred for GapmeR 10.1, but GapmeR 11.1 seemed to have slightly (hepato-) toxic features. In this case, alternative knockdown strategies might be pursued to figure out, whether LncFIB #11 is such a crucial player in regulating fibrotic gene programs that the viability is simultaneously affected, or whether the inhibition itself is toxic.

### REFERENCES

1. Quek, J. et al. The Association of Plant-Based Diet With Cardiovascular Disease and Mortality: A Meta-Analysis and Systematic Review of Prospect Cohort Studies. Frontiers in Cardiovascular Medicine 8, (2021).
2. Mattioli, A. v., Sciomer, S., Cocchi, C., Maffei, S. & Gallina, S. Quarantine during COVID-19 outbreak: Changes in diet and physical activity increase the risk of cardiovascular disease. Nutrition, Metabolism and Cardiovascular Diseases 30, 1409-1417 (2020).
3. WHO. Cardiovascular diseases. (2021).
4. Breckwoldt, K., Weinberger, F. & Eschenhagen, T. Heart regeneration. Biochimica et Biophysica Acta (BBA)—Molecular Cell Research 1863,1749-1759 (2016).
5. Bhatt, A. S., Ambrosy, A. P. & Velazquez, E. J. Adverse Remodeling and Reverse Remodeling After Myocardial Infarction. Current Cardiology Reports 19, 71 (2017).
6. Fuchs, F. D. & Whelton, P. K. High Blood Pressure and Cardiovascular Disease. Hypertension 75, 285-292 (2020).
7. Nakamura, M. & Sadoshima, J. Mechanisms of physiological and pathological cardiac hypertrophy. Nature Reviews Cardiology 15, 387-407 (2018).
8. Fu, X. et al. Specialized fibroblast differentiated states underlie scar formation in the infarcted mouse heart. Journal of Clinical Investigation 128, (2018).
9. Niederberger, P. et al. Heart Transplantation With Donation After Circulatory Death. Circulation: Heart Failure 12, (2019).
10. Zuchi, C. et al. Role of endothelial dysfunction in heart failure. Heart Failure Reviews 25, 21-30 (2020).
11. Clinical Study to Assess Safety, PK and PD Parameters of CDR132L. (2020).
12. Travers, J. G., Kamal, F. A., Robbins, J., Yutzey, K. E. & Blaxall, B. C. Cardiac Fibrosis. Circulation Research 118, 1021-1040 (2016).
13. Webber, M., Jackson, S. P., Moon, J. C. & Captur, G. Myocardial Fibrosis in Heart Failure: Anti-Fibrotic Therapies and the Role of Cardiovascular Magnetic Resonance in Drug Trials. Cardiology and Therapy 9, 363-376 (2020).
14. Parichatikanond, W., Luangmonkong, T., Mangmool, S. & Kurose, H. Therapeutic Targets for the Treatment of Cardiac Fibrosis and Cancer: Focusing on TGF-β Signaling. Frontiers in Cardiovascular Medicine 7, (2020).
15. Dobaczewski, M., Chen, W. & Frangogiannis, N. G. Transforming growth factor (TGF)-β signaling in cardiac remodeling. Journal of Molecular and Cellular Cardiology 51, (2011).
16. Annes, J. P., Munger, J. S. & Rifkin, D. B. Making sense of latent TGFβ activation. Journal of Cell Science 116, (2003).
17. Frangogiannis, N. G. Targeting the transforming growth factor (TGF)-β cascade in the remodeling heart: Benefits and perils. Journal of Molecular and Cellular Cardiology 76, (2014).
18. Bär, C., Chatterjee, S. & Thum, T. Long Noncoding RNAs in Cardiovascular Pathology, Diagnosis, and Therapy. Circulation 134, (2016).
19. Zhou, S. et al. miRNAS in cardiovascular diseases: potential biomarkers, therapeutic targets and challenges. Acta Pharmacologica Sinica 39, (2018).
20. Melman, Y. F., Shah, R. & Das, S. MicroRNAs in Heart Failure. Circulation: Heart Failure 7, 203-214 (2014).
21. Khan, A. W. Nuclear functions of microRNAs relevant to the cardiovascular system. Translational Research 230, (2021).
22. Hoffmann-La Roche. https://clinicaltrials.gov/ct2/show/NCT02508090. (2017).
23. van der Ree, M. H. et al. Miravirsen dosing in chronic hepatitis C patients results in decreased microRNA-122 levels without affecting other microRNAs in plasma. Alimentary Pharmacology & Therapeutics 43, (2016).
24. Lee, E. C. et al. Discovery and preclinical evaluation of anti-miR-17 oligonucleotide RGLS4326 for the treatment of polycystic kidney disease. Nature Communications 10, (2019).
25. Regulus Therapeutics Inc. https://clinicaltrials.gov/ct2/show/NCT04536688. (2020).
26. Schafer, S. et al. IL-11 is a crucial determinant of cardiovascular fibrosis. Nature 552, (2017).
27. Kalden, J. R. & Schulze-Koops, H. Immunogenicity and loss of response to TNF inhibitors: implications for rheumatoid arthritis treatment. Nature Reviews Rheumatology 13, 707-718 (2017).
28. Boehncke, W.-H. & Brembilla, N. C. Immunogenicity of biologic therapies: causes and consequences. Expert Review of Clinical Immunology 14, 513-523 (2018).
29. Bennett, C. F. Therapeutic Antisense Oligonucleotides Are Coming of Age. Annual Review of Medicine 70, 307-321 (2019).
30. Quemener, A. M. et al. The powerful world of antisense oligonucleotides: From bench to bedside. WIREs RNA 11, (2020).

1. A pharmaceutical composition comprising
(i) a compound inhibiting the expression and/or the activity of one or more lncRNAs selected from SEQ ID NOs 1 to 14; and/or
(ii) a compound promoting the expression and/or the activity of one or more long non-coding RNAs (lncRNAs) selected from SEQ ID NOs 15 to 17.

2. A method of treating or preventing a fibrotic disorder, a tumor, or cancer comprising administering to a subject
(i) a compound inhibiting the expression and/or the activity of one or more lncRNAs selected from SEQ ID NOs 1 to 14; and/or
(ii) a compound promoting the expression and/or the activity of one or more long non-coding RNAs (lncRNAs) selected from SEQ ID NOs 15 to 17.

3. The method of claim 2, wherein the fibrotic disorder is selected from systemic sclerosis (SSc), sclerodermatous graft vs. host disease, and nephrogenic systemic fibrosis, as well as numerous organ-specific disorders including radiation-induced fibrosis, and organ fibrosis wherein the organ fibrosis is preferably the cardiac fibrosis, pulmonary fibrosis, liver fibrosis, kidney fibrosis, gastrointestinal fibrosis, skeletal muscle fibrosis and multifocal fibrosclerosis.

4. The method of claim 2, wherein the cancer is a metastatic cancer or a fibrotic cancer, preferably myelofibrosis.

5. The pharmaceutical composition of claim 1, wherein the compound as defined in (i) is a small molecule inhibitor, a nucleotide-based inhibitor or an amino acid-based inhibitor.

6. The pharmaceutical composition of claim 5, wherein the nucleotide-based inhibitor is an aptamer, a siRNA, a shRNA, a miRNA, a ribozyme, an antisense nucleic acid molecule, a CRISPR-Cas-based construct, a meganuclease, a zinc finger nuclease, or a transcription activator-like (TAL) effector (TALE) nuclease, and/or the amino acid-based inhibitor is an aptamer, an antibody or an antibody mimetic.

7. The pharmaceutical composition of claim 5, wherein the nucleotide-based inhibitor comprises
(a) a nucleic acid sequence which comprises or consists of a nucleic acid sequence being complementary to at least 12 continuous nucleotides of a nucleic acid sequence selected from SEQ ID NOs 1 to 14,
(b) a nucleic acid sequence which comprises or consists of a nucleic acid sequence which is at least 70% identical to the complementary strand of one or more nucleic acid sequences selected from SEQ ID NOs 1 to 14,
(c) a nucleic acid sequence which comprises or consists of a nucleic acid sequence according to (a) or (b), wherein the nucleic acid sequence is DNA or RNA,
(d) an expression vector expressing the nucleic acid sequence as defined in any one of (a) to (c), or
(e) a host comprising the expression vector of (d).

8. The pharmaceutical composition of claim 1, wherein the compound as defined in (ii) is
(a) a nucleic acid sequence which comprises or consists of a nucleic acid sequence that is at least 70% identical to one or more lncRNAs selected from SEQ ID NOs 15 to 17,
(b) an expression vector expressing the nucleic acid sequence as defined in (a), or
(c) a host comprising the expression vector of (b).

9. The pharmaceutical composition of claim 1, wherein the compound as defined in (ii) is
(a) a transcription factor promoting the expression of one or more lncRNAs selected from SEQ ID NOs 15 to 17, and/or
(b) a small molecule enhancing the expression of one or more lncRNAs selected from SEQ ID NOs 15 to 17.

10. A method for diagnosing a fibrotic disorder, a tumor, or cancer in a patient, comprising
(a) detecting the expression level of one or more lncRNAs selected from SEQ ID NOs 1 to 17 in a sample obtained from said patient, and
(b) comparing said expression level of the one or more lncRNAs with the expression level of these one or more lncRNAs in a sample obtained from healthy subjects or predetermined standard, wherein
a greater than 2-fold upregulation of one or more lncRNAs selected from SEQ ID NOs 1 to 14; and/or
a greater than 2-fold downregulation of one or more lncRNAs selected from SEQ ID NOs 15 to 17 is indicative for a fibrotic disorder, a tumor, or cancer in the patient.

11. The method of claim 10, wherein the detection of the expression level of the one or more lncRNAs comprises
(a) quantitative PCR, preferably quantitative real time PCR, or
(b) a template/RNA amplification method followed by determining the expression level of the one or more lncRNAs using a fluorescence- or luminescence-based quantification method.

12. The method of claim 10, wherein the method comprises prior to the detection of the expression level of the long non-coding RNA a pre-amplification step of the RNA within the test patient's sample and/or the control patient's sample.

13. (canceled)

14. (canceled)

15. The pharmaceutical composition of claim 1, wherein the one or more lncRNAs are at least 2 lncRNAs.

16. The pharmaceutical composition of claim 15, wherein the one or more lncRNAs are at least 5 lncRNAs.

17. The pharmaceutical composition of claim 6, wherein the antibody mimetic is selected from affibodies, adnectins, anticalins, DARPins, avimers, nanofitins, affilins, Kunitz domain peptides, Fynomers®, trispecific binding molecules and probodies.

18. The pharmaceutical composition of claim 7, wherein the expression vector expressing the nucleic acid sequence in (d) is under the control of a heart-specific promoter and/or a fibroblast and/or fibrocyte-specific promoter.

19. The pharmaceutical composition of claim 8, wherein the compound as defined in (ii) is a nucleic acid sequence which comprises or consists of a nucleic acid sequence that is at least 80% identical to one or more lncRNAs selected from SEQ ID NOs 15 to 17.

20. The pharmaceutical composition of claim 8, wherein the compound as defined in (ii) is a nucleic acid sequence which comprises or consists of a nucleic acid sequence that is at least 90% identical to one or more lncRNAs selected from SEQ ID NOs 15 to 17.

21. The pharmaceutical composition of claim 8, wherein the compound as defined in (ii) is a nucleic acid sequence which comprises or consists of the nucleic acid sequence of one or more lncRNAs selected from SEQ ID NOs 15 to 17.

22. The pharmaceutical composition of claim 8, wherein the expression vector expressing the nucleic acid sequence in (b) is under the control of a heart-specific promoter and/or a fibroblast and/or fibrocyte-specific promoter.
